SEQUENCE LISTING (5 sequences)

SEQ ID NO: 1 - DNA, 12962 bp, Homo sapiens
gtcgacttaaccctagaaagataatcatattgtgacgtacgttaaagataatcatgcgtaaaattgacgcatgtgttttatcgatctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcggtaccgcatgcatcgatgctagcgcgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggaggatccggagccacgaacttctctctgttaaagcaagcaggagacgtggaagaaaaccccggtcctatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattccggactcggcaagcctatccctaaccctctgctgggcctggacagcaccgatccaaaaaagaagagaaaggtagaccctaagaagaagaggaaggtggaccccaagaagaagagaaaggtgtgaggcagtggagggagtggagcaaccaatttttcactcctgaagcaagctggggatgtagaggagaatcctgggcctatggactacaaagacgatgacgacaagcttggcactagtggctttgcgaatgaattgggacctaggttgatgggcaagctttctagtcaactcgagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagcggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgggtaacgccagggttttcccagtcacgacgttgtaaaacgacggccagtgccaagcttgcatgcctgcagtaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaggtaccaccggtcctgcaggttagacgttgatcctggcgctggcgcaagcagcagggtgtctatccatgccgctctcctgggcgcagctcatgggcagggtgccggcggcctgctcctccacctcgggagggaagccgtgagaattcacggcgatcttgccgcccttcttggccttaatgagaatctcgcggatcttgcgggcgtccaacttgccggtcagtcctttaggcacctcgtccacgaacacaacaccaccgcgcagcttcttggcggttgtaacctggctggccacatagtccacgatctccttctcggtcatggttttaccgtgttccagcacgacgactgcggcgggcagctcgccggcatcgtcgtcgggcaggccggcgaccccggcgtcgaagatgttggggtgttgcagcaggatgctctccagttcggctggggctacctggtagcccttgtatttgatcaggctcttcagccggtccacgatgaagaagtgctcgtcctcgtcccagtaggcgatgtcgccgctgtgcagccagccgtccttgtcgatgagagcgtttgtagcctcggggttgttaacgtagccgctcatgatcatggggccacggacgcacagctcgccgcgctggttcacacccagtgtcttaccggtgtccaagtccaccaccttagcctcgaagaagggcaccaccttgcctactgcgccaggcttgtcgtccccttcgggggtgatcagaatggcgctggttgtttctgtcaggccgtagccctggcggatgcctggtaggtggaagcgtttggccacggcctcacctacctccttgctgagcggcgccccgccgctggcgatctcgtgcaagttgcttaggtcgtacttgtcgatgagagtgctcttagcgaagaagctaaatagtgtgggcaccagcagggcagattgaatcttatagtcttgcaagctgcgcaagaatagctcctcctcgaagcggtacatgagcacgacccgaaagccgcagatcaagtagcccagcgtggtgaacatgccgaagccgtggtgaaatggcaccacgctgaggatagcggtgtcggggatgatctggttgccgaagatggggtcgcgggcatgactgaatcggacacaagcggtgcggtgcggtagggctacgcccttgggcaatccggtactgccactactgttcatgatcagggcgatggttttgtcccggtcgaagctctcgggcacgaagtcgtactcgttgaagccgggtggcaaatgggaagtcacgaaggtgtacatgctttggaagccctggtagtcggtcttgctatccatgatgatgatcttttgtatgatcggtagcttcttttgcacgttgaggatcttttgcagccctttcttgctcacgaatacgacggtgggctggctgatgcccatgctgttcagcagctcgcgctcgttgtagatgtcgttagctggggccacagccacaccgatgaacagggcacccaacacgggcatgaagaactgcaagctattctcgctgcacaccacgatccgatggtttgtattcagcccatagcgcttcatagcttctgccagccgaacgctcatctcgaagtactcggcgtaggtaatgtccacctcgatatgtgcgtcggtaaaggcgatggtgccgggcaccagggcgtagcgcttcatggctttgtgcagctgctcgccggcggtcccgtcttcgagtgggtagaatggcgctgggcccttcttaatgtttttggcatcttccataggaccggggttttcttccacgtctcctgcttgctttaacagagagaagttcgtggctccggatccggcggcggttacgaactccagcaggaccatgtgatcgcgcttctcgttggggtctttgctcagggcggactgatggctcaggtagtggttgtcgggcagcagcacggggccgtcgccgatgggggtgttctgctggtagtggtcggcgagctgcacgctgccgtcctcaacgttgtggcggatcttgaagttagccttgatgccgttcttctgcttgtcggccgtgatatagacgttgtggctgttgaagttgtactccagcttgtgccccaggatgttgccgtcctccttgaagtcgatgcccttcagctcgatgcggttcaccagggtgtcgccctcgaacttcacctcggcgcgggtcttgtaggtaccgtcgtccttgaaagagatggtgcgctcctggacgtagccttcgggcatggcggacttgaagaagtcgtgctgcttcatgtggtcggggtagcggctgaagcaggccacgccgtagccgaaggtggtcacgagggtgggccagggcacgggcagcttgccggtggtgcagatgaacttcagggtcagcttgccgttggtggcatcgccctcgccctcgccgcggacgctgaacttgtggccgtttacgtcgccgtccagctcgaccaggatgggcaccaccccggtgaacagctcctcgcccttgctcaccatgtccacgccgtcgtcgttcaggttgtccttgcgcttgctcttgatgcagcccatggtggcggcggtcaccacgcgtccgcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaacagctggccgccaccatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgattcgaaagccgcactcctcataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaggccgcacacctcaggtgcaggctcgaaatttaaatcctcgagggccgcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcctgcagatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 2 - DNA, 27368 bp, Homo sapiens
accagcctgcgctcctggcgccctcatgtcttcacgggactccccgcgccggttgacgtggtgtctcgttcggatctccaggcaagacctcagctccggcagcagcatcagacaaaccagtctcgtgctcccaggcagtgcgcccagaggaggcgcagagcgcggcagctgccgctgagccgcccgcagcgccccgggcccgcgcagccccagccaagagcgcgacgcgcgcagccctgtcagccccccacccaaagcagcggcggctgctcggacctcggcttctgggggtgcgggggcccggcgggagagttgccggcagccctcgccctgttggcggcggcggcggcgggagtcttggccgccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtaagtgagggagcgggccggggagcagggagcgggtggtgcctgcgtcgcgggtgggtgcggccggctgaagggcgccctagtcagaggggtgcgtctggaccccagagcaaacggtgagagtagaaagtggaaagactagagggaacctaagctatgcgaggagagagaaaagtagaaccggagagaggccggcctttcggaggggcatgttgccttttccacgtgggcaaagatctgtggagccccagatgcgcctggacccggaatgcccaagaggggcagtttccttccccgactcggccaggtgctcttccgtcaagtcacagaacgattcagggcttgggtcatcttcgaacccaccgccgtcgaaatgtgtcctctctggacgtgcagagatactatcattgccaagagtgcaacttactattgagcttgttttgatctgtgccaaggaaaagcgcaggccgggggaggggctgctgcaaaggtgctgagaacctaaaaatggagggctcacctcctccgggcgaggtctggcaaagctacattcctcacccggttgcctgcacgttcctaacgccctccaccccaactaaccatccccgtcgaggttaggagaggggctttcttgccagtcctggattcaaatctctgtctcgaacgtattcttgagactccactaggccattgaggttattattttaatgacccctttttctcctgtactctgtgccgccttccaaaaacaccccaccaccactgccactactttacaccacctcgaggctaattccgcactggtggtttgtttcctgccccaacgaagaaaaacgaaccctcttcgggctggaaacggactttcgcgggttcccgccagcttcctggggcccagctagtttcggctttggtagaatccgccgccaggacccatgtgcgcgcccccggggggcttacccatcccagagctcgcgacccttctgctcataggcggggacaaaggcaactcttccggcccctgctgcggagatgttagaacgcgctcgactcttgctcagtgtttggaatatatgcttaggtatttcaaggaagagatgtttcttccaacctcattcacgcacccagtcccgcaaagccgtcaaagccggcctcgaccaaactcgtactgtttcgctgcgccttcgctttgtcagggaaggtggcgccttttcaacaagtaacaagtgtccgatctctgggaagtccgccgtgtccctagccctttcccgctagcaattcgggtgctctcgggcctggaacctggccctttaaacagaccgctttcccagtttgcctgcgagaagtgtcccaaattctttttccttccctcatcttgggccccgcagttatggggggttgaggggagaataagtcctctgtcagtcaaggacacgaagcatagttaagaaaagcaatagatcttccagcaatttctgtctctacctccccccgccaaaaaaaaaaaaaaaaaaaccgagtttatagcaaacccgtctccccgccccaccgcctcctttttctctctctgagacctggatcttgaaggcaaagcccaaacaaacctcccagcctgccaggcacaagaaggtgcgggggcactgacggcgggagcgctgcctcctgcgaatctgtcacggtgacgcgtggaaggggtgagggatggtctgaaagcaggttgccaagaaacccgctcaggtgaagtggggagaggaaaaccagcgagaatggaggggtgggagcctgaaaagcagagatgccagtaaggacctccacaggttcccgggctaatgggatttcgggaggctcgtagagggagtggtggttctccggttttataactaaaatgataacacagcctccgggccgcgtgggcgcgacacggctcagacggtttctggtgccccctgctgccgggaactcagaagtgcctcgcggcgagcggctgcttctgcgagcacttctgtggctttcctgggcgtccttgcctccccaggcctggggacccacaggtgccggcgcccacggcctgctgcttttgccctgccgggacgtggtggtcccggacccagaaggggaccagcttgccaggcacgagaaatggtgcgcggtcacctggcagagcagagcccccgtcacttgaggagcagagcttggttctcgcacgtggcggcgcgtaaagcagtttgcaccgcgggcaaaggcccatggttgaggagaagcgcgtcgggcagatgtagatgtgttttgggaaagcgatagagagacaaactgagaagccgaggggtggagcccacacggactctaagaaaaacgtcctagtagacgctgtgtgtgccgagtcccccaacctccccccttttcccccgcccagcaaataccgagaacgaaagagcccctcggactggagccggtcccggcggcgctcagagggaagaatgcgccctccctgtcccgggggaagaggggagctcgatccgggcgctggcggcgctcggccgagggcgggaaccgcagccgggagcctcctctggcgacgcggggagttttccactgtgtagtcttttgccagcttcgccacacttctaattgaggatctccacgtccaatttaaaaagccctccgtcggccaagcaggaaaaaagggaaaaccctcttaagacggaagggaaatctagtgcctctgtggcttcctgctggcgggcaccgcgatttggaatggcgcattaaccccgcgcctccatgctcctccggtcggaccgccagccatttcgcacgcacccgggagctggcaccggcggggggcttgaggggggaagctcgcgttccccaggtcctagctgccaaagtacctttctgggctcattttgcatggcctggtgcagttttcctgtgtctgcacatcgcgacccagaacctagctttttcccgagtttgcaaaccagcccgcgaggcaagaggcgctcggtgctgcaggtcactaggagtttctagtccctacaccctctcgagcccaacagctgcatagcgaacaaacagacgctcaggagatgtaaaatgcattagtctctgcagaggtgggaggtggcggccgaattaaaggcttccccgggttgcctgcaccgggacagggagtggggttccgggtagagatgaggactggaaccctggaatggaggcgggggtgcctgtgaactaatggctaggaaaggcactgaggtttttgcattaacctgggttttgcatttccctcccgcatgggaaaaaaaaaatcgagtttttcctaagaaccgttttatggagccggtcgaggggataggataatcgactgcacgaatctcgcagattccgcttgaggagattctctctaggtcactagtgccctggagacgaaccctgggattaggaaggcactggataacactgcaccccaagatgcctcggttcttccccgtctcctcattggttggaactcgcccccaaatttacagcccctttttctaacccacccccagctgcgtggccctcctttgtaggggtgtgaggattgagaagccaaacagagcccacctcgggctgaaaagagctgaaccccctactctgcgccgtaccacggtctaggccttccagtgccagagcacctccaaagcgtccgagactgggtacagtcgtccaggcgtgacgggggcgcggggagccagtgactcctctgggaggggaagggattagggccagaatctctcaaaggtgcaaaaatccagtcaagagagggttttcgggtataccacggaggattaaaactttcaagacaaatgcagtctttgcctaacagcaatggtaaagcatttactgagctcttactacatgttccgctagagcttgtcatgcgtgatttaatgttcataactttaagaggtgggaggagtattttaaatcgaatcagtgctttgggaagttaagtaacttaaaccagatatcccatacaggccaaaagtctccaagtccctgctaacttcttgctctcgcaacagaatacctatttaggtgggaagaatgaggtgtgggcggcaggctgggtgagtggtgcccccgagcctgccctcgactagccagaagcccggttgggacccgaggcaggggaatgcgcttgattttatttccaaagagaaacaccgtccttgcttgggccgagggctcgttcaggggcctataggagctaccgggacaagaaggggaggtctctggttggggtggaggacgaagggtgggaactacccgattgccccccaggaatgggggatgttgcgcaccagtagaggggactggacaggaatcgtggtggtgggggtggggggttaactggaggggacagcagccctgcttgaaactctgacccctaagaccgtgtggtggagaagggcagctgcaacctgaaccaggagtgcgagctgctcctggggcgcgctgaggagggagaagcgaactggggacttgcaaggagggcaggagtgcccgaggagccgctggcctgcagcggtgccggaggagggcggtgacgaggttggagaggggcgcagggacccgcagggagcccaggcttaacgtgcattctgcggttctctcccccacctcccgcctgcccgcgcaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactgtaagaaccgttccctccccgcgggggggccgccggcggaccccctcgcacccccacccgcagccagccccgcacgtaccccaagccagcctgatggctgtgtggcctaccgacccgtgggcaaggggtgcgggtgctgaagcccccaggggtgcctggctgcccactgctgcccgcacgcctggcctgaaagtgacacgcgctggtttgcccagcacagaggggatggaatttttatgctgctcctttagcattctgatgaacaaatatcctccccaccagcaccaccacctcagaaacacacacacagctgtccccttttctgtttcctcacctatacacactcccagtttttctttgcttccaaagccctttatctgtgtgtctgtgcctggctgtgcttaattctgagaactattgcactttcatcctaaactgcgcctgcaaggcgagaggccggcttttcacaaaagcaagccagaggcagagaaaacacagaagggcctccatttccagaacaagcgtctgggtaatgtcaaatctgttcagaaaagttcctctgttcagaaaagttccggttctagaaagacttaaccataaatagtgctggctgggactagggacaaagactgtagctcactccacgtgagacaatgctaactcttgagaaaaaaacccaggttattcttattagaaaataagtctgtgatttacctctcaaaaattaacatgttttagaagcaagtcaattagggcatatcagctgtgatgtgatctcgttttccctccactcctcagaagcttgttgcatgaagtgagagaggctacattacatgtgatagaggctgttgcagaggcataatgtcaacaaagatagcaatagaaaatttcacacaaccccaaaataaagcaggaatgagctgtgtgtaactaggataccaccctggctactcttccctccctaacgccacccccagaattatccctctagtaactttgcgcttgaaacttactatggtcataaccacagtagtagtcctgcagtagtcagctgtgcgctgagtcccccaaacccaatggccttggctaggaaacaacccagtaaatgcaatgtgcttattttatgcttaattatagcaacaaaagcaccttcttccacaagtcccaagaaagatagaaagtggatttttttataaggcaaattaagacttcaaaaggaagttagggaacatggttaatcctttgaaactaaagtacctgttccaagagtgtctgtcaattaactgtataaacctactttaaaggcttttatttccatcacaaatttaatgtatataaacgtttctccaacagaaactttaaaatgccccttagtgccgggtcttccaaaactggctattcattcactcaaacggtctagacaagagggacaggaacacagtggcagataatagttctctaacccagacgttgccaagagaaaaatgactgtgcctgattacaaagctgattaattcatggaggtttaattgctgtattagtgccctgttcaactagattcatttaattctggctcaggctcctggctctgctttcaactaactctgtgactttcaggcaagttactttagagctcaatgaactgtaccataaagagaagcctttctactccacaatcccagtggctatgagatttgccccttggcctttatgtttgtcctaaagtcctttgggaagaatctcctaagaagagattggagtcacctggggaactttaaagactactgatgctaggtctcaccccaggagattctgatttagttggtttggggtgtggcccaggcatctgagtttttaggtgttccccagatgagtctaatgtgcaaccagacttgaatcaccatggcttgataacacccaagaaaatctttggctcatataaggtacaagtagtcacaaagcaagcgagttaacacagattgcagggacaagggcatgttgtagggaagagggattgcttcccttttccaaaattgatgctgtgtcatcagtgaacaagattctcactactctatttacatttgaacagcaaaacacaccccattgtgttttgtctgtgggaagacccaatagatcccagaggaaacctgaaaaagaaacgttcccaaagagaaactgagctgcattctaagccaaattgacttctttccagatatgtttgattcggtagcaagctgtcaagtgcagggaaggcaaaataatgacagtatgcagactttgaacactcaaccagtgtcaacacgcctctgtcacagtgctgacatttatatcctgcactgtacatggtgcaactggttaaggcttatgtagaaaaacattaagtcaccacatttcatttaaaaatagaaagtatcataaattctgattcctctagttccatccaaatacttgtaacttaatgattgagcaaaagagctttcatgccacattaacgtcattctgcgcttttcacaggaggaccagaatatacacagtttgcacactcacctttaagattgcatgtgttccgctggttccaatgtaaataaaacatccagaattctattactagtatgccctcagtgtgagataaccaaatggaattcataattggccaacgggcttgcctagaggctggctgtaaagtaaatggctcaggactgcctcctgtagcaacttctagcctgtctaaactcaaggacttgtgatttgacttttggggtacccaagacttttctcttttgctatctttatttgtttattttgctttttgccagtattttgccagtattttgctttttgccagtttccaaagggcaattagagcaggcatgaggaatctgcaagttgaaacttgcaaatgtcatagcattttcgagctgaaaggaaacccctctagtcctgaaacctaaagaggcaaagcaacttgtctataatgcaccacagttccatgatcacaggaggcaggtctgaagctgggaccagagtttcccccaaactactgaaatgtactttatatgggaaggggaggatactttatatgggaggaaggtcttgaactaatgatttagaccatggtgcacaaggttgtctgaatttattgaaggttagttcatcatagtcatattccttatctcaactctaaatgtatttaataagtaaaagcataaaatgcatgtggttttaaaaatttgcatctaaatgcacatatacattcaccaaatagttacatgtatgcctgtctgtgccaggcactgtcctaagtgctagggataccttgatacacctaataaacaaaagtcgctgccctcatggaacatagcttctagtaaatgcacatgtgtggtggtatgaaaaaatcaacgtgaaagaacctcagtccagtaaggtgggctgaattttttgtgcaactggaaaccaactaaaatgacattgagcatcgttaagttgtaattacaagtaattcctaatttagtataatataatgggattatattctaccagtttggtagcagttcacaaaatttcctatggaaacaattttttaaattaagctatcaagttttcaggctagcccacaaaaacctatgtaaaccacgatataactgaactgtcattcacaatattactaagtgagagtctctgccctggggggcacatcccataatctgcaaaaggagggcacagaacaagaaatcagggaggtttcagcaagcaagagctcaaggtaacacattcagaccagactaggatttggaccccagccttgccacttaactgagatgttgagctgttacttaacatcccttaagctttgttttttcttccctgtaaaataggcataagagtaaatctgtctcatgggttgatgtgagggttaacttagatattgctttgtaaagcctctgctccataaatagggatcatagttgtcatttgccaccctcctgcttgcaatggcccctccctgggcacccagtgaattcacttcttccgctacgacatctgccaggatgctgctctaaatagtgggatttcagcagcacaacagagtacagcgagtgagagaaatgcaggccttgtgggcaggctattttgggccccactgttggttggtcagcacgtaggcagtccaggcttgcctgggattctgcccctgggtcagctgcccctggctcttcccaggtttcacagctccgacccttccccagtttccaagcccatatatttccagtggaatttttttctcaccaaactcctatggttatgttagggagacctcttccgtcatgggaaggagggaaccaaggcagggtaggatctttctatggaagtgacaagagggttgtgagttggaggctacctgtatggagatggagcgtatctttgtaaatgatccatcccagctggacatttaatttaagaagtttcataaccctagcaaaaaatacttagtaaactgtgagaccacctctatagagctaaggaagcttctagtgcaacaagctgggtaaatgttaagctttcaaatctaatgttatgaaatacagaatttagaatgaacaaacaccatctatctgttgtgtcttatcgcttagggactgtatgaatgaacaatactttaacaaagtaactacctaagctggaaaggagctctgccacttgggatgggtctcttggaatcaagaaactgcacatcagactcttgtaggaggcactgattctttacttgagatctgagaccaatattctcattctggccccagtctggaaaaccatttcaaactgttactgcttagagaaatctatttaaggcttaaattgatttggccagtcaggaccttaggctgacccagggcgtgagcaatgtactcaacaggttcttcttcaacagatataaaaattaaagctccctgatattttcccctcctaatatctctttgatctttgtttatttaacactcatggaccatagcaccttcatttgattaaaatacttgttttttagttaattacttaagcttaatcaggaaacaaatattaaatatctttcatgtactgggtgccatgcggcctcaaaaaggctaaatttgtaaataaagttgcttgtgatctagctgtaaactattgctaggtggatgggcggatggctggatggatgaatagaatgtcagtcagatagatatgtctattgtgtagggtttaggtgagctgtacgcatgtgtggtaaaaattcctagcagtgagctcactgggatttgggtatcatagcagggacgacttcagggaagagggaggatttgacctggtacttgaaagatgggtgtttggaagggaagggaaaaggaaagggggacatactagggagtagcatgggaaaatttgtgaccatgatgtggggaagggacagtggaacaaagaggaggacagaccagtctccaagttctagaaagaggtgctaagaagtggtgggaaattgaagatgtcttgacaggtgataccagattgtgtgatactataaaattcaggaaaggtgtttgctctgtttttgatagcagtggggcacccatggagcaggcaggtgactacaaggcgggatcttgcttcaaagctgtcttgcaggagaagttacctaaatacccagactgcttttgagtggggcctatgccgtaagcatgtcttttattttgaggtgttgtgcttttatttttaaaaatcttctagacataggcacaattgaaccaaactattagccccaagtaagtgaatatctgcaaataaacacttaatattaaagttaatgacctagcaccttttaactatccacatgatgaacacagttcttgtcctgaaatcaagtaagtctgagctctacaatagaagaggagatattattatccccattttacaggtgaagaaaggagacctagagagttgttccgggtcacaaacctagtaagtggtgaagccagaatttaaacctcagcatgttggtccgaaagccaatatttcttgactttacacagactgtgtgtgcatattagtgaattaagaaaatatagactttggcttgcttaaaaatgcactcactaaccctgaaacacagatttccaggaaaattaagcaagcaaagagaaaagagaagcagagactatcaaatttccttttggcccttttaaaatctccatttgggctgcggtaggcttaagccagtattactaatgactacttcaaagtccagatcaggatgtttttaagaagagaaacatgaatttctctaagtattcctataatattgatgctttttgcaatgagagaaggctccctaactctttgcaacaaagcaaggtctcctcaagtgcttggtaggcagacagcattcgggaggccttgtgggagactctggttctcagatctctcccattctgcccagcgaggggatggcatccacataggaacctagtgtgactgcacgagtgccagagcgatggcctcagtgggaataggagtatgcgtaagcagacttgggtcaccactgggaaacaactggttagctcagtagaggcaaaacccacttttgcataactttaataacaaaattgaagtagagaagcatggttttaaaacaatatggccttccaatttttttgcctgcaaacctgcaaataagaatgttgaaaaacgattacctactttgaagctttctaaaatttttcatcataggtttaaatagttacaccagatgtcatttctagccctttcaggaactgtatatatgctttaaatattcttttggcaaaactttgcacctgcctatgtagtttatttagttcatggaaaatgatcaaactaatcagcccaaatcaattggctttttggtcagaaaaggtctgacttcattttcaattcaaatgtacatgttaatatacaccccatgacaactgtcacaattctggatcctaattgtaaggtagagagatggataggtgaacagtcagataagcaaagaaagcactgaggcttcccaaggggcctggccaggaggaaatatggagcaactggcttcaggatttcttggtttattcacaaagttatctccctttgcaagtgtttgtagcacagtgaacacccatacatccttcactaagattaacctcttttttagcattttgcctcgtttttttctgaaccatttgagaatttgttgcaaacatcagaatgttaaatatttcagtatgtaccttctaagaacaaggacaatgtcttatatagccacaattcagttatcagtctcaggaaatttaacatggatataatactgttatccaatagatagtctgtgttcatatgtccccagttgtagcaattatgtcctttatgttttgtttttttttttttaaaacccaggaccataaattgcatttgactgtctggtatctttatacttccttaatctaaaatgattcccaagtctttttttcccgcatgacattgacatttgtgaaaactctgggctcttttgctagctgtttcccagactacccttcagtttagatctgtctgattatttccacatgattagatgtagggaaacatccttggaatatcatattcatggcactatgtcctcacatcaggaagcgcccaatatcagtttgttccaaggagccattcatgcatgaatgcctttgtatggcgcctgaaggtgtttatccctgggacaggccccacaatagaatggctagataggtgagaagcacaccgtctctcagtaaggataaagagggattggaaaaggggacatgggcaaggagaaccacagcaggagcatccgcaggcagaccagacattttaggacatggcacatgtggaaattgaggaaatggacatggattccctttcaaccacccataggcactgcagaaagtcttcctgtgcccccagagggccaggtgcctcagtctgaagacactgctctaagccatagtggacacaaagataaccctcatgggctctggggtctgctgacatttgcatgtttagttgtcaggcattttgaggaacacggaagagctcacacagcctcccaagacgggcctgtggcttctgcccacaacccaccatgccaggggctccaggccctgcacagaggtctctcctctgtagaacatgctagactaagtagggaccagtgtctggatccccttttgggcaatcttgctttgtctagagactgtagagttggctttacactgctgccttctgtgcaaatattgctgaaggaggatcccagatgtgataaccatattggcctacattaggcaccatttgtgaaatactttctgtggaccaggaactgagctgaaatcttttcttatgttatcatattaattttgacctgaaggtaaaggtgtgactagccccatttgaaagatggggaaattgaggcttacagggttagcagcttagccagggtctggacacagggcagcctgagtccagcactcacacttgtcactactgcacaagatcacttcctgccagcccatgacactcagatgtcactcttctgactgcatcaaaaagtcatcaaggaaaaaaaatcagggaatgggtttggcaggtaaagttgttttagaatgataaccgtatctcattacctgaagagtctttgagattcccgtaaattcactcactgggggtagaaattccctcctcatatctgaccacaagaatctaccaaacaaattcaaatgataaagaagatttctttcatcttctcttagtccctccttcttgttcaaatcactgagacccttacatgccttttacctactgctcagtgggtcccctgggaagagctgagggatgctgagtgcagaatcctgcagggtcctgcagcctctcaggctgggggcagggcttcgctgaaagaagaaaggagagactccaccacccccacaaccacctgcctctgataacaccacagggatgatttgccctagtggctttttgtgcacttaattttactgggtaactttctcacccttccctgttaagttttttataagggcagtagcagacctcctggtgtgtgctcattagcttgtggtgtttattaccgactctggagtgcttttagctcacaccaggtgttcatggattagctaacaaatgaacaccctgttgggtgttttcctgacttaaaagctgaagaatggacactttccacccagggggactgtgctgtattactgtaaaattattagcataactgtaataaaagcatggaccacatacatagaaatcagagcaaggtgatcagaacctgagtaacaaaggaatttactgtctgtctctccctgagtggggttttctggctgtttgtattgatggagtaattttcagtccatttattacaaatttgcttagttgagttgtaggataacagtttaggatatagtacaactagtatgtgcaatgtcattcagagtgggtggagatggtaaataagatggcattttgatgggcaaagtggcttttctaagtacacccatagcttctttttctatattctaaagtgatttgcattctggttggtctttttcttctgccttgagagaatccagaaatgcttttttaaaacaacaaaacagtggtgtttttacaacgcaaatacttttcaaataaatcgatgtcatgccttactgtcaacaaaccactggtcctgaagagaatgcactggtagctctggaaatggtcacaatgacttagtaaattgcctcagctggtaattgtttttaggaaaatagatgctgtggacacttctgaaagttaacccaacagcagcctatgatcaggacggtctaccaaacactagatgaattctgtgtccaaaataggaaagcacggaaggtcattacataatgtaagatgcatcagcattcagtgcttactgatctatgggccttttttaaaaagtagttcaaataagtgtcaaagtcatcactttgaaataggagcagataacaaaactttacagaagtttttcagagaactagaacattctcataaactcacatttagagtccattctcatggactgcacattttagaggttcctgaaggtcaaataagaacaagagttgacagcccagagattggcttcaaggacaagctgcttggctctcctgatccattttgtaccacttgcagtgggcaattctagccttggagtcataagctgggtatgacctaagcatacttgaagcagcaaaaacagaaatgaataaaattgagattcgaagaaaatggtaaattgagtgtttgacttttgggtgatggagactgaaggaattgtagcgtgaggctgtagtgtgtcctctcaggggtcatggggcccatctctatttttacagatggaaactgaagtttaagaatgccttacagtagaatctggatgctttcatatgcagatacagggccctttctacacatcttttacctctcttaaatagggcacaggaagatgacttgatgatttaagagaagattgatgacggtcatttcaaatgtagccgagacattcagccaagaggtaaactgaagaggtcaagcactgcagagttctaaaatacctcctgtggggttttatggggcccctgatggtactggctgagctgaatgctgctggggcgtcagccagaggtggtctactcccttgcagaggtgactgaaaaacccgtgtctggccacactttccagccaagacttagactctccacactttagcattttggagctggaaaaggccccagagagcactgagttgacctcagaagggttaagtgactacttccaaggtcacgcagctgatcagggactgacccaagactggaatccgggcctctcttgtctccaactctgcagcaagagcctggtcatttggtgccagcatgagttggaggagcttccggagatggggcctctctgtctggatctgctgctgtgctggctgcggcttttccggttttaactgggaaatcgccagagctgtcttagcgtgatatgcaagaaccaggacacaggagaaatgcccctgagtagcatggcttttcctttttgggagacaatttactgtattctgtggcccatggcagcctaactttaggatctacttagcgtacctgagttcgtactgaatttttcaacagaaagtatgtttctcacctcctgtgctgactttggtaaatgtgtacaggtgaaaccagcatgtcttgctctcgtctcagagtaaattcccatctgctgcaagacttgaagagctcagtggtagtagagtttttactttaaaacaaaaagacaacaagtttgagctttgaaactgaaccaccaggtccacatttattagtcctatggacttcaccaaatatttgtcttctctgagcctcagcttcctgagctgtaaaatggagatagtattcaatttagccttgaagggaggttgtcaggtttaaagttaatgttgtgtgaaagtaggcagtattccacctttacactggtaatgttttttaagtgcttcaaggaagctcttatctaagttatagcctgatgaaattttgtaatgcaagaagttttacaggttaaactcagccatgtagttcttgtaatgatattccaatattagtgaaagaaaatcatgtttgtaacatatagaaagataaaaataacttattccaaaacaatgacattcattgggactcttcttgagaagttgtatcaattttaagttgatcttttcttttcatataagtatgtatatgtgtgtctctgtgtgtagacgcatatgtatgcacgcatgtgtatgtgtatgtgtttatagatcgagagagatctctatatttttcagatacttggcctgtagagcaatttgcattttacctgcaatataggtaggcaaagaataccagattaaatgtctcccaccattagggggttttccattttcctcctgtctgaagacagcctttcataggaaccgctacatgtcaggatggatggcaccgcacctcggcagccgcaggtgcagcatcctgtggcctcatcctcagcatcctgccccatcccaaaactgagccccaccatctggccactggttcctgatgtatctctcacttgtgtggccttggctcaggcagcagctcacccctatgggtcccattagccctcacccttccaggcacagaagctggacactatagtgaagtagcaaggctgttctccccacagcaagaaacccctgagccttctctctggggcccctgcatcagccaagcccggctcatgcagatctcaagcctggcctggaagacatctcctgaacaggaccactgtgtgtactgaggtcaggccccctctctcctgggtccctctgagcctcaaagaccttccttccaccttctggataaggagttgtgtcctcaccactttgtccctcatagacaactttgtgccatctccctctcaggcccacaggttggggacttaggatgacttagaatgaaagtcagcttagactccctgcctggccaggctgaggaggggacaaccagctacaagtagaaaaatgaccctttgattaaaatatagttaacctgtactgttttaagacaaatctggcattttacgaaaagactttgtccactcttgccagcttaaccaatgtaatgttttggattatgtaattttgagttcagctattacagggaggacttggagccctcaccattgttcctattaaaactcagttgactttacaaaatagtcactatctcagtcctttgttcctgggatgacattactattttttttccccataatcgagggcagcaaagtctttgcggtgaactgtttgttcccagagttgtgccccggtacaaggtttcatttctttaagtaacactatttatacaaagaacacatcaaagttaaaatattttaaatagaataacaccaaaacatttggttgcactctgaaggatagcttaaatgcatttgagcactttggatttccaaaaactttatattttagatggacatgttttccaagatatatcctccaagcaggtctgggaaatgagcaaagatatgacctattatttttttaatgatgctcctcttttgtggtttgcaagtatttcactattaaagttaataaatgagggtcatttgcatatctgacattctgccaagccttttaaaggatgaagacagagatgcgccagtaagtcttaatttagggaacgaatccaaacccagacccctcttcttttccctcagctcagtgaattcctggaaaatggaggcagctgtgggcatttcagctcatcaccaggagtttcttgtctgggctgggtgagaggcctctgcagaagaattaaggacaggctcagtgaggctgcccagcatcctctgcagaggagtatggcgcctaatgcccccaggtgcctcgcatcgataaattgaggctggctctaagaatgaactcatttagtcggaacatgcaggcctatttgccctgtggtttgaaaaatacaatgttgccctttcctggcttccagaattcatatccatgtaaatttttcagcaaaaattttgttgtttcttctatttttcatgactatgaaggaaaaaaaagccttcagcttaataagagttgcctatggcctgaacttggggtttaaataatatttccacattagcaacaaaatgtgaaggagatttcccatcaaaggaaggaattttcaaaagccacatgccaggaagactttaaaaaataataataataatagactttattttttagagcagttttaggttcacagcaaaattgaaaggaaggcagagagttcccatatactccctacccctacacatgcatagcctcctccattgtcaacatcccccaccagaatgctacatttattatagttgatgaacctacattgacacatcattatcactcaaagtccacagttgacattggggttcactcttggtgctatacatcccatgggtttggacaaatttgctatgacgtgtattcactatttacctgaaattcaaatacaaccaggctttctgtattttacctggcaaacaaagcctgtgacaaagccatgtggtcaaaatgtcttaaaaaggggagaaaaatgtttttcaagttcttccaagtatcaagggctaaaaaagaacactataagtgctgattcaaatccttatgattgtaactctagtaataaaaagttatatatcatatagattaatgagtctctaaaccagcatgatttaaacttctgtgactctaatgttttcctattagcttatattaaaattttagtaatgttttaccctcatttctgattaatcttcagttgattgttaattgaacttaactttctatatgcccaggttcctaaattaatgacttatttagaattcttgaatagagcctatgatgtgagtccttttgtaaaacagagtctctgttttttaatttgaagactcttctgtattttgattaatcactcctaatagatctgtttcataagttccgatttatatacaagcatgtttttttttgaaactgggcacatctgtatttatttcctctttaatagcttattattgaaagaaatcaccaatagctaaagcagctcatttttttttaagtcaattgtttctgtcaaccaaagtgatttaatgttgttgttttgttttgttttgaaagaaaacttcctaaagaaagttcagtgagaaagcagtacaaaggaaaggacaaattaagagcactcttggccaggcacagtggctcactcctgtaatcccagcattttgggaggctgaggcggacggatcacctgagatcaggagttcaagaccagcctggccaacatggtgaaatcccatctctactaaaaatacaaaaattagccgggtgtgatggcgggcacctgtaattgcagctacttgggaggctgaggcaggagaatcacttgaacccgggaattggaggttgcagtgagccgagaccatgccattgcactccagcctgggcgacaagagtgaaactccgtctcaaaaacaaaaaaaaaaaaaaaaaaagaaaagagcacttccctgggcccccttaagaaagcattactcagggaccagagagctttaggagacccaggggaggtgggcactgagagttaatgctgctgcaaagctgcagaaagctgaggaggaaaggtggtttggagacagtccttcagctctctggtgggcaatgactgtgccactcagggcagagtggcttttctgagtaatgtgggctgtttactttggagcatttcatttcataacgatgcagttttcagctaaagtcccagagtccccttgaaaaattaagaactctctttccgcctagtaaaatgacccgactcattcagccagcagttaccgtaggcctccacagcctactcaagcacttggagatccatcagtgaacaagcaggacagagttcctgcctcttggagttgacattcttttagagaagacagactataaataacaaccctaagaaatcagttagatggtgtgttagaatgtagcaagaattatggtggagggggaggcgcagggcaggggtatctggattaggggctggggaggaatcttgttttaaatagggaggtcaggtgggacttgtggatagtgacatttgagcaaagacttgaaggagatgtgggactagccacagggtgggagacatttaatctggtattctttgcctacctatattgcaggtaaaatgcaaattgctctataggccaagggtccaggcagagaggggacagccagcacaaaggccctgagttcttgactgtttaagctatagttctcaagggatggtgttgcccctaggggacatttgcaatatccagagataccttgatctgtcatgactaggatgggggatgctatgggccttgtattagtctgttttcttactgctatgaagaaatacctgagactgggtaatttataaaggaaagaggtttcatggactcacagttccacatggctggagaggcctcacaatcatggcagaaggcaaaggaggagcaaagtcacatcttacatggcggcaggcaagagggcataggcaggcaggggaactgcactttataaaacaatcaggtctcatgagacttattcattattaccagaatagcacaggaaaaacctgcccctgcgattcaattacctctcactgggccccctcccacaacatgtggggattatggcagctaaaattcaagatgatatttgggtggggacacagccataccatatcaggcctctcctggatggaagccagggatgctgccaaacatcctacagtgcacaggacagcctcccacaatgaagaatgacccagccgaaaaggtcagtggtgctgacactgagccccgtgtttgaggaatggtaaggaagccagggtagctagacgggagggaacaagaagagaaacaagagaggaggtcagaggtaataaaggctggaggagaggggcagccaggtcaggtagggcctttagggccagtgtggatttgggctttttctctaagagtaacagagagctattggaaggttttgagcagaggaggttatgttctgcgtttaaagtatccctctgggtgctgggtagaaaatagatcgaaggtgggtaagggtagaatgaggaagcccagttcaggggccactgtggttatccaggcaagaggtgacagggcttcagccagtgtggaaacagctttgtgatttccgtgcagctgatcctgccaatgctaaatttcagatagcaactaggcattctacggattccaatcaccagtcttttcattattatatatcatatgttttatatattacacacacacacacacacacacacactctacacacacgtggttaagttgcttttgctagattttgttctataggagggatacctgatatattttaacaattaaataggttaggtttatgacaaaacaatttgacatgggttagacataatgtaatctttctatgctttaatacaatcttggcaaaaaacatttactcttgctgacaactcttctttggtttatttctctaagccacttagaatcctaaacatactaaatgacctgaaaaatgaggtgaggggggcataagaggcctactacccaagaaacaggtgtttggattgtttttccccccgtttagaaatttattacattgttccattagttatttttctctgctgacttgattggccaaagcaaacactgaaatccggcagaaagcactaagagactgctgatttatactttcaccaaaggtctcagacatcttagtgtccctgcatccttcctccatgtgatctccctaatgagcctcttgtcctgcttgcagccagacagtgggagccatgtgacagcttgcagctgaggctggacctcacccttgggggcagggctgcttctatgcacgaacagcatctcatttcatttattgctgtgatacataaatgccattttactctatcactggacttttttgttaaagtcatgctgttcaaaggaagcggtaaattagtctttttgttagtaaaatataaactgttttcctgttcctttatgatttttaaggaaattttaaactatagttgcattcctgagctcttcatttgttctgtcagaatggttcttgtcccactcagccacacagaatcaaagcaagtttcagggaagagcactcagtattccagatgaaggtagccatagtggaaggcctcagttttttcaccctctaagacggtgtctctcaaagtttagtgcgtggatctttggtgtgtggatcttttacattagaattccttgaggcttgttagaaatgcagattccttggtccctccccagaccaaatgactcattgtctccaagggtagggccaagaatctgcatggtagtcaatatccagggtattttcctgcatattcacatttgagaaccactactctaggttgtgtgactaggcaagaaaaatgcccctagaagattggccagctcacagcaagctctgcatggacttgttaaaaatggtgaatgttattcaaatgaaaactatgcttctaaggatttgttttcttcgagaaagtatgtcaccaccacattagtctcctcttccaactaaatcatctttcttctgtgcatttttgcctctgtttttggggattacagtggtcctatagcttaatcggctgatagttttgctgtgggtccaatttttgctgactttaggggggcactttgatcttgaagacggcttgaaatgatcattttgtctcatgtgccattttctcttttctttttgaacagcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 3 - DNA, 4171 bp, Homo sapiens
agcctgcgctcctggcgccctcatgtcttcacgggactccccgcgccggttgacgtggtgtctcgttcggatctccaggcaagacctcagctccggcagcagcatcagacaaaccagtctcgtgctcccaggcagtgcgcccagaggaggcgcagagcgcggcagctgccgctgagccgcccgcagcgccccgggcccgcgcagccccagccaagagcgcgacgcgcgcagccctgtcagccccccacccaaagcagcggcggctgctcggacctcggcttctgggggtgcgggggcccggcgggagagttgccggcagccctcgccctgttggcggcggcggcggcgggagtcttggccgccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgactccggctccagagactgctgtgtattgcattcctgctacagtgcaaagaaagggaccaaggttcccaggaaatgtttgcccagaatggaagatgaggaccaaggaggcggaggaggaggaagaagaagaggaggaggaggaggaggaggaggaggaggaggaaggcagccatcatgggagcctggtagagggagatccagctacagacaactggacaggagagagagaaaacagccctctggattctccaggatggcagccgatgtcactagaagctcagggctgatgttcctggttggctattgccaccatttcagctgatacagtccaccatcactgattaccggcgcggttgcggtggatgcacttgaaccaaaccagtgtatctcctgtgatttgttttcatgtgtccgaagacaccagggaaacagagatcctggtgttgttccttgttattacgttttactgctgaaagtaagaggtttatttttctgtcactcagtggagacataccctggaaaggagaggggaaaaaaaaagcaaagatacaagagataatcacctttgcatttgaaagttgaggcccgaggtttactacaaccagcatttttgccaacggttggtgattgatttccatcacggtgtgtggggtgggaagaagttggctaggaaccaaaaaggctgtgctcatgattaaacacaaacctgaaggtatttcctttatgtccttggaaacaggaaacgagttgtggttttcgccagcattcttgtaggagagaatcggggaaggccccgaactgcccccgggcagggagagcccctcaggcctgttggtttacagagagacagatgttacataaccagctccgttgatgcgtggtcaccagtgaccagagaagctactcgatgcaatgcatctgtttcagatacagaaatatagagaagatatttattgaaatttaagttattgttatttattaccgttcactaatgaatttctcttttttcccttatttattaaagtttcttttcaaaggtgccaaagtatatgtgctcgcaaaatgcaaagaaaggtgacaaaaggaaatttgaattgggaacaagggtccatgcttttcaaagtattaaaaagttttttgccaggcaaaaatcacttactttacctttttaagaaaatttgtcattaattttccccagatttcagcatttttcccaatttttatttgtggagcatctcaggcaagccccctttcctggagcagcgtgcagagaccactggcacttgactttatttcttccttgctccattgctgaacagaaatgtcgtgggctccacttcctgttgtctttaagctcttagtcccctccacgtatacctatctgtactatgcataaccatatgtagaaaaggttcagttccttttagtaggtagtcctggatttaatgctgacctaaaagtaatgtcgacaatgctgtcaggtagctgccgttctaccgactccctccatccctgcccacccactgccctcccgagaatatgctggctgcccagtgcagcccgggagacacaggggccttccagaggtagggtctaccaggtcctgtacaacccctgggctgtcaccgggggtcaacagctgctgctcctatatacccaaacacctgacagctccctggggagcagatggctgagaagggtgctgaggaagccatattgggaccagccacagccacacacatggagcctcatacttaggagcgtgctgcctttaaatgaaggtggtcggggccagtgcagcggctcacacccataatcccaacactttggaaagccaaggtgggaggatctcttgaacccaggagtttgagaccagcttgggcaacatagggagaccctgtctctacagaaactttaaaaattaggcaggcatgatggtgcacacctgtggtcccagctactcaagaggctgaaggaggatcacttgagtccagaaggtcgaggctgcagtgagctgtgatcatgccactgcactccagcctaagtgacagtgcggtaccctgtctcaaaaaaaaaaaaaaaaaaaaaaagaggttggagcaggaggaagcataggggcgggaacagccacctcctccatgccctagattgtgaatttatcgggcagccaacacatgtatgacacactaggccctgtattacagcttgttacgcatttcataaaagggattttcattaaggagataatctattactacctaccttagtggctactagtataaaactatgacagatttagcaattaaatgaaatactggcctccatcaaataatcatagtaacaagaagcagcagttaccagacatctgatccccttcccccaaaatacccaaattcttcatggttctgcccttctctgtcctttctgctccccttgctcgcctgggaaatggaggaaaggccttccctctcacactgtcttgggatcttgctgagaattcagactgctcgaaacagtgacaaaccccagccatccagtcattcgtggagcacaatttggatgtggccccaggggcatctgtcccattcagagaaccttggcagtgcgatggccactgttcccaggcttcaacctcagtgaccccccccaacaactccccatggagagtccctgcccaaaaaagctgtaggatccaaggggtgtcaatagctcgttcccggcatcacctacacaccacaagcaggttttaatggaagcaagttgctccaccaaatccacaaaagggtaaagtttgtgatttttctttatcattgcgatcaccatctgataccgtaaggagtgcacttgtttggaagttctgacttctctgatctgtcttggtcgtttgtgttataaaaccaaagttctctacagactttatttttgtacaatatcattttgtaactttttacaaataaaaactcatttctattgc

SEQ ID NO: 4 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 5 - DNA, 11360 bp, Homo sapiens
cagtactaagctttactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctctaagggcaagggcgaattcgcggccgctaaattcaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcagaattaaccctcactaaagggactagtcctgcaggtttaaacgaattcgccctttgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtcgaccaattgcattcattttatgtttcaggttcagggggaggtgtgggaggttttttaaagcaagtaaaacctctacaaatgtggtatggctgattatgatccggacgcgatcgacgtcgatcgcgtcgtacgtatgatccttatcacaccttgctcttcttcttggggagctccacccactcgtgcaggctgcccaggggcttgcccaggctggtcagctgggcgatggcggtctcgtgctgctccacgaagccgccgtcctccacgtaggtcttctccaggcggtgctggatgaagtggtactcggggaagtccttcaccacgcccttgctcttcatcagggtgcgcatgtggcagctgtagaacttgccgctgttcaggcggtacaccaggatcacctggcccaccagcacgccgtcgttcatgtacaccacctcgaagctgggctgcaggccggtgatggtcttcttcatcacggggccgtcgttggggaagttgcggcccttgtactccacgcggtacacgaacatctcctcgatcaggttgatgtcgctgcggatctccaccaggccgccgtcctcgtagcgcagggtgcgctcgtacacgaagccggcggggaagctctggatgaagaagtcgctgatgtcctcggggtacttggtgaaggtgcggttgccgtactggaaggcggggctcaggatgtcgaaggcgaagggcaggggggcgcccttggtcacgcggatctgcaccagctggttgccgaacaggatgttgcccttgccgcagccctccatggtgaacacgtggttgttcaccacgccctccaggttcaccttgaagctcatgatctcctgcaggccggtgttcttcaggatctgcttgctcaccatggtgctagcaaccggtacccggggatcctctagagtcgactgatcctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgccgggtcggcgcgcgcgcacgcagcggccacaccctcgggcggccagcggctcgggcaggaagtgcgcgcaagcgcccgggagccgcggcgaccccaccccttccggccgagcccgccttcgccccagcccaggccgcggcaccccgggccccagaacgcacgcgcaattagcgccttgagtcccagcgcgcacgcgcaattagcgccaattcccagcgcgcacgcagttagcgcccaaaggaccagcgcgcacgcgcaatggcgccccagcccccaccgggcctggcgggggctacgccgcgcccaccctgcgatccccattggcaagagcccggctcagacaaagaccccgccggttgcccccgccccgagagcggcacccccggagcgcgcctcccgagcgcggcctcgcgcctccgaactggcgtgggggtgtcccccatctccggaggcccaggggcttctcccgcgccccccacggcggtccggttcccccccccatgcgccccccgctgcggcccagacggcggctctgcacgggcgaaggggccgcggccgcatgccccggtcggctggccgggcttacctggcggcgggtgtggacgggcggcggatcggcaaaggcgaggctctgtgctcgcggggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggacgcgtgaatcgattggtcgacagatctaacttgtttattgcagcttataatggttacaaataaggcaatagcatcacaaatttcacaaataaggcatttttttcactgcattctagttttggtttgtccaaactcatcaatgtatcttatcatgtctggatcttacccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatggtaattcgatgatccccctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtactagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgtggaattatcacctcgagaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctctcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaagaattcggatcccgtcgagaattagcttcgccaccatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctgaggatcaattcacgcgtggtacctctagagtcgacccccctcgagggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcgtcgac

(19) United States
(12) Patent Application Publication
(10) Pub. No.: US 2024/0076629 A1
(43) Pub. Date: Mar. 7, 2024

(54) CORTICAL NEURAL PROGENITOR CELLS FROM IPSCS

(71) Applicant: Cedars-Sinai Medical Center, Los Angeles, CA (US)

(72) Inventors: Alexander LAPERLE, North Hollywood, CA (US); Aaron FULTON, Huntington Beach, CA (US); Clive N. SVENDSEN, Pacific Palisades, CA (US)

(73) Assignee: Cedars-Sinai Medical Center, Los Angeles, CA (US)

(21) Appl. No.: 17/766,704

(22) PCT Filed: Oct. 22, 2020
(86) PCT No.: PCT/US2020/056896

§ 371 Date: Apr. 5, 2022

Related U.S. Application Data
(60) Provisional application No. 62/924,523, filed on Oct. 22, 2019.

(51) Int. Cl. C12N 5/074; C07K 14/475; C12N 5/00; C12N 15/86

(52) U.S. Cl. CPC C12N 5/0696; C07K 14/4756; C12N 5/0018; C12N 15/86; C12N 2501/11; C12N 2501/115; C12N 2501/235; C12N 2501/999; C12N 2740/15043

(57) ABSTRACT

Described herein is the production neural progenitor cell lines (NPCs) derived from human induced pluripotent stem cells (iPSCs). These iPSC-derived NPCs engraft efficiently into the spinal cord of ALS animal models and provide neuroprotection to diseased motor neurons, similar to the fetal-derived cells used in clinical study. Clonal lines were generated with a single copy GDNF construct inserted in the AAVS1 safe landing site, including inducible expression of GDNF expression. These new iPSC-derived NPC lines are scalable to clinically relevant production volumes, uniformly produce GDNF, are safe, and represent a promising new combination therapy for neurodegenerative diseases such as amyotrophic lateral sclerosis (ALS).

### CROSS-REFERENCE TO RELATED APPLICATIONS

This application claims benefit under 35 U.S.C. § 119(e) of U.S. Provisional Application No. 62/924,523 filed Oct. 22, 2019, the contents of which are incorporated herein by reference in their entirety.

### FIELD OF THE INVENTION

Described herein are neural progenitor cells (NPCs) derived from human induced pluripotent stem cells (iPSCs) that can be engineered to express ectopic proteins in an inducible manner and used for engraftment in transplant hosts. The claimed invention relates to the technical field of regenerative medicine and degenerative diseases, including neurodegeneration.

### BACKGROUND

Neurodegenerative diseases are a severe economic and care burden that will only continue to grow for an aging population. Amongst these diseases is Amyotrophic Lateral Sclerosis (ALS), which afflicts approximately 30,000 individuals in the US. Currently there are only two FDA-approved drugs used to treat the disease, Edaravone and Riluzole, both of which only slightly slow the progression of the disease. Promising preclinical treatments have included transplantation of supportive glial cells and delivery of glial cell line-derived neurotrophic factor (GDNF). The Inventors' group has generated and extensively characterized human fetal-derived neural progenitor cells (fNPCs) that can differentiate into astrocytes and that can be transfected with lentivirus to stably produce GDNF. These GDNF-producing cells engraft efficiently into the spinal cord and slow the loss of ChAT+ motor neurons in the SOD1 ALS rat. These cells have been banked under clinical Good Manufacturing Practice (cGMP) and the Inventors completed a Phase 1/2a trial delivering the cells to the spinal cord of ALS patients as a first cell and gene therapy. While promising, the scalable use of fNPCs is limited by the availability of the starting material and have a limited expansion potential. Further, lentiviral transduction used to induce GDNF expression in these cells results in a heterogeneous population with varying copy number and GDNF production levels. Thus, there is a great need in the art to generate renewable sources of transplant materials suitable for neurodegenerative treatment, including clonality, and cells that are capable of engraftment into transplant hosts.

### SUMMARY

Described herein is the production of scalable, clinical good manufacturing practice (cGMP)-applicable neural progenitor cell lines derived from human induced pluripotent stem cells (iPSCs). These iPSC-derived cells engraft efficiently in the ALS rat spinal cord and provide neuroprotection to diseased motor neurons, similar to the fetal-derived cells used in clinical study. Taking advantage of the clonal expansion capabilities of iPSCs, clonal lines were generated with a single copy GDNF construct inserted in the AAVS1 safe landing site. These lines uniformly express and produce GDNF. This provided a platform for generating iPSC-derived neural progenitor cell lines with more sophisticated constructs including a tetracycline-inducible promotor for regulated GDNF expression. These new iPSC-based lines are scalable to clinically relevant production volumes, uniformly produce GDNF, are safe for up to three months in vivo, and represent a promising new combination therapy for ALS.

### A BRIEF DESCRIPTION OF THE DRAWINGS

FIGS. 1A-1C shows a schematic of the pB-RTP-Tet-GDNF/memClover-FLuc vector. (FIG. 1A) pB-RTP-Tet-GDNF/memClover-FLuc plasmid that is designed to stably integrate into genome when transfected in combination with pBase plasmid. (FIG. 1B) pBase plasmid (FIG. 1C) Transgenes that are constitutively expressed or expressed only in the presence of doxycycline.

FIG. 2 demonstrates a vector map of AAVS1-teton-hGDNF. Shown are HA-L and HA-R arms, which are homologous recombination sequences that can be used to target genomic safe harbors, such as AAVS.

FIG. 3 demonstrates a vector map of pDonor-Teton3 g-2a-TagBFP-V5-nls-p2a-puroR WPRE_Insulated mpclover-2a-luc2pest-2a-gdnf wpre.

FIG. 4 shows a schematic of AAVS1 targeting of the endogenous locus between exon 1 and 2 of the human PPP1R12C gene. Initially, a recipient “landing site” consisting of a reporter/selection cassette (TagBFP2 and PuroR for fluorescent and antibiotic selection) driven by a splice acceptor linked to the upstream PPP1R12C and a constitutive CAG promoter driven td-Tomato red fluorescent cistron flanked by a LoxP and an FRT site were stable integrated. Subsequently, upon stable selection for these reporters, these cells were lipofected with a plasmid expressing FlpO and Cre and the donor plasmid containing a LoxP and FRT flanked selection/reporter cassette and a dox-inducible mpClover/Luc2p/GDNF cistron-containing plasmid.

FIGS. 5A-5D shows that iNPCs are similar in composition to CNS10-NPCs. CNS10-NPCs and iNPCs were dissociated into single cells straight from cryopreservation and either process for single-cell RNAseq or plated and grown for 7 days in culture media. (FIG. 5A) Unbiased clustering of cells show iPSCs are separate from iNPCs (pink) and CNS10-NPCs. Some iNPCs are observed clustered with CNS10-NPCs. (FIG. 5B) Transcript expression of each cluster were scored with similarity to modules built of 25-50 genes per category. Scale: yellow=high expression, purple=low expression. (FIG. 5C) Feature plot of unbiased clusters show expression of mature and immature astrocyte-related transcripts in the iNPC and CNS10-NPC, but not iPSCs. (FIG. 5D) Immunocytochemistry on day 7 cultures of iNPC and CNS10-NPC show expression of S100p (gray) and GFAP (white) protein in both cell types in vitro. iNPC insert shows GFAP (gray) expression after 35 days cultured with astrocyte factors. Scale bar=75 μm, insert 25 μm.

FIGS. 6A-6D show constitutive and inducible constructs for engineering GDNF-expression in an iPSC line. (FIG. 6A) Illustrations showing components of AAVS1 targeted GDNF expression constructs. (FIG. 6B) GDNF ELISA demonstrates that iNPCs harboring the constitutive GDNF construct produce similar levels of GDNF to both lentivirally transduced iNPCs and CNS10-NPC cells. (FIG. 6C) GDNF ELISA of iNPCs with V1 inducible construct shows robust and efficient induction and attenuation in response to doxycycline addition and withdrawal. (FIG. 6D) iNPCs with V1 inducible construct fail to express GDNF when transplanted in the lumbar spinal cord of WT rats given doxycycline in drinking water. Scale bar=75 μm.

FIG. 7 shows TALEN targeting of AAVS1. Schematic showing Right and Left TALE nucleases as well as AAVS1 homology.

FIGS. 8A-8B show that iPSC-derived cells survive and protect ChAT+ motor neurons. SOD1G93A rats transplanted with current iNPCs transduced with lentivirus to produce GDNF in the lumbar spinal cord. (FIG. 8A) 10K cells/site n=5 animals. Transplants preserved host ChAT+ motor neurons. (FIG. 8B) 50K cells per site n=3 animals. Cells were not protective and displaced host neurons. Scale bar 250 μm.

FIG. 9A-9B shows engraftment of iNPCs in spinal cord of nude rats after 9 months. Lumbar spinal cords were sectioned and stained for human nuclei (SC121, green) and proliferative cells (Ki67, white). On left, lw magnification image of iNPC-GDNFCONST-V1 engraftment in one side of medial, ventral horn of spinal cord. Boxes denote higher magnification areas showing positive human cells (FIG. 9A) and negative human cells (FIG. 9B). Asterisk denotes central canal. Scale bar 100 μm and 25 μm in higher magnification.

FIG. 10 demonstrates a vector map of AAVS1-Tet-On-3G-GDNF (SEQ ID NO: 5).

FIG. 11 demonstrates a schematic of an exemplary differentiation protocol and timeline.

### DETAILED DESCRIPTION OF THE INVENTION

As described, the Inventors have pursued clinical studies as fetal neuroprogenitor cells (fNPCs) that are can differentiate into astrocytes and that can be transfected with lentivirus to stably produce GDNF. These GDNF-producing cells engraft efficiently in the spinal cord and slow the loss of ChAT+ motor neurons in the SOD1 ALS rat. Despite these breathtaking advances, fetal neuroprogenitor cells have several disadvantages. First, the availability and variation of fetal tissue as a non-renewable source. This includes the Inventors' clinical trials as based on a single line of fetal tissue, G010. There is a limited amount of material remaining of this line and no source to obtain new stock. The second major disadvantage of a fetal source of stem cells is random integration created using a lentiviral approach which generates a heterologous population when using a fetal neuroprogenitor cell source.

These limitations are specifically addressed by the neural progenitor cells derived from human induced pluripotent stem cells (iPSCs) provided herein. Addressing the first limitation of fetal cell sources, iPSCs are a renewable cell source capable of production on demand. The second limitation is obviated by clonal expansion capability of iPSCs, where one can uniformly introduce genetic constructs, including inducible expression systems or deploying genetic editing techniques such as CRISPR. Further advantages exist such as an unprecedented opportunity for autologous therapy, conceivably circumventing the complexities surrounding immunological rejection with allogeneic human cell transplantation. While iPSCs, including the Inventors' own work, have been used to produce neuronal progenitor-like cells, the iPSC-derived neural progenitor cells described herein are capable of engraftment in transplant host. This important property support feasibility of using the described cells for regenerative medicine via transplant.

Transplantation of human neural progenitor cells into the brain or spinal cord to replace lost cells, modulate the injury environment or create a permissive milieu to protect and regenerate host neurons has long been a promising therapeutic strategy for neurological diseases. Previously, the Inventors were able to transform adherent iPSCs into free-floating spheres (EZ spheres) capable of expansion. These EZ spheres could be differentiated towards NPC spheres with a spinal cord phenotype using a combination of all-trans retinoic acid (ATRA) and epidermal growth factor (EGF) and fibroblast growth factor-2 (FGF-2) mitogens. However, such cells did not engraft efficiently, thereby limiting their effective use in regenerative medicine therapies.

Advancing development of iPSC-derived NPCs towards clinical use can include: 1) safety; 2) maintenance of a normal cytogenetic status; 3) a lack of residual pluripotent cells to avoid possible malignant tumor formation; 4) reproducibility to expand the cells in large numbers; and 5) survival, integration, and engraftment of the cells provided herein into relevant central nervous system regions.

While neuronal replacement is one strategy for future clinical transplantation trials, astroglial cells are the most abundant cell type in the human brain and spinal cord and are now understood to be as important as neurons for brain function. They have also been implicated in a number of neurodegenerative diseases, with perhaps the best example being ALS. In ALS, glial dysfunction has been shown to lead to non-cell autonomous death of the motor neurons. Replacement of astrocytes, either naïve or secreting growth factors, has been shown to be beneficial in ALS models. The Inventors prior studies demonstrate that fNPCs can give rise to astroglial progenitors that then differentiate to immature and mature astrocytes within the rodent brain and spinal cord over long time periods. Human PSCs can also be directed into more mature astrocytes. While such PSC-derived mature astrocytes may survive transplantation, immature NPCs generated from iPSCs may provide cells that are easier to culture and expand in vitro and better suited to migrate, integrate and restore function in vivo.

Additionally, use of trophic factors to the brain using stem cell-derived neural progenitors is a powerful way to bypass the blood brain barrier. The delivery of various growth factors to the site of damage using ex vivo genetically modified cells has been shown to support host neurons in disease models of amyotrophic lateral sclerosis (ALS) and Parkinson's, Huntington's, and Alzheimer's Diseases. In parallel, delivery of glial cell line-derived neurotrophic factor (GDNF) has provided benefits to Parkinsonian patients and is currently being tested in a Phase 1/2a clinical trial for ALS patients. To fully exploit the benefits of trophic factors and ward off potential unwanted effects of trophic factors delivered by cells, regulation of growth factor secretion is a promising avenue for several neurodegenerative diseases. Chronic trophic factor delivery prohibits dose adjustment or shut off in the event of side effects as gene expression and downstream signaling activation are tightly connected processes. Lack of control over the timing and magnitude of gene expression could limit the efficacy of therapy and introduce unintended cellular effects.

Toward these ends, tetracycline (Tet)-regulated systems have been used to temporally and spatially regulate gene expression in various methodologies. This includes bacterial Tet transactivator (tTA) to silence gene expression downstream of a Tet-regulated promoter in the presence of doxycycline (dox), a Tet analog. In addition to this “Tet-Off” system, a “Tet-On” system uses a reverse tTA (rtTA) in order to activate transgene expression in the presence of dox The use of tTA and rtTA variants in neural stem cell populations is unexplored.

Here, the Inventors report a new protocol for the production of expandable human iPSC-derived neural progenitor cells (iPSC-derived NPCs). These human iPSC-derived NPCs could be easily propagated over long-term as suspension cultures and are similar to human fetal derived neural progenitor cells (fNPCs) that are demonstrated as safe and effective in clinical studies. Following injection, iNPCs successfully engraft with no signs of tumor formation or overgrowth, and again appear to perform similarly, if not superior, to analogous fNPC transplants. The Inventors' results describe a new source of human neural progenitor cells that do not have the supply, expansion and ethical concerns of fNPCs, and hence could be ideal for stem cell-based therapeutic approaches for neurodegenerative diseases such as ALS.

Based on the versatility of the generated iPSC-derived NPCs, these cells can serve as a wholly suitable and superior replacement for the successful fetal G010 cells in the context of ALS. Further development will involve evaluation of iPSC-derived NPCs growth and engraftment in the SOD-1 rat, and examination of long term tumorgenicity of these iPSC derived NPCs. This further includes additional efficacy studies in the SOD-1 rats. Further these studies will confirm the preliminary results observing SOD-1 rats showed engraftment in the spinal cord and a neuroprotective effect at certain cell doses in SOD-1. Additionally, tumorgenicity studies are in progress in nude rats as a safety/tumorgenicity study over 9 months.

Additional information is found, e.g., in U.S. App. Nos. 62/644,332, 62/773,752, PCT App. No. PCT/US2019/022595, PCT Pub. No. WO 2017/131926, Sareen et al. “Human neural progenitor cells generated from induced pluripotent stem cells can survive, migrate, and integrate in the rodent spinal cord” J. Comp. Neurol. 2014 Aug. 15; 522(12): 2707-2728, and Akhtar et al. “A Transposon-Mediated System for Flexible Control of Transgene Expression in Stem and Progenitor-Derived Lineages” Stem Cell Reports. 2015 Mar. 10; 4(3): 323-331, which are fully incorporated by reference herein.

Described herein is a method to generate induced pluripotent stem cell (iPSC)-derived neuronal progenitor cells (iNPCs). In various embodiments, the iNPCs provided herein are generated from a plurality of induced-pluripotent stem cells. In various embodiments, the iNPCs provided herein are generated from a plurality of cells that express at least one stem cell marker. The characteristics of iPSCs are discussed further below.

### Induced Pluripotent Stem Cells

Induced pluripotent cells are generated by reprogramming of differentiated somatic cells. Although differentiation is generally irreversible under physiological contexts, several methods have been developed to reprogram somatic cells to induced pluripotent stem cells (iPSCs). Exemplary methods are known to those of skill in the art and are described briefly herein below. The iPSCs provided herein can be generated by the methods described further below or they can be obtained from commercial sources, e.g., those available from ThermoFisher Scientific®, STEMCELL TECHNOLOGIES®, or Applied StemCell®.

Methods of reprogramming somatic cells into iPS cells are described, for example, in U.S. Pat. No. 8,129,187 B2; 8,058,065 B2; US Patent Application 2012/0021519 A1; Singh el al. Front. Cell Dev. Biol. (February, 2015); and Park el al., Nature 451: 141-146 (2008); which are incorporated herein by reference in their entireties. Specifically, iPSCs are generated from somatic cells by introducing a combination of reprogramming transcription factors. The reprogramming factors can be introduced as, for example, proteins, nucleic acids (mRNA molecules, DNA constructs or vectors encoding them) or any combination thereof. Small molecules can also augment or supplement introduced transcription factors. While additional factors have been determined to affect, for example, the efficiency of reprogramming, a standard set of four reprogramming factors sufficient in combination to reprogram somatic cells to an induced pluripotent state includes Oct4 (Octamer binding transcription factor-4), SOX2 (Sex determining region Y)-box 2, Klf4 (Kruppel Like Factor-4), and c-Myc. Additional protein or nucleic acid factors (or constructs encoding them) including, but not limited to LIN28+Nanog, Esrrb, Pax5 shRNA, C/EBPp, p53 siRNA, UTF1, DNMT shRNA, Wnt3a, SV40 LT(T), hTERT) or small molecule chemical agents including, but not limited to BIX-01294, BayK8644, RG108, AZA, dexamethasone, VPA, TSA, SAHA, PD0325901+CHIR99021(2i) and A-83-01 have been found to replace one or the other reprogramming factors from the basal or standard set of four reprogramming factors, or to enhance the efficiency of reprogramming.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells to be reprogrammed can be terminally differentiated somatic cells, as well as adult or somatic stem cells.

In some embodiments, reprogramming encompasses complete reversion of the differentiation state of a differentiated cell (e.g., a somatic cell) to a pluripotent state or a multipotent state. Reprogramming can result in expression of particular genes by the cells, the expression of which further contributes to reprogramming.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Isolated iPSC clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecat1, Esg1, Eras, Gdf3, Fgf4, Cripto, Dax1, Zpf296, Slc2a3, Rex1, Utf1, and Nat1, among others. In some embodiments, a cell that expresses Nanog and SSEA4 is identified as pluripotent.

In some embodiments of any of the aspects described herein, the cell described herein expresses at least one pluripotent stem cell marker. Methods for detecting the expression of stem cell markers can include, for example, RT-PCR and immunological methods that detect the presence of the encoded polypeptides, such as Western blots, immunocytochemistry or flow cytometric analyses. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of isolated clones. The cells are introduced to nude mice and histology and/or immunohistochemistry using antibodies specific for markers of the different germ line lineages is performed on a tumor arising from the cells. The growth of a tumor comprising cells from all three germ layers, endoderm, mesoderm and ectoderm further indicates or confirms that the cells are pluripotent stem cells.

Generally, throughout the differentiation process, a pluripotent cell will follow a developmental pathway along a particular developmental lineage, e.g., the primary germ layers—ectoderm, mesoderm, or endoderm.

The embryonic germ layers are the source from which all tissues and organs derive. The germ layers can be identified by the expression of specific biomarkers and gene expression. Assays to detect these biomarkers include, e.g., RT-PCR, immunohistochemistry, and Western blotting. Non-limiting examples of biomarkers expressed by early mesodermal cells include HAND1, ESM1, HAND2, HOPX, BMP10, FCN3, KDR, PDGFR-α, CD34, Tbx-6, Snail-1, Mesp-1, and GSC, among others. Biomarkers expressed by early ectoderm cells include but are not limited to TRPM8, POU4F1, OLFM3, WNT1, LMX1A and CDH9, among others. Biomarkers expressed by early endoderm cells include but are not limited to LEFTY1, EOMES, NODAL and FOXA2, among others. One of skill in the art can determine which lineage markers to monitor while performing a differentiation protocol based on the cell type and the germ layer from which that cell is derived in development.

### Methods

Induction of a particular developmental lineage in vitro is accomplished by culturing stem cells (e.g., the iPSCs provided herein) in the presence of specific agents, vectors, or combinations thereof that promote lineage commitment. Generally, the methods provided herein comprise the step-wise addition of agents (e.g., small molecules, growth factors, cytokines, polypeptides, vectors, etc.) into the cell culture medium or contacting a cell with agents that promote differentiation of the iPSCs to a neural progenitor cell lineage. In particular, transcription factors and growth factor signaling can be used to induce differentiation, which includes but is not limited to VegT, Wnt signaling (e.g., via β-catenin), bone morphogenic protein (BMP) pathways, fibroblast growth factor (FGF) pathways, and TGFβ signaling (e.g., activin A). See e.g., Sareen et al. J Comp Neurol. (2014), Baharvand H, et al. Neural differentiation from human embryonic stem cells in a defined adherent culture condition. Int J Dev Biol. 2007; 51:371-378, and which are incorporated herein by reference in their entireties.

In the context of cell ontogeny, the term “differentiate”, or “differentiating” is a relative term meaning a “differentiated cell” is a cell that has progressed further down the developmental pathway than its precursor cell. Thus, in some embodiments, a reprogrammed cell can differentiate to lineage-restricted precursor cells (such as a mesodermal stem cell), which in turn can differentiate into other types of precursor cells further down the pathway (such as a tissue specific precursor), and then to an end-stage differentiated cell, which plays a characteristic role in a certain tissue type, and may or may not retain the capacity to proliferate further.

Generally, in vitro-differentiated cells will exhibit a down-regulation of pluripotency markers (e.g., HNF4-α, AFP, GATA-4, and GATA-6) throughout the step-wise process and exhibit an increase in expression of lineage-specific biomarkers (e.g., mesodermal, ectodermal, or endodermal markers). See for example, Tsankov et al. Nature Biotech (2015), which describes the characterization of human pluripotent stem cell lines and differentiation along a particular lineage. The differentiation process can be monitored for efficiency by a number of methods known in the art. This includes detecting the presence of germ layer biomarkers using standard techniques, e.g., immunocytochemistry, RT-PCR, flow cytometry, functional assays, microscopy, etc.

### Methods:

The methods provided herein comprise the steps of (i) providing a quantity of induced pluripotent stem cells (iPSCs); (ii) culturing the iPSCs in the presence of a RHO kinase inhibitor; (iii) generating a monolayer; (iv) further culturing said iPSCs in the presence of LDN and SB; and additionally culturing said iPSCs in the presence of fibroblast growth factor (FGF), epidermal growth factor (EGF), and leukemia inhibitory factor (LIF), thereby generating iPSC-derived NPCs.

In various embodiments, the quantity of iPSCs provided herein includes iPSCs in suspension. In various embodiments, generating the monolayer includes shaking the cultured iPSCs.

In various embodiments, the iPSCs provided herein are cultured in the presence of a Rho kinase inhibitor (ROCK inhibitor). There are several Rho kinase inhibitors compatible with the current methods. In certain embodiments the Rho kinase inhibitor includes Fasudil, Ripasudil, Netarsudil, RKI-1447, Y-27632, GSK429286A, Y-30141, or any combination thereof. In certain embodiments, the Rho kinase inhibitor includes Y27632 dichloride hydrate. In certain embodiments, the iPSCs provided herein are cultured in a concentration of Rho kinase inhibitor of at least about 0.5 μM to about 12 μM. In certain embodiments, the iPSCs provided herein are cultured with a concentration of Rho kinase inhibitor of about 5 μM. In certain embodiments, the concentration of Rho kinase inhibitor is at least 1 μM or more, at least 2 μM or more, at least 3 μM or more, at least 4 μM or more, at least 5 μM or more, at least 6 μM or more, at least 7 μM or more, at least 8 μM or more, at least 9 μM or more, at least 10 μM or more, at least 11 μM or more, up to 12 μM. In certain embodiments, the Rho Kinase inhibitor is applied before plating of the cells and treatment with a media comprising one or more of LDN and/or SB.

In some embodiments, the iPSCs provided herein are cultured in the presence of a Rho kinase inhibitor (ROCK inhibitor for at least 8 hours or more, at least 12 hours or more, at least 24 hours or more, at least 48 hours or more, at least 36 hours or more, at least 72 hours or more, up to 3 days in culture.

In various embodiments, the iPSCs provided herein are cultured in the presence of one or more of LDN and SB.

LDN is a small molecule inhibitor of the activin receptor-like kinase (ALK) polypeptides. Specifically, LDN targets activin receptor-like kinase-2 (ALK2), ALK3 and ALK6 receptors. SB is a selective and potent inhibitor of the TGF-β/Activin/NODAL pathway that inhibits ALK5 (IC₅₀=94 nM), ALK4 (IC₅₀=140 nM), and ALK7 by competing for the ATP binding site. It does not inhibit the BMP type I receptors ALK2, ALK3, and ALK6 (see, e.g., Inman et al. (2002) Molecular pharmacology 62 1 65-74.; and Tchieu et al. Cell Stem Cell 21(3) 399-410.e7. (2017); and Laping et al. (2002) Molecular pharmacology. 62 1 58-64, which are incorporated herein by reference in their entireties). Inhibition of ALK promotes differentiation of progenitor cells and iPSCs by inhibiting the bone morphogenetic (BMP) pathway and TGFβ signaling pathways. The inhibitory cocktail of LDN (e.g., LDN193189) in combination with SB (e.g., SB431542) allows for the efficient generation of central nervous system cells by dual SMAD inhibition (dSMADi). Modifications of dSMADi can yield many different neural subtypes along the neuroaxis of the embryo including forebrain, midbrain and spinal cord progenitor cells (see e.g., Tchieu et al. Cell Stem Cell 21(3) 399-410.e7. (2017)). There are several LDN small molecules, SB small molecules, and ALK inhibitors known in the art, including but are not limited to, LDN193189 (e.g., STEMCELL TECHNOLOGIES-Catalog #72147), SB-431542 (GlaxoSmithKline®), LDN189, LDN-212854, and derivatives thereof.

In other embodiments, the method provided herein further comprises culturing the iPSCs in the presence of LDN and SB for about 7 days up to about 13 days in culture. In other embodiments, the method comprises further culturing the iPSCs in the presence of LDN for at least 48 hours or more, at least 36 hours or more, at least 72 hours or more, at least 3 days or more, at least 4 days or more, at least 5 days or more, at least 6 days or more, at least 7 days or more, at least 8 days or more, at least 9 days or more, at least 10 days or more, at least 11 days or more, at least 12 days or more, at least 13 days or more, at least 14 days or more, up to about 15 days in culture. In other embodiments, the method comprises further culturing the iPSCs in the presence of SB for at least 48 hours or more, at least 36 hours or more, at least 72 hours or more, at least 3 days or more, at least 4 days or more, at least 5 days or more, at least 6 days or more, at least 7 days or more, at least 8 days or more, at least 9 days or more, at least 10 days or more, at least 11 days or more, at least 12 days or more, at least 13 days or more, at least 14 days or more, up to about 15 days in culture. In other embodiments, the method comprises further culturing the iPSCs in the presence of LDN and SB for at least 48 hours or more, at least 36 hours or more, at least 72 hours or more, at least 3 days or more, at least 4 days or more, at least 5 days or more, at least 6 days or more, at least 7 days or more, at least 8 days or more, at least 9 days or more, at least 10 days or more, at least 11 days or more, at least 12 days or more, at least 13 days or more, at least 14 days or more, up to about 15 days in culture.

In other embodiments, the method comprises culturing the iPSCs provided herein in the presence of at about 4.75 to about 5.75 μg/mL. In other embodiments, the method comprises culturing the iPSCs provided herein in the presence of a concentration of at least 3.0 μg/mL or more LDN, at least 3.25 μg/mL or more LDN, at least 4.25 μg/mL or more LDN, at least 4.5 μg/mL or more LDN, at least 4.75 μg/mL or more LDN, at least 5.0 μg/mL or more LDN, at least 5.25 μg/mL or more LDN, at least 5.5 μg/mL or more LDN, at least 5.75 μg/mL or more LDN, at least 6.0 μg/mL or more LDN, at least 6.25 μg/mL or more LDN, at least 6.5 μg/mL or more LDN, at least 6.75 μg/mL or more LDN, at least 7.0 μg/mL or more LDN, at least 7.25 μg/mL or more LDN, at least 7.5 μg/mL or more LDN, at least 7.75 μg/mL or more LDN, up to 8 μg/mL LDN.

In other embodiments, the method comprises culturing the iPSCs provided herein in the presence of at about 0.5 μM to about 4 μM SB. In other embodiments, the method comprises culturing the iPSCs provided herein in the presence of a concentration of at least 0.5 μM or more SB, at least 0.75 μM or more SB, at least 1.0 μM or more SB, at least 1.25 μM or more SB, at least 1.5 μM or more SB, at least 1.75 μM or more SB, at least 2.0 μM or more SB, at least 2.25 μM or more SB, at least 2.5 μM or more SB, at least 2.75 μM or more SB, at least 3.0 μM or more SB, at least 3.25 μM or more SB, at least 3.5 μM or more SB, at least 3.75 μM or more SB, up to 4.0 μM SB.

In other embodiments, the iPSCs provided herein are further cultured in the presence of one or more of fibroblast growth factor (FGF), epidermal growth factor (EGF), and leukemia inhibitory factor (LIF) for at least 8 hours or more, at least 12 hours or more, at least 24 hours or more, at least 48 hours or more, at least 36 hours or more, at least 72 hours or more, at least 3 days or more, at least 4 days or more, at least 5 days or more, at least 6 days or more, at least 7 days or more, at least 8 days or more, at least 9 days or more, at least 10 days or more, at least 11 days or more, at least 12 days or more, at least 13 days or more, at least 14 days or more, at least 15 days or more, up to 16 days in culture.

In other embodiments, the iPSCs provided herein are further cultured in the presence of fibroblast growth factor (FGF), epidermal growth factor (EGF), and leukemia inhibitory factor (LIF) for at least 8 hours or more, at least 12 hours or more, at least 24 hours or more, at least 48 hours or more, at least 36 hours or more, at least 72 hours or more, at least 3 days or more, at least 4 days or more, at least 5 days or more, at least 6 days or more, at least 7 days or more, at least 8 days or more, at least 9 days or more, at least 10 days or more, at least 11 days or more, at least 12 days or more, at least 13 days or more, at least 14 days or more, at least 15 days or more, up to 16 days in culture.

In other embodiments, the iPSCs provided herein are further cultured in the presence of fibroblast growth factor (FGF) at a concentration of at least about 50 ng/ml up to about 200 ng/ml. In other embodiments, the iPSCs provided herein are further cultured in the presence of fibroblast growth factor (FGF) at a concentration of at least 50 ng/ml or more, at least 75 ng/ml or more, at least 100 ng/ml or more, at least 125 ng/ml or more, at least 150 ng/ml or more, at least 175 ng/ml or more to about 200 ng/ml.

In other embodiments, the iPSCs provided herein are further cultured in the presence of epidermal growth factor (EGF) at a concentration of at least about 50 ng/ml up to about 200 ng/ml. In other embodiments, the iPSCs provided herein are further cultured in the presence of EGF at a concentration of at least 50 ng/ml or more, at least 75 ng/ml or more, at least 100 ng/ml or more, at least 125 ng/ml or more, at least 150 ng/ml or more, at least 175 ng/ml or more to about 200 ng/ml.

In other embodiments, the iPSCs provided herein are further cultured in the presence of leukemia inhibitory factor (LIF) at a concentration of at least about 50 ng/ml up to about 200 ng/ml. In other embodiments, the iPSCs provided herein are further cultured in the presence of LIF at a concentration of at least 50 ng/ml or more, at least 75 ng/ml or more, at least 100 ng/ml or more, at least 125 ng/ml or more, at least 150 ng/ml or more, at least 175 ng/ml or more to about 200 ng/ml.

The iPSCs can be cultured in the presence of any one or more of the factors described above. In other embodiments, the iPSCs are cultured in the presence of each of the following factors: a RHO kinase inhibitor; LDN; SB; FGF; EGF; and LIF in combination.

In some embodiments of any one of the aspects, the method provided herein comprises a step of iNPC differentiation. An exemplary iNPC differentiation protocol is as follows:
1. Start with any # of wells of iPSC grown in 6 well plates at about 80-100% confluency. If cells are at a higher density and plate well, it is acceptable to do a larger split. For example: 2 wells->seed 3 wells. Generally, it is safest to split at a 1:1 ratio. A fully confluent well (no gaps) is necessary post-split so any split ratio that achieves this is acceptable.
2. Aspirate media and add 1.5 mL of Accutase™ to each well.
3. Incubate at 37° C., 5% C02 for approximately 5 minutes (+2-5 minutes if needed).
a. After 3 minutes in the incubator, tap the sides of the plate gently to facilitate lifting of the cells.
4. Once cells have detached, add 1.5 mL of mTesR™ media to each well and continue to gently wash cells off the bottom of the well. If cells remain stuck to the bottom of the well, gently scrape off cells using the pipet tip while slowly dispensing media.
5. Transfer cell suspension to a labeled 15 mL conical tube.
6. Spin down cell suspension for 3 min at 1,500 rpm (˜300 g)
7. Gently aspirate supernatant, taking care not to disturb cell pellet
8. Resuspend cell pellet gently in mTesR media+ROCK inhibitor at working concentration of 5 uM.
a. Volume to resuspend in is dependent on number of wells seeded
9. Seed 3 mLs per well of cells+mTeSR+ROCK inhibitor.
10. Transfer to incubator and gently shake plate in X-Y motion to evenly distribute cells.
11. The following day: if cells have sufficiently attached and formed a nice monolayer, proceed with differentiation protocol outlined below—full media changes, feeding 3 mL per day gently down the side of the well.
a. Day 1-10—full media change into Neural Induction Media (NIM)
b. After 10 feeds of NIM, change media on each well to SEFL containing 5 uM ROCK inhibitor. Then use a cell scraper to lift cells from each well and transfer into a T25 flask.
c. The following day—do a 50-75% media change with fresh SEFL media. Remove as much debris as possible. Larger media changes can be done in the first few days of sphere culture to remove debris as needed.
d. Maintain as spheres—feed 50% every 3^{rd} day if not conditioning faster.

### Sphere Culture and Maintenance:

Feed spheres every 3^{rd} day if not conditioning media beforehand
Media should generally be conditioned every other day, indicated by an orange-yellow media color.
Spheres are chopped every 7-10 days, based on size and density.
Should cell clumping and adherence to bottom of the flask occur, triturate spheres gently and wash media down the bottom of the flask to dislodge cells.

TABLE 1
Reagents for neural induction media

| Neural | | | | | | |
|---|---|---|---|---|---|---|
| Induction | | | | | Volume added | |
| Media | Stock Conc | | Working Conc | | per 100 mL | |
| DMEM/F12 | 1X | | 50% | | 50 | mL |
| Neurobasal | 1X | | 50% | | 50 | mL |
| B27+ | 50x | | 0.5X | | 1 | mL |
| N2 | 100x | | 1X | | 1 | mL |
| L-glut | 200 mM (100X) | | 1X (2 mM) | | 1 | mL |
| NEAA | 100x | | 1X | | 1 | mL |
| P/S | 100X | | 0.5X | | 500 | uL |
| Insulin | 9.5-11.5 | mg/ml | 4.75-5.75 | ug/mL | 50 | uL |
| LDN | 10 | mM | 0.4 | uM | 4 | uL |
| SB | 10 | mM | 2 | uM | 20 | uL |

TABLE 2
Reagents for cortical sphere growth media

| Cortical Sphere | | | | | | |
|---|---|---|---|---|---|---|
| Growth Media | Stock Conc | | Working Conc | | Ex. 100 mL | |
| Stemline NSC Expansion | 1X | | 1X | | 100 | mL |
| Medium | | | | | | |
| Heparin | 5 | mg/mL | 5 | ug/mL | 100 | uL |
| FGF | 100 | ug/mL | 100 | ng/mL | 100 | uL |
| EGF | 100 | ug/mL | 100 | ng/mL | 100 | uL |
| LIF | 10 | ug/mL | 10 | ng/mL | 100 | uL |

### Vectors

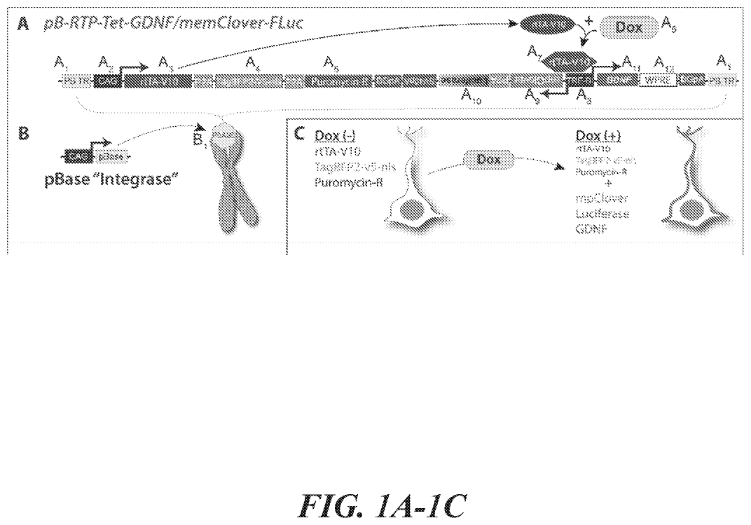

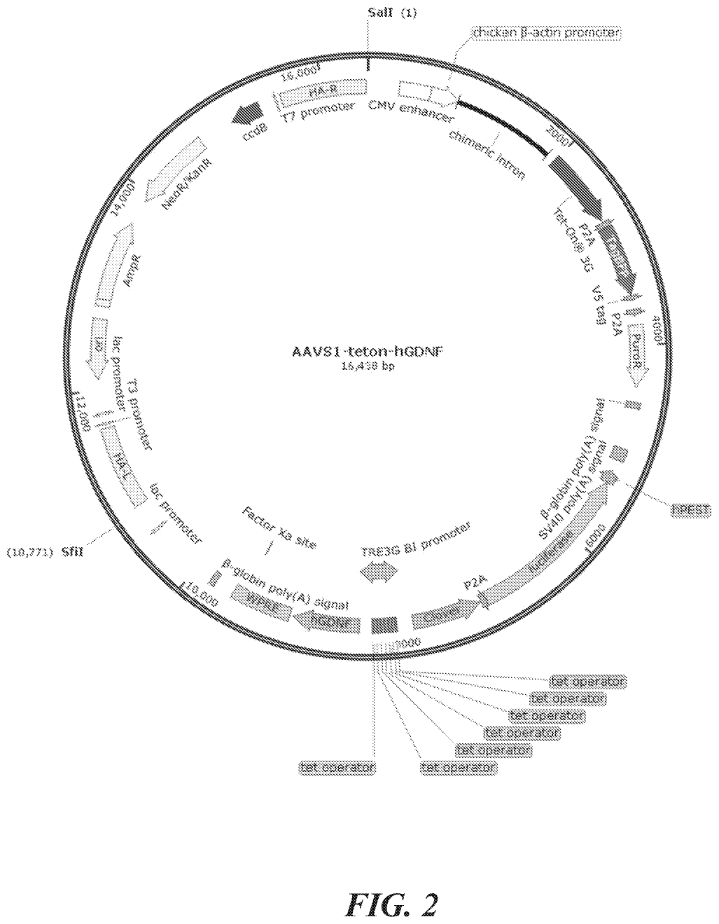

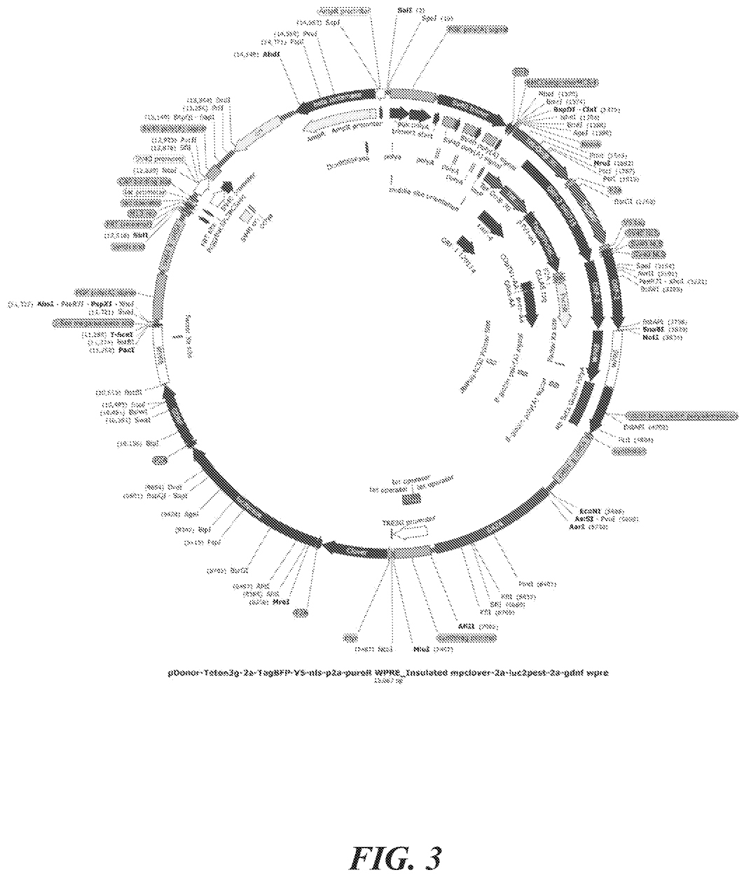

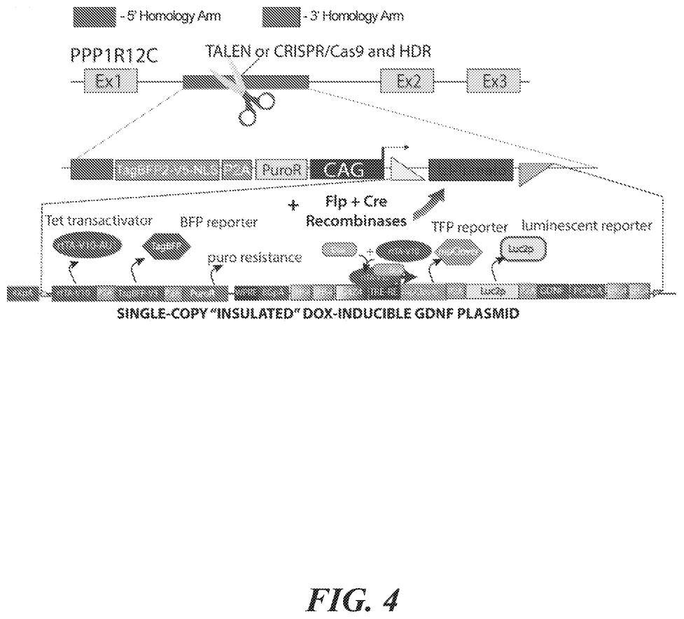

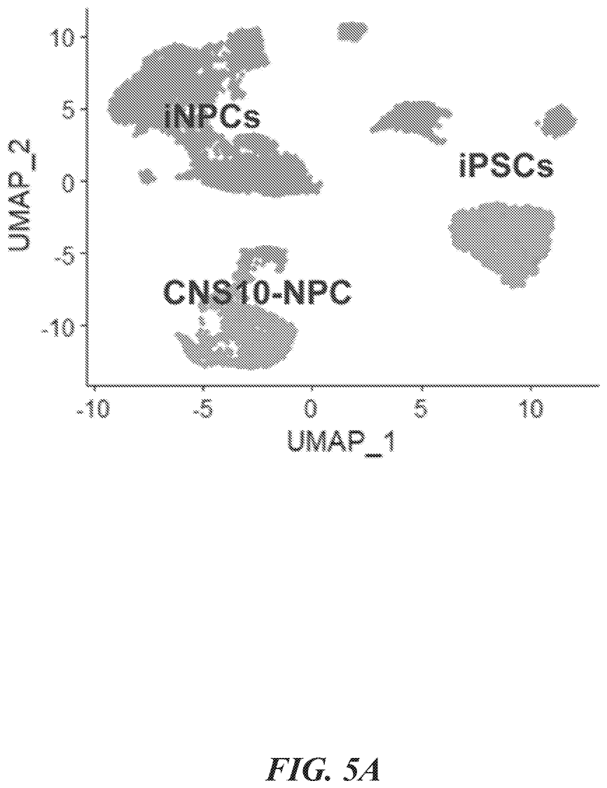

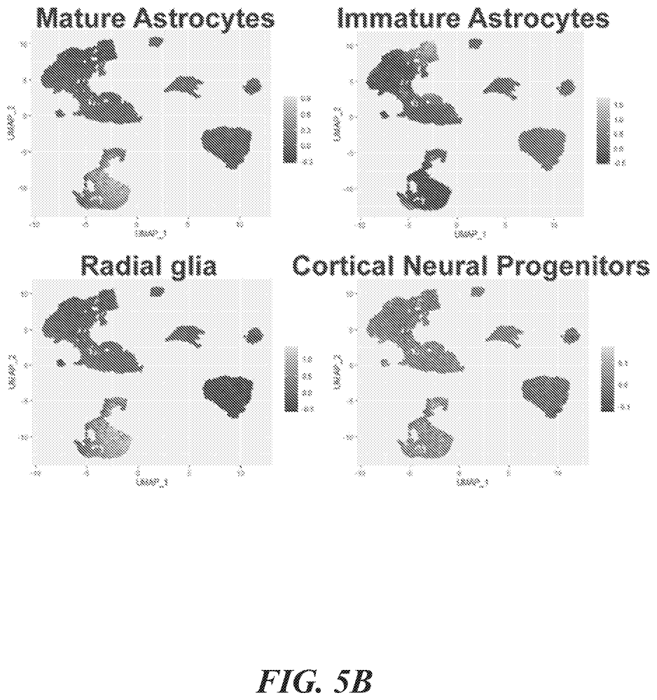

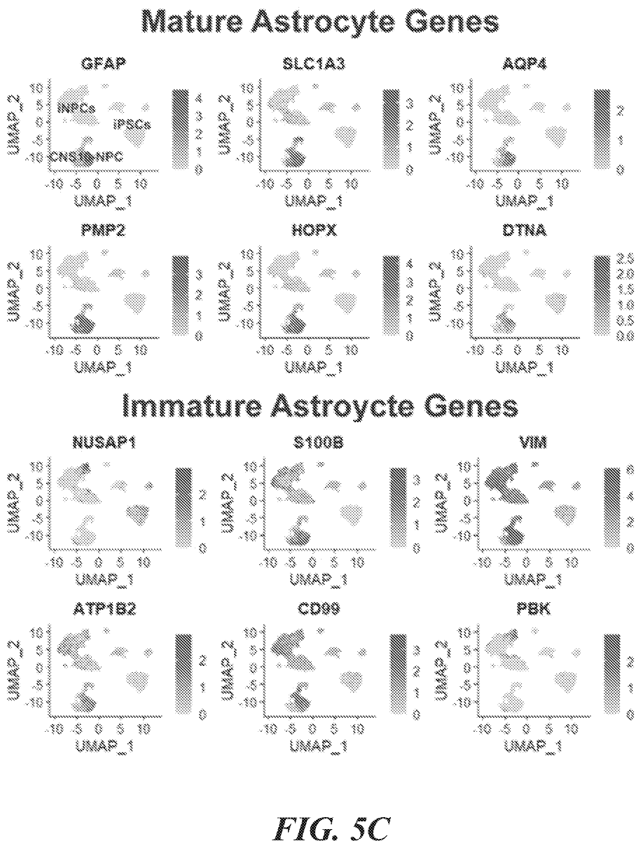

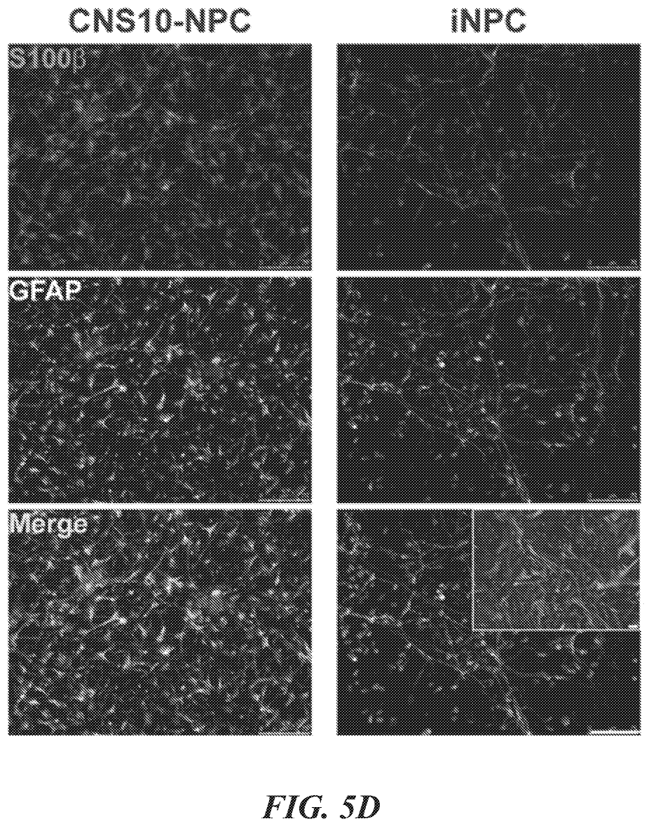

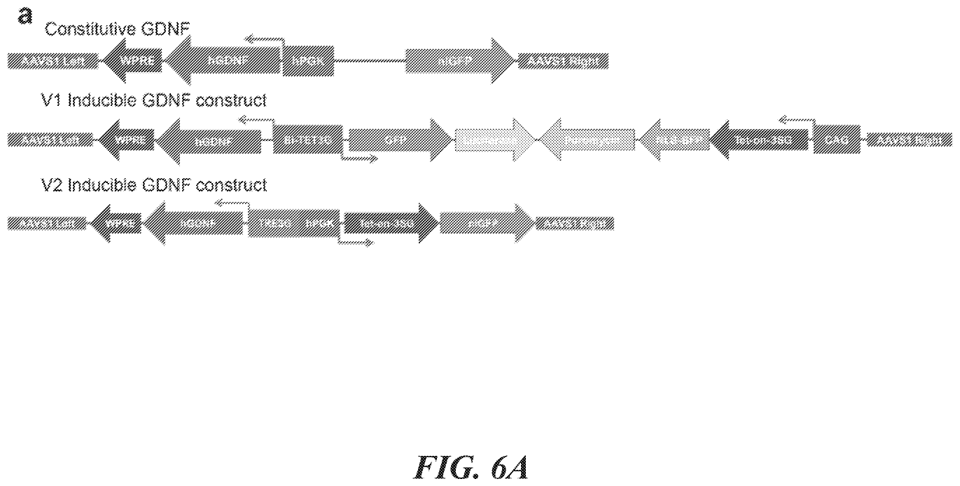

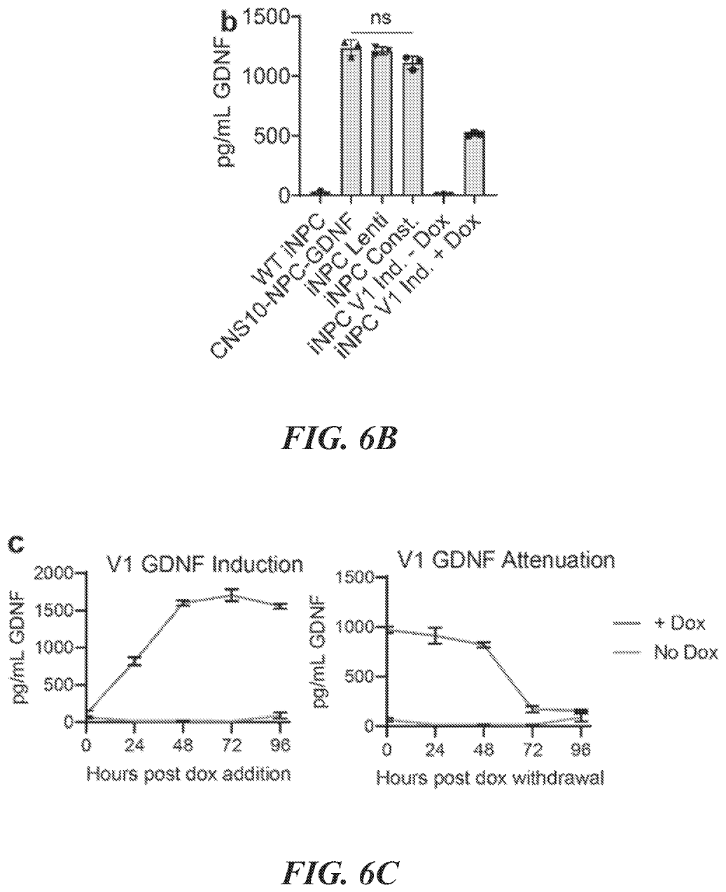

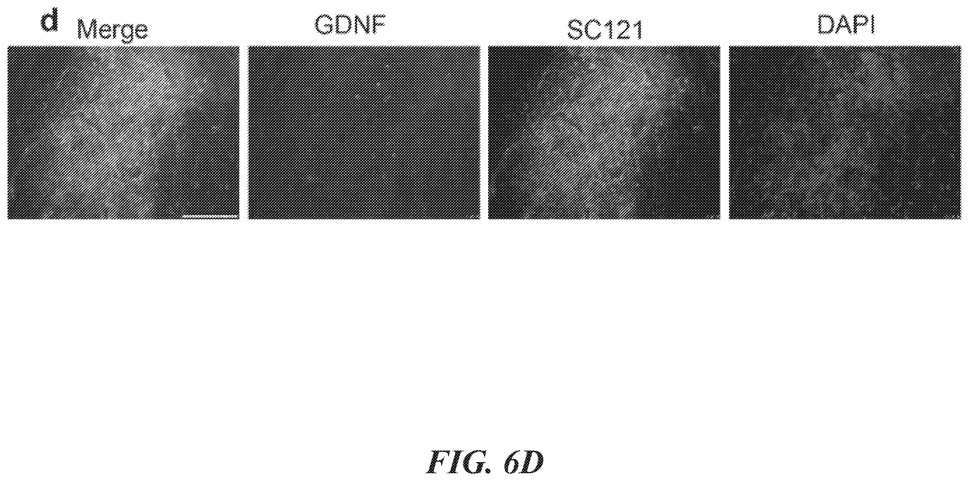

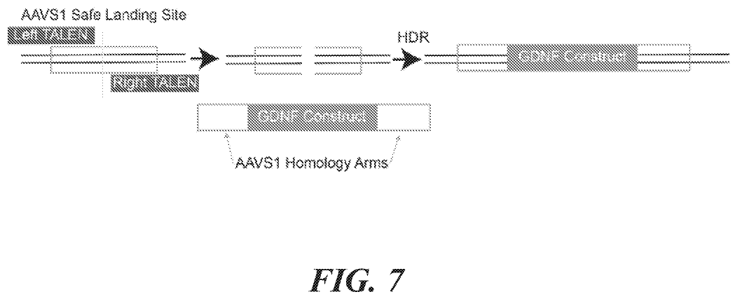

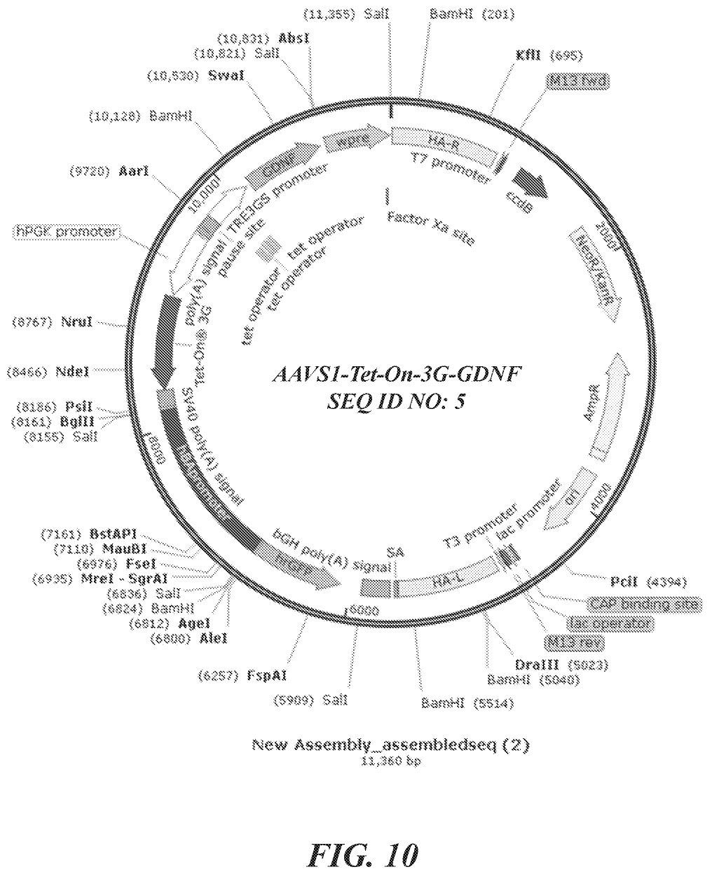

Described herein is a method, including providing a quantity of iPSC-derived NPCs made by the aforementioned methods of generating induced pluripotent stem cell (iPSC)-derived neuronal progenitor cells (NPCs), and introducing at least two vectors into the iPSC-derived NPCs. Described herein is a method, including providing a quantity of iPSC-derived NPCs made by the aforementioned method of generating induced pluripotent stem cell (iPSC)-derived neuronal progenitor cells (NPCs), and introducing at least one vector into the iPSC-derived NPCs. Exemplary vectors are described in FIG. 1A-FIG. 4, FIG. 6A, FIG. 7, and FIG. 10.

iPSC-derived NPCs produced by the methods described herein can be genetically engineered to express peptides and proteins, including therapeutic agents used for treating diseases such as ALS. Exemplary vectors are shown in FIG. 1A-FIG. 4, FIG. 6A, FIG. 7, and FIG. 10.

A vector is a nucleic acid construct designed for delivery to a host cell or for transfer of genetic material between different host cells. As used herein, a vector can be viral or non-viral. The term “vector” encompasses any genetic element that is capable of replication when associated with the proper control elements and that can transfer genetic material to cells. A vector can include, but is not limited to, a cloning vector, an expression vector, a plasmid, phage, transposon, cosmid, artificial chromosome, virus, virion, etc.

In some embodiments, the vector is selected from the group consisting of: a plasmid and a viral vector.

An expression vector is a vector that directs expression of an RNA or polypeptide from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in animal cells for expression and in a prokaryotic host for cloning and amplification. “Expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene.

In other embodiments, the vector or vectors provided herein comprise a piggyBac vector and a pBase vector. In other embodiments, the piggyBac vector comprises in a 5′ to 3′ direction: an expression cassette, including: a constitutive promoter, an inducible, bi-directional polycistronic promoter including a tet responsive element, and a sequence encoding a protein or peptide, two transposon elements, wherein the two transposon elements flank the expression cassette and at least one homologous recombination sequence.

In other embodiments, the sequence encoding the protein or peptide includes a neurotrophic factor. In other embodiments, neurotrophic factor includes glial derived neurotrophic factor (GDNF). The coding sequences for human GDNF is known in the art, e.g., NCBI Gene ID 2668, >NC_000005.10:c37840044-37812677 Homo sapiens chromosome 5, GRCh38.p13 Primary Assembly (SEQ ID NO: 2). The RNA transcript sequences for human GDNF are also known in the art, e.g., NCBI Reference Sequence: NM_000514.4 Homo sapiens glial cell derived neurotrophic factor (GDNF), transcript variant 1, mRNA (SEQ ID NO: 3).

Furthermore, the amino acid sequence of human GDNF and variants thereof are known in the art, e.g., glial cell line-derived neurotrophic factor isoform 1 preproprotein [Homo sapiens], NCBI Reference Sequence: NP_000505.1 (SEQ ID NO: 4).

In other embodiments, the piggyBac vector provided herein comprises in a 5′ to 3′ direction: an expression cassette, including: a constitutive promoter, an inducible, bi-directional polycistronic promoter including a tet responsive element, and a sequence encoding GDNF, two transposon elements, wherein the two transposon elements flank the expression cassette and at least one homologous recombination sequence.

In other embodiments, the tet responsive element drives in vivo expression of the GDNF transgene.

In other embodiments, homologous recombination sequence includes a sequence capable of targeting a genomic safe harbor. In other embodiments, genomic safe harbor is one of: the adeno-associated virus site 1 (AAVS1), the chemokine (C—C motif) receptor 5 (CCR5) gene, human orthologue of the mouse Rosa26 locus. In other embodiments, neuronal progenitor cells are engrafting neuronal progenitor cells.

An example of the aforementioned vector includes pB-RTP-Tet-GDNF/memClover-FLuc” [piggyBac-Reverse transactivator/TagBFP2nls/PacR-Tet inducible-GDNF/membrane Clover-Firefly Luciferase] [SEQ ID NO: 1], which is depicted in FIG. 1A.

Here, the vector includes two promoters—a constitutively active CMV/Chick β-Actin (aka CAG) promoter and an inducible, bi-directional TRE-Bi promoter. The CAG promoter drives constitutive expression of the rtTA-V10 (aka tet-ON) transactivator, TagBFP2-V5nls (enhanced blue fluorescent protein with a V5 tag and nuclear localization sequence), and the puromycin resistance gene. Transgenes in tandem are separated by self-cleaving peptide linkers (P2A). Here, addition of a tetracycline analog or derivative, doxycycline, causes the rtTA-V10 transactivator to bind to the TRE-Bi promoter and catalyze transcription of downstream transgenes. The first cistron of the TRE-Bi promoter harbors a myristoylated and palmitoylated (MyrPalm) clover reporter (mpClover) followed by destabilized firefly luciferase (Luc2P). The second cistron downstream of the inducible TRE-Bi promoter can encode a neurotrophic factor such as GDNF followed by the woodchuck hepatitis virus post-transcriptional element (WPRE) for increased gene expression Rabbit beta-globin polyAs were placed downstream of the respective elements to terminate transcription and prevent spurious transgene expression. The pB-RTP-Tet-GDNF memClover-FLuc vector can be transfected alongside a pBase plasmid to promote stable genomic integration. Another example of the aforementioned vector includes pDonor-Teton3 g-2a-TagBFP-V5-nls-p2a-puroR WPRE_Insulated mpclover-2a-luc2pest-2a-gdnf wpre.

In other embodiments, at least one vector includes an expression cassette, including, a constitutive or inducible promoter operably linked to a sequence encoding a protein or peptide, at least one homologous recombination sequence. In other embodiments, the protein or peptide includes a neurotrophic factor. In other embodiments, the neurotrophic factor includes glial derived neurotrophic factor (GDNF). In other embodiments, the constitutive promoter is 3-phosphoglycerate kinase (PGK promoter). In other embodiments, the homologous recombination sequence includes a sequence capable of targeting a genomic safe harbor. In other embodiments, the genomic safe harbor is one of: the adeno-associated virus site 1 (AAVS1), the chemokine (C—C motif) receptor 5 (CCR5) gene, human ortholog of the mouse Rosa26 locus. In other embodiments, the inducible promoter includes a promoter regulated by a tetracycline-class antibiotic. In other embodiments, the tetracycline-class antibiotic includes doxycycline. In other embodiments, the inducible promoter is regulated by a reverse tetracycline-controlled transactivator (rtTA) or a tet-On advanced transactivator (rtTA2S-M2). In other embodiments, the iPSC-derived NPCs are engrafting iPSC-derived NPCs. In other embodiments, the iPSC-derived NPCs express a genomically integrated expression cassette. In other embodiments, the genomically integrated expression cassette is at a genomic safe harbor.

Also described herein is a quantity of cells made by the aforementioned methods, wherein the iPSC-derived NPCs express a genomically integrated expression cassette. For example, a quantity of iPSC-derived NPCs capable of inducible expression of glial derived neurotrophic factor (GDNF) made by a method including providing a quantity iPSC-derived NPCs, and introducing at least one vector. In other embodiments, the at least one vector includes introducing a piggyBac vector and a pBase vector into the iPSC-derived NPCs, wherein the piggyBac vector includes a constitutive promoter, an inducible, bi-directional polycistronic promoter including a tet response element, and a sequence encoding GDNF. In various embodiments, the cells provided herein are substantially homogeneous.

In various embodiments, the vector includes at least one homologous recombination sequence. In other embodiments, the cells express an expression cassette from one or more vectors. In other embodiments, the cells expressing the expression cassette from the one or more vectors have been nucleofected, transfected, or electroporated or other gene delivery techniques known in the art. In other embodiments, the one or more vectors includes a piggyBac vector, a pBase vector, or both. In other embodiments, the piggyBac vector includes at least two promoters, wherein at least one promoter is inducible. In other embodiments, the least one inducible promoter is polycistronic. In other embodiments, the at least one inducible, polycistronic promoter is bi-directional. In other embodiments, the expression cassette is genomically integrated. In other embodiments, the expression cassette encodes the therapeutic protein or peptide. In other embodiments, the therapeutic protein or peptide includes a neurotrophic factor.

In various embodiments, the one or more vectors include a vector with a gene expression cassette flanked by two transposon elements. In various embodiments, the two transposon elements include piggyBac terminal repeats (PB TR). In various embodiments, the vector includes the constitutive promoter includes CMV/Chick β-Actin (aka CAG) promoter. In various embodiments, the vector includes an includible, bi-directional promoter includes TRE-Bi promoter. In various embodiments, the constitutive promoter is operatively linked to a tet response elements. In various embodiments, the “tet-on” element including for example, rTA. In other embodiments, rTA includes rtTA-V10. In various embodiments, the constitutive promoter is operatively linked to a selection factor, including for example neomycin or puromycin. In various embodiments, the inducible, bi-directional promoter is polycistronic. In various embodiments, the inducible bi-directional promoter is operatively linked to elements in a first, second or third or more cistrons. In various embodiments, a first, second, or third, or more cistrons includes a transgene. In various embodiments, the transgene is followed by one or more post-transcriptional elements. In various embodiments, the one or more post-transcriptional element includes woodchuck hepatitis virus post-transcriptional element (WPRE).

In various embodiments, the transgene is followed by one or more poly-A tails. In this includes, for example, rabbit beta-globin polyAs. In various embodiments, the transgene is a neurotrophic factor. In various embodiments, the neurotrophic factor includes glial derived neurotrophic factor (GDNF). In other embodiments, the one or more vectors include a vector encoding a recombinase including VCre (Vlox and derivatives), SCre (Slox and derivatives), Dre (Rox and derivatives), and phiC31 (attb) or other recombinases known in the art.

In various embodiments, the vector includes one or more elements promoting target of safe landing sites, including AAVS1. In some embodiments, the vector comprises a AAVS1 targeted GDNF expression construct. The AAVS1 site provided herein remains in a region of open chromatin, constructs inserted here are useful in retaining the expression in differentiated cell progeny (e.g., of the iNPCs).

In various elements, one or more insulator elements around the inducible cassette attenuates potential silencing during cell differentiation. In various embodiments, the expression cassette includes one or more sub-cassettes, wherein each sub-cassette includes 1) a promoter 2) a transgene and 3) a polyA transcription stop element. In various embodiments, the expression cassette including one or more sub-cassettes includes a constitutive sub-cassette and an inducible sub-cassette. For example, the constitutive sub-cassette includes the constitutive promoter expressing rTA transactivator, and the inducible sub-cassette includes an inducible promoter expressing a neurotrophic factor such as GDNF and optionally one or more reporter proteins.

In various embodiments, the vector includes at least one homologous recombination sequence. In other embodiments, the homologous recombination sequence includes a sequence capable of targeting a genomic safe harbor. In other embodiments, the genomic safe harbor is one of: the adeno-associated virus site 1 (AAVS1), the chemokine (C—C motif) receptor 5 (CCR5) gene, human ortholog of the mouse Rosa26 locus.

In some embodiments, a vector provided herein is capable of driving expression of one or more sequences in a cell using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant expression vector is capable of directing expression of the exogenous sequence preferentially in a particular cell type (e.g., tissue-specific regulatory elements are used to express the nucleic acid in, for example, an iPSC or a neural progenitor cell provided herein). Tissue-specific regulatory elements are known in the art. For example, neuron-specific promoters are discussed, in Byrne and Ruddle, 1989. Proc. Natl. Acad. Sci. USA 86: 5473-5477). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

Methods of generating the vectors provided herein are known in the art and discussed above. By way of example only, the polypeptide expression (e.g., neurotrophic factors, GDNF) can be regulatable. This can be achieved, e.g., by a TALEN and a homologous recombination approach (see, e.g., FIG. 7 and Akhtar et al. Inducible Expression of GDNF in Transplanted iPSC-Derived Neural Progenitor Cells. Stem Cell Reports. 2018; 10(6):1696-1704. doi:10.1016/j.stemcr.2018.03.024, which is incorporated herein by reference in its entirety).

In some embodiments, the nucleic acid sequence provided herein is delivered to the cell described herein via an integrating vector. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vectors.

In some embodiments, the nucleic acid sequence provided herein is delivered to the cell described herein via a non-integrative vector. Non-integrative vectors include non-integrative viral vectors. Non-integrative viral vectors eliminate one of the primary risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. Containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Other non-integrative viral vectors include adenoviral vectors and the adeno-associated viral (AAV) vectors.

Another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages). This permits a self-limiting transient expression of a chosen heterologous gene or genes in a target cell.

Another example of a non-integrative vector is a minicircle vector. Minicircle vectors are circularized vectors in which the plasmid backbone has been released leaving only the eukaryotic promoter and cDNA(s) that are to be expressed.

As noted above, in some embodiments, the nucleic acid sequences provided herein are expressed in the cells from a viral vector. A “viral vector” includes a nucleic acid vector construct that includes at least one element of viral origin and has the capacity to be packaged into a viral vector particle. The viral vector can contain a nucleic acid encoding a polypeptide described herein in place of non-essential viral genes. The vector and/or particle may be utilized for the purpose of transferring nucleic acids into cells either in vitro or in vivo.

Methods of introducing vectors into cells are known in the art, e.g., via nucleofection, transfection, electroporation, or other. In other embodiments, the method of introducing at least two vectors comprises one or more of: nucleofection, transfection and electroporation. In other embodiments, the introducing at least one vector includes one or more of: nucleofection, transfection and electroporation. However, the nucleic acid sequences and vectors provided herein can be delivered using any transfection reagent or other physical means that facilitates entry of nucleic acids into a cell.

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g., in vitro or ex vivo administration) or target cells (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Clones that express the desired construct and or vectors provided herein can be selected based on a reporter polypeptide expression (e.g., GFP or a fluorescent tag), and integration into the AAVS1 site can be confirmed through nucleic acid sequencing. The final vector can be modified to remove the reporter polypeptide sequence or tag by methods known in the art. In some embodiments, the vector excludes a reporter polypeptide sequence. In some embodiments, the iNPCs to be engrafted into a subject do not express a fluorescent tag.

iNPCs

The induced-pluripotent stem cells (iPSCs) provided herein that express the constructs provided herein can be selected for differentiation, expansion, and cell banking by methods known in the art. The iNPCs will be generated by culturing the iPSCs by the afformentioned methods described above and evaluated for iNPC markers. Non-limiting examples of neural progenitor cell markers include: Nestin, VIM, TUBB3, MAP2, APQ4, S100β, SC121, ChAT, BCL11B, SATB2, Annexin V, and GFAP. The iNPCs provided herein will also have the ability to establish and generate astrocytes. Non-limiting examples of markers for astrocytes include GFAP, EAAT1/GLAST, EAAT2/GLT-1, glutamine synthetase, S100β, and ALDH1L1.

In some embodiments, the iNPCs provided herein have at least one phenotypic characteristic of a neural progenitor cell. Characterization of iNPCs can be carried out by methods known in the art, e.g., qPCR for established NPC genes or markers, plate-downs, immunocytochemistry (ICC), Western blotting, microscopy, and functional assays (e.g., metabolic assays or electrophysiological assays). ICC and ELISAs for GDNF can be used to characterize the in vitro levels of GDNF production and dynamics of tetracycline regulation.

In various embodiments, the iPSC-derived NPCs are capable of serial passaging as a cell line.

In other embodiments, the iPSC-derived NPCs (iNPCs) are capable of aggregating. In other embodiments, the iNPCs are aggregated as a plurality of neurospheres. As used herein, the term “neurosphere” refers to an aggregate of a plurality of cells that express at least one neuronal cell marker. The iNPCs provided herein express markers of cortical neural progenitors as well as genes associated with both mature astrocytes and immature astrocytes. The iNPCs provided herein can expand as spheres or aggregates, develop filopodia in culture, express neural progenitor markers (e.g., nestin), and/or differentiate into astrocytes.

In other embodiments, the iPSC-derived NPCs are engrafted into a tissue in a subject. In other embodiments, the iNPC neurospheres are engrafted into a subject.

### Cell Compositions for Engraftment

The methods of administering human iNPCs to a subject as provided herein involve the use of therapeutic compositions comprising such cells. Therapeutic compositions contain a physiologically tolerable carrier together with the cell composition and optionally at least one additional bioactive agent, polypeptide(s), nucleic acid(s) encoding said polypeptide, or factor(s) as described herein, dissolved or dispersed therein as an active ingredient.

In various embodiments, the therapeutic composition is not substantially immunogenic when administered to a mammal or human patient for therapeutic purposes, unless so desired. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset, transplant rejection, allergic reaction, and the like. A pharmaceutically acceptable carrier will not promote the raising of an immune response to an agent with which it is admixed, unless so desired. The preparation of a composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared.

A transplant composition for humans may include one or more pharmaceutically acceptable carrier or materials as excipients. In contrast, a cell culture composition (not for human transplant) typically will use research reagents like cell culture media as an excipient. iNPCs could also be administered in an FDA-approved matrix/scaffold or in combination with FDA-approved drugs appropriate for a particular disease or condition (e.g., Riluzole (Rilutek®, manufactured by Sanofi-Aventis, LLC®) or Edaravone (Radicava®, manufactured by Mitsubishi Tanabe Pharma Corporation®)).

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

As used herein, the terms “transplanting” or “engraftment” is used in the context of the placement of cells, e.g. iNPCs or neurospheres, as provided herein into a subject, by a method or route which results in at least partial localization of the introduced cells at a desired site, such as a site of injury or repair, such that a desired effect(s) is produced. The cells e.g. iNPCs, or their differentiated progeny (e.g. astrocytes etc.) can be implanted directly to the spinal cord or brain, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the iNPCs is desired as adult neural progenitors do not proliferate to an extent that the spinal cord can heal from an acute injury or disease comprising cell death. In other embodiments, the cells can be administered via an indirect systemic route of administration, such as an intraperitoneal or intravenous route.

As used herein, the terms “administering,” “introducing” and “transplanting” are used interchangeably in the context of the placement of cells, e.g. iNPCs, as described herein into a subject, by a method or route which results in at least partial localization of the introduced cells at a desired site, such as a site of injury or repair or disease, such that a desired effect(s) is produced.

In some embodiments, the iNPCs or progeny thereof being administered according to the methods described herein comprises allogeneic cells or their obtained from one or more donors. As used herein, “allogeneic” refers to a cell obtained from or derived from (e.g., differentiated from) one or more different donors of the same species, where the genes at one or more loci are not identical. For example, NPCs being administered to a subject can be derived from umbilical cord blood obtained from one more unrelated donor subjects, or from one or more non-identical siblings. In some embodiments, syngeneic cell populations can be used, such as those obtained from genetically identical animals, or from identical twins. In other embodiments of this aspect, the cells are autologous cells; that is, the cells are obtained or isolated from a subject (or derived from) and administered to the same subject, i.e., the donor and recipient are the same.

In some embodiments, the cells useful for the compositions described herein are derived from an autologous source. Since the iNPCs (or their differentiated progeny) provided herein are essentially derived from an autologous source, the risk of engraftment rejection or allergic responses is reduced compared to the use of cells from another subject or group of subjects. In some embodiments, the cells useful for engraftment provided herein are derived from non-autologous sources. In addition, the use of iPSCs negates the need for cells obtained from an embryonic source. Thus, in one embodiment, the stem cells used to generate iNPCs for use in the compositions and methods described herein are not embryonic stem cells. In other embodiments, the iNPCs for use in the compositions and methods described herein are GDNF-expressing iNPCs.

### Method of Treating a Disease

Described herein is a method of treatment, including administering a quantity of iPSC-derived NPCs (iNPCs) to a subject afflicted with a disease or condition, wherein the cells express a therapeutic protein or peptide, and further wherein the cells, therapeutic protein or peptide, or both, are capable of treating the disease or condition. In other embodiments, the cells are iPSC-derived NPCs. In other embodiments, the cells are GDNF-expressing iNPCs.

Described herein is a method, including administering a quantity of iPSC-derived NPCs to a subject afflicted with a neurodegenerative disease, wherein the cells inducibly express a neurotrophic factor capable of treating the disease. In other embodiments, the iPSC-derived NPCs express a genomically integrated expression cassette introduced by nucleofection, the expression cassette including a constitutive promoter, an inducible, bi-directional polycistronic promoter including a tet response element, and a sequence encoding glial derived neurotrophic factor (GDNF). In various embodiments, the vector includes at least one homologous recombination sequence. In other embodiments, the method includes administration of tetracycline, an analog or derivative thereof. In other embodiments, the vector excludes a polypeptide reporter sequence. In other embodiments, the vector excludes a green fluorescent protein (GFP) sequence.

Described herein is a method of treatment, including: administering a quantity of cells to a subject afflicted with a disease or condition, wherein the cells express a therapeutic protein or peptide, and further wherein the cells, therapeutic protein or peptide, or both, are capable of treating the disease or condition. In each and all of the aforementioned embodiments of the method, the cells iPSC-derived NPCs. In each and all of the aforementioned embodiments of the method, the cells express an expression cassette from one or more vectors. In each and all of the aforementioned embodiments of the method, the cells expressing the expression cassette from the one or more vectors have been nucleofected, transfected, or electroporated. In each and all of the aforementioned embodiments of the method, the one or more vectors includes a piggyBac vector, a pBase vector, or both. In each and all of the aforementioned embodiments of the method, the piggyBac vector includes at least two promoters, wherein at least one promoter is inducible. In each and all of the aforementioned embodiments of the method, the at least one inducible promoter is polycistronic. In each and all of the aforementioned embodiments of the method, the at least one inducible, polycistronic promoter is bi-directional. In each and all of the aforementioned embodiments of the method, the expression cassette is genomically integrated. In each and all of the aforementioned embodiments of the method, the expression cassette encodes the therapeutic protein or peptide. In each and all of the aforementioned embodiments of the method, the therapeutic protein or peptide includes a neurotrophic factor. In each and all of the aforementioned embodiments of the method, the neurotrophic factor includes glial derived neurotrophic factor (GDNF). In each and all of the aforementioned embodiments of the method, the disease or condition is a neurodegenerative disease. In each and all of the aforementioned embodiments of the method, the neurodegenerative disease is amyotrophic lateral sclerosis (ALS). In each and all of the aforementioned embodiments of the method, administering a quantity of cells includes injection. In each and all of the aforementioned embodiments of the method, the method includes administration of tetracycline, an analog or derivative thereof.

Also described herein is a method, including administering a quantity of iPSC-derived NPCs to a subject afflicted with a neurodegenerative disease, wherein the cells inducibly express a neurotrophic factor capable of treating the disease. In each and all of the aforementioned embodiments of the method, the iPSC-derived NPCs express a genomically integrated expression cassette introduced by nucleofection, the expression cassette including: a constitutive promoter, an inducible, bi-directional polycistronic promoter including a tet response element, and a sequence encoding glial derived neurotrophic factor (GDNF). In each and all of the aforementioned embodiments of the method, the method includes administration of tetracycline, an analog or derivative thereof.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions and formulations as disclosed herein will be decided by the attending physician within the scope of sound medical judgment. The exact amount required will vary depending on factors such as the type of disease being treated.

Provided herein are methods for treating a disease, disorder, spinal cord injury, amyotrophic lateral sclerosis (ALS) or infection comprising administering iNPCs to a subject in need thereof. In some embodiments, methods and compositions are provided herein for the prevention of an anticipated disorder e.g., ALS.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions and formulations as disclosed herein will be decided by the attending physician within the scope of sound medical judgment. The exact amount required will vary depending on factors such as the type of disease being treated.

The term “effective amount” as used herein refers to the amount of a population of iNPCs needed to alleviate at least one or more symptoms of a disease or disorder, including but not limited to an injury, disease, or disorder. An “effective amount” relates to a sufficient amount of a composition to provide the desired effect, e.g., protection of motor neurons following neurodegenerative injury, replacement of neural progenitor cell populations, etc. The term “therapeutically effective amount” therefore refers to an amount of iNPCs or a composition such cells that is sufficient to promote a particular effect when administered to a typical subject, such as one who has, or is at risk for, a neurodegenerative disease or disorder. An effective amount as used herein would also include an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting the brain, spinal cord, or neurons prior to administering the cells according to the methods described herein. In some embodiments, the subject is first diagnosed as being at risk of developing a disease (e.g., ALS) or disorder prior to administering the cells.

For use in the various aspects described herein, an effective amount of iNPCs comprises at least 1×10³, at least 1×10⁴, at least 1×10⁵, at least 5×10, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more iNPCs. The effective amount of iNPCs will depend on the size and the area of the engraftment site. For example, for the engraftment of iNPCs into the optic nerve, about 1000 to 10,000 iNPCs can be used. By contrast, engraftment of iNPCs into a spinal cord may require many more cells, on the order of about 1×10⁶ to about 5×10⁹ or more iNPCs. A skilled practitioner can determine the number of cells that are needed for a given engraftment procedure.

In some embodiments, a composition comprising iNPCs is treated with any one or more of the vectors provided herein and permits protection of motor neurons in the target tissue (e.g., spinal cord) at an efficiency at least 20% greater than the engraftment when such cells are administered alone; in other embodiments, such efficiency is at least 30%, at least 40%, at least 50%, at least 60%, at least 70%, at least 80%, at least 90%, at least 1-fold, at least 2-fold, at least 5-fold, at least 10-fold, at least 100-fold or more than the efficiency of engraftment when iNPCs are administered alone without the vectors provided herein (e.g., vectors comprising a nucleic acid sequence encoding GDNF).

In some embodiments, a composition comprising iNPCs is treated with any one or more of the vector provided herein and permits engraftment of the cells in the target tissue (e.g., spinal cord) at an efficiency at least 20% greater than the engraftment when such cells are administered alone; in other embodiments, such efficiency is at least 30%, at least 40%, at least 50%, at least 60%, at least 70%, at least 80%, at least 90%, at least 1-fold, at least 2-fold, at least 5-fold, at least 10-fold, at least 100-fold or more than the efficiency of engraftment when iNPCs are administered alone without the vectors described herein (e.g., vectors comprising a nucleic acid sequence encoding GDNF).

In some embodiments, an effective amount of iNPCs are administered to a subject by intraspinal administration or delivery. In some embodiments, an effective amount of iNPCs is administered to a subject by systemic administration, such as intravenous administration

The phrases “systemic administration,” “administered systemically”, “peripheral administration” and “administered peripherally” are used herein refer to the administration of a population of iNPCs other than directly into a target site, tissue, or organ such that it enters, instead, the subject's circulatory system.

The choice of formulation will depend upon the specific composition used and the number of iNPCs to be administered; such formulations can be adjusted by the skilled practitioner. However, as an example, where the composition is iNPCs in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, the formulation can comprise a scaffold, such as a biodegradable scaffold.

In some embodiments, additional agents to aid in treatment of the subject can be administered before or following treatment with the iNPCs as described. Such additional agents can be used to prepare the target tissue for administration of the progenitor cells. Alternatively, the additional agents can be administered after the iNPCs to support the engraftment and growth of the administered cell into the spinal cord, or other desired administration site. In some embodiments, the additional agent comprises growth factors, such as FGF, EGF, or LIF.

The efficacy of treatment can be determined by the skilled clinician. However, a treatment is considered “effective treatment,” as the term is used herein, if any one or all of the symptoms, or other clinically accepted symptoms or markers of disease, e.g., neurodegenerative disease, ALS, spinal cord injury and/or a disorder are reduced, e.g., by at least 10% following treatment with a composition comprising human iNPCs as described herein. Methods of measuring these indicators are known to those of skill in the art and/or described herein.

Indicators of a neurodegenerative disease or disorder, or neurological injury include functional indicators or parameters, e.g., muscle weakness, problems with coordination, stiff muscles, loss of muscle, muscle spasms, or overactive reflexes, fatigue or feeling faint, difficulty speaking or vocal cord spasm, difficulty swallowing, drooling, lack of restraint, mild cognitive impairment, severe constipation, severe unintentional weight loss, shortness of breath, or difficulty raising the limbs/extremities (e.g. foot or arm) among others.

Non-limiting examples of clinical tests that can be used to assess neurological functional parameters include: an electromyogram (EMG), MRI, nerve conduction study, blood tests, spinal tap, or muscle biopsy.

Where necessary or desired, animal models of injury or disease can be used to gauge the effectiveness of a particular composition as described herein. For example, a genetic rodent model of ALS, SOD1^{G93A} described in detail in the working examples and e.g., in Gurney M E et al. Motor neuron degeneration in mice that express a human Cu, Zn superoxide dismutase mutation. Science 1994; 264: 1772-1775, which is incorporated herein by reference in its entirety.

### Some Selected Definitions

One skilled in the art will recognize many methods and materials similar or equivalent to those described herein, which could be used in the practice of the present invention. Indeed, the present invention is in no way limited to the methods and materials described.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in biology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012)(ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, Revised, J. Wiley & Sons (New York, NY 2006); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present, so long as the selected coding sequence is capable of being replicated, transcribed and translated in an appropriate host cell.

The term “promoter region” is used herein in its ordinary sense to refer to a nucleotide region including a DNA regulatory sequence, wherein the regulatory sequence is derived from a gene which is capable of binding RNA polymerase and initiating transcription of a downstream (3′-direction) coding sequence.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In the context of encoding sequences, promoters, and other genetic elements provided herein, the term “heterologous” indicates that the element is derived from a genotypically distinct entity from that of the rest of the entity to which it is being compared. For example, a promoter or gene introduced by genetic engineering techniques into a cell provided herein is said to be a heterologous polynucleotide. An “endogenous” genetic element is an element that is in the same place in the chromosome where it occurs in nature, although other elements may be artificially introduced into a neighboring position.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease.

The term “marker” as used herein is used to describe a characteristic and/or phenotype of a cell. Markers can be used for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

As used herein, the term “scaffold” refers to a structure, comprising a biocompatible material that provides a surface suitable for adherence and proliferation of cells. A scaffold can further provide mechanical stability and support. A scaffold can be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 100% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount, for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the term “modulates” refers to an effect including increasing or decreasing a given parameter as those terms are defined herein.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with a disease, or a biological sample that has not been contacted with a composition, polypeptide, or nucleic acid encoding such polypeptide as disclosed herein).

As used herein, an “appropriate control” refers to an untreated, otherwise identical cell or population (e.g., a biological sample that was not contacted by an agent or composition described herein, or not contacted in the same manner, e.g., for a different duration, as compared to a non-control cell).

As used herein, the term “phenotypic characteristic,” as applied to in vitro differentiated cells (e.g., iNPCs), or culture of in vitro-differentiated cells, refers to any of the parameters described herein as measures of cell function. A “change in a phenotypic characteristic” as described herein is indicated by a statistically significant increase or decrease in a functional property with respect to a reference level or appropriate control.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The word “or” is intended to include “and” unless the context clearly indicates otherwise.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Although methods and materials similar or equivalent to those described herein can be used in the practice or testing of this disclosure, suitable methods and materials are described below. The abbreviation, “e.g.” is derived from the Latin Exempli gratia, and is used herein to indicate a non-limiting example. Thus, the abbreviation “e.g.” is synonymous with the term “for example.”

Exemplary embodiments of the various aspects described herein can be defined as follows:

Embodiment 1: A method to generate induced pluripotent stem cell (iPSC)-derived neuronal progenitor cells (iNPCs), comprising: (i) providing a quantity of stem cells; (ii) culturing the stem cells in the presence of a RHO kinase inhibitor (ROCK inhibitor); (iii) generating a monolayer of cells; (iv) culturing the cells in the presence of one or more of LDN and SB; and (v) culturing the cells in the presence of one or more of FGF, EGF and LIF to generate neural progenitor cells (NPCs).

Embodiment 2: The method of embodiment 1, wherein the stem cells are induced pluripotent stem cells (iPSCs).

Embodiment 3: The method of any one of the preceding embodiments, wherein the NPCs are induced pluripotent stem cell derived NPCs (iNPCs).

Embodiment 4: The method of any one of the preceding embodiments, wherein generating the monolayer comprises shaking the cultured iPSCs.

Embodiment 5: The method of any one of the preceding embodiments, wherein the cells are cultured in LDN and SB for about 7 to about 13 days.

Embodiment 6: The method of any one of the preceding embodiments, wherein the cells are cultured in: (i) about 4.75 μg/mL to about 5.75 μg/mL LDN; and/or (ii) about 0.5 μM to about 4 μM SB for about 7 to about 13 days.

Embodiment 7: The method of any one of the preceding embodiments, wherein the LDN is LDN193189 and the SB is SB-431542.

Embodiment 8: The method of any one of the preceding embodiments, wherein the ROCK inhibitor is selected from the group consisting of: Fasudil, Ripasudil, Netarsudil, RKI-1447, Y-27632, GSK429286A, Y-30141, or any combination thereof.

Embodiment 9: The method of any one of the preceding embodiments, wherein the ROCK inhibitor is Y-27632.

Embodiment 10: The method of any one of the preceding embodiments, wherein the cells are cultured in about 5 μM Y-27632 for at least about 7 to about 16 days.

Embodiment 11: The method of any one of the preceding embodiments, wherein the cells are cultured in FGF, EGF, and LIF for at least about 8 hours to 16 days.

Embodiment 12: The method of any one of the preceding embodiments, wherein the cells are cultured in 100 ng/mL of FGF, 100 ng/mL of EGF, and 100 ng/mL of LIF for at least about 3 days to about 16 days.

Embodiment 13: The method of any one of the preceding embodiments, wherein the iPSC-derived NPCs (iNPCs) are aggregated as neurospheres.

Embodiment 14: The method of any one of the preceding embodiments, wherein the iPSC-derived NPCs are engrafting iPSC-derived NPCs.

Embodiment 15: The method of any one of the preceding embodiments, wherein the cells are contacted with one of more vectors.

Embodiment 16: The method of any one of the preceding embodiments, wherein the cells are contacted with a vector comprising: (a) an expression cassette, comprising: (i) a constitutive promoter, (ii) an inducible, bi-directional polycistronic promoter comprising a tet responsive element, and (iii) a sequence encoding a protein or peptide; (b) two transposon elements, wherein the two transposon elements flank the expression cassette; and (c) at least one homologous recombination sequence.

Embodiment 17: The method of any one of the preceding embodiments, wherein the cells are contacted with a vector comprising a nucleic acid sequence encoding a neurotrophic factor.

Embodiment 18: The method of any one of the preceding embodiments, wherein the neurotrophic factor is glial cell line-derived neurotrophic factor (GDNF).

Embodiment 19: A method, comprising: (i) providing a quantity of iPSC-derived NPCs made by the method of any previous embodiment; and (ii) introducing at least two vectors into the iPSC-derived NPCs.

Embodiment 20: The method of any one of the preceding embodiments, wherein the introducing at least two vectors comprises one or more of: nucleofection, transfection and electroporation.

Embodiment 21: The method of any one of the preceding embodiments, wherein the at least two vectors comprise a piggyBac vector and a pBase vector.

Embodiment 22: The method of any one of the preceding embodiments, wherein at least one vector comprises a viral vector.

Embodiment 23: The method of any one of the preceding embodiments, wherein the vector is an AAV or a lentiviral vector.

Embodiment 24: The method of any one of the preceding embodiments, wherein the piggyBac vector comprises: (a) an expression cassette, comprising: (i) a constitutive promoter, (ii) an inducible, bi-directional polycistronic promoter comprising a tet responsive element, and (iii) a sequence encoding a protein or peptide; (b) two transposon elements, wherein the two transposon elements flank the expression cassette; and (c) at least one homologous recombination sequence.

Embodiment 25: The method of any one of the preceding embodiments, wherein the protein or peptide comprises a neurotrophic factor.

Embodiment 26: The method of any one of the preceding embodiments, wherein the neurotrophic factor comprises glial derived neurotrophic factor (GDNF).

Embodiment 27: The method of any one of the preceding embodiments, wherein the homologous recombination sequence comprises a sequence capable of targeting a genomic safe harbor.

Embodiment 28: The method of any one of the preceding embodiments, wherein the genomic safe harbor is one of: the adeno-associated virus site 1 (AAVS1), the chemokine (C—C motif) receptor 5 (CCR5) gene, human ortholog of the mouse Rosa26 locus.

Embodiment 29: The method of any one of the preceding embodiments, wherein the neuronal progenitor cells are engrafting neuronal progenitor cells.

Embodiment 30: A quantity of cells made by the method of any preceding embodiment.

Embodiment 31: The method or quantity of cells of any one of the preceding embodiments, wherein the cells express a genomically integrated expression cassette.

Embodiment 32: The method or quantity of cells of any one of the preceding embodiments, wherein the genomically integrated expression cassette is at a genomic safe harbor.

Embodiment 33: A method, comprising: (i) providing a quantity of iPSC-derived NPCs made by the method of any one of the preceding embodiments; and (ii) introducing at least one vector into the iPSC-derived NPCs.

Embodiment 34: The method of any one of the preceding embodiments, wherein at least one vector comprises one or more of: nucleofection, transfection and electroporation.

Embodiment 35: The method of any one of the preceding embodiments, wherein the at least one vector comprises: (a) an expression cassette, comprising: (i) a constitutive or inducible promoter operably linked to a sequence encoding a protein or peptide; and (ii) at least one homologous recombination sequence.

Embodiment 36: The method of any one of the preceding embodiments, wherein the protein or peptide comprises a neurotrophic factor.

Embodiment 37: The method of any one of the preceding embodiments, wherein the neurotrophic factor comprises glial derived neurotrophic factor (GDNF).

Embodiment 38: The method of any one of the preceding embodiments, wherein the constitutive promoter is 3-phosphoglycerate kinase (PGK promoter).

Embodiment 39: The method of any one of the preceding embodiments, wherein the homologous recombination sequence comprises a sequence capable of targeting a genomic safe harbor.

Embodiment 40: The method of any one of the preceding embodiments, wherein the genomic safe harbor is one of: the adeno-associated virus site 1 (AAVS1), the chemokine (C—C motif) receptor 5 (CCR5) gene, human ortholog of the mouse Rosa26 locus.

Embodiment 41: The method of any one of the preceding embodiments, wherein the inducible promoter comprises a promoter regulated by a tetracycline-class antibiotic.

Embodiment 42: The method of any one of the preceding embodiments, wherein the tetracycline-class antibiotic comprises doxycycline.

Embodiment 43: The method of any one of the preceding embodiments, wherein the inducible promoter is regulated by a reverse tetracycline-controlled transactivator (rtTA) or a tet-On advanced transactivator (rtTA2S-M2).

Embodiment 44: The method of any one of the preceding embodiments, wherein the reverse tetracycline-controlled transactivator (rtTA) or a tet-On advanced transactivator (rtTA2S-M2) promotes the expression of GDNF in a cell.

Embodiment 45: The method of any one of the preceding embodiments, wherein the iPSC-derived NPCs are engrafting iPSC-derived NPCs.

Embodiment 46: A quantity of cells made by the method any preceding embodiment, wherein the iPSC-derived NPCs express a genomically integrated expression cassette.

Embodiment 47: The quantity of cells of any preceding embodiment, wherein the genomically integrated expression cassette is at a genomic safe harbor.

Embodiment 48: The quantity of cells of any preceding embodiment, wherein the genomic safe harbor is AAVS1.

Embodiment 49: A transplant composition comprising the quantity of cells of any preceding embodiment and a pharmaceutically acceptable carrier.

Embodiment 50: The transplant composition of any preceding embodiment, wherein the composition is engrafted into the spinal cord of the subject.

Embodiment 51: A transplant composition of any preceding embodiment for use as a treatment for a neurodegenerative disease.

Embodiment 52: The transplant composition of any preceding embodiment, wherein the neurodegenerative disease is amyotrophic lateral sclerosis (ALS).

Embodiment 53: A method of treating ALS, the method comprising: (a) administering to the subject iNPCs made by the method of any preceding embodiment, and optionally (b) administering to the subject an additional treatment for ALS.

Embodiment 54: The method of any preceding embodiment, wherein the additional treatment is one or more of Riluzole or Edaravone.

The various methods and techniques described herein provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein.

A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to induced pluripotent stem cells (iPSCs), differentiated iPSCs including neural progenitor cells, vectors used for manipulation of the aforementioned cells, methods and compositions related to use of the aforementioned compositions, techniques and composition and use of solutions used therein, and the particular use of the products created through the teachings of the invention. Various embodiments of the invention can specifically include or exclude any of these variations or elements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

It is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

This invention is further illustrated by the following examples which should not be construed as limiting.

### EXAMPLES

Described herein are non-limiting examples of the claimed invention.

### Example 1: Development of iNPC-GDNF^{DOX} Cell Product Through Generation of a Master IPSC Line Engineered to Produce Gdnf Under a Tetracycline Inducible Promotor

Induced-pluripotent stem cells (iPSCs) present a safe, renewable, and scalable source for a cell therapy product that can be differentiated into the desired neural progenitor cell. Because iPSCs can be clonally expanded, it is possible to generate a master iPSC line that contains a single copy of the GDNF transgene inserted into a safe genomic locus such as AAVS1, under an inducible promotor. The derived iNPC-GDNF cells can then uniformly express GDNF when provided with tetracycline and can be readily expandable to scales sufficient for further testing and clinical use.

In this study, a iPSC master cell line was generated that is engineered to produce GDNF under a tetracycline regulated promotor. With an optimized differentiation protocol and an engineered line for controlled GDNF expression, a preclinical cell lot of the iNPC-GDNF^{dox} can be expanded and banked for efficacy and safety studies.

Lentiviral transduction, such as that used in CNS10-NPC-GDNF, results in heterogenous copy number and random genomic insertion. Because iPSCs can be expanded from a single clone, establishing gene edited lines with a single GDNF construct inserted into the AAVS1 safe landing site is possible.

Gene-edited iPSC lines are notorious for silencing engineered constructs upon differentiation^{36,37}. Because the AAVS1 site remains in a region of open chromatin, constructs inserted here are better able to retain expression in differentiated progeny^{38,39}. Specifically, the AAVS1 locus is safe for insertion of engineered constructs because insertion results in minimal disruption of endogenous cellular processes⁴⁰. CNS10-NPC-GDNF secrete GDNF that can protect motor neurons, but this expression is constitutive and so the timing and dose of GDNF cannot be regulated. In this study, the inventors engineered an iPSC line to express GDNF that is regulatable, much like that used in collaborative studies²⁷.

Two constructs were generated with either a tetracycline inducible promotor driving GDNF (V1), or a constitutive promotor (FIG. 6A). These constructs were targeted for insertion into the AAVS1 locus using previously established TALENs and a homologous recombination approach (FIG. 7). iPSC lines that harbor each construct were differentiated into iNPCs and then tested in vitro using ELISA to detect GDNF secretion into the media. At 24 hours, non-transduced iNPCs did not produce GDNF yet iNPCs harboring the constitutive GDNF construct produced similar levels of GDNF to both lentivirally transduced iNPCs and CNS10-NPC-GDNF cells (FIG. 6B). iNPCs with the V1 inducible construct treated with 0.6 μM doxycycline (dox; a tetracycline analogue) showed expression of GDNF. The inducible construct was tightly regulated, with attenuation of GDNF production by 72 hours after dox withdrawal (FIG. 6C). Importantly, without dox treatment, GDNF levels from the transduced iNPCs remained at non-transduced levels.

Despite promising results from the in vitro data, the V1 tetracycline-inducible construct did not function in transplanted cells (FIG. 6D). Additionally, the initial construct was large at 10.7 kb and contained many elements that are not appropriate for therapeutic application²⁷. Therefore, the inventors designed a simplified construct from the clean vector backbone available from CloneTech™ (FIG. 6A, V2). This new tetracycline inducible system (V2) is much smaller at 5.7 kb and can result in cleaner and more efficient expression of the GDNF transgene.

To engineer an iPSC line with a single copy of GDNF expressed under control of the inducible promoter, a TALEN methodology can be used to target the new V2 construct into the AAVS1 locus. Further, incorporation of the GDNF construct into the iPSC rather than iNPCs eliminates the need for a dissociation/lentiviral transduction step later on in culture, thus further increasing the expansion potential.

Efficient integration of a targeted construct into the AAVS1 loci using this approach has been demonstrated⁴¹. The same TALEN constructs can be used to insert the V2 inducible GDNF transgene into the iPSC line used for evaluation of iNPC protocols earlier. iPSCs in suspension can be electroporated with the Right and Left TALEN constructs and the inducible GDNF construct. The cells can be plated at low density and selected to establish clonal lines. Clones that express the desired construct can be selected based on transient GFP expression, and integration into the AAVS1 site can be confirmed through Sanger sequencing. After confirmation, these iPSC clones can serve as material for deriving the inducible iNPC product (iNPC-GDNF^{dox}).

Once an iPSC line harboring the V2 inducible GDNF construct has been established, the cells can be differentiated and characterized, e.g., by qPCR for established NPC genes (Nestin, VIM, TUBB3, MAP2, APQ4, S100β and GFAP), plate-downs and immunocytochemistry (ICC) to establish the ability of the NPCs to generate astrocytes, as well as ICC and ELISAs for GDNF to characterize the in vitro levels of GDNF production and dynamics of tetracycline regulation.

Once a batch of iNPC-GNDFdox has been differentiated and characterized, the cells can be expanded and banked. In parallel, a bank of constitutively expressing GDNF iNPCs can be developed using the same process (iNPC-GDNF^{CONST}). Previous work with the current iNPC differentiation has shown that iNPCs are amenable to scalable culture methods, which is critical for downstream development into cellular therapeutics.

In addition to the iPSC lines discussed above, a constitutive GDNF iPSC line has been generated, as well as differentiated and banked the iNPC-GDNF^{CONST} cells. These iNPC-GDNF^{CONST} cells can be used for efficacy and safety testing as described below. The cell products described are capable of scale production as described below.

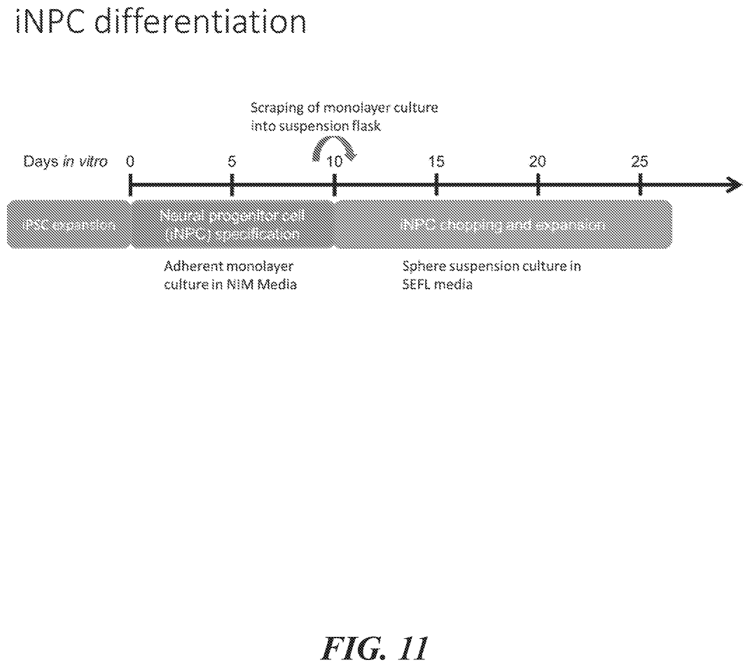

Efficacy and Safety testing: To conduct and safety testing, a batch of roughly two-hundred million iNPCs can be generated. Through the employment of scaling bioreactors and a novel passaging method, this batch size is easily achievable. Traditionally, neural progenitor cells are expanded as either a monolayer or in suspension as aggregate cultures. Single cell passaging of either culture modality is not ideal as this passage method can lead to early cell senesce, which limits expansion potential, or can induce the cells to differentiate^{13,42,43}. Mechanical chopping has been successfully used to expand both fetal and iPSC-derived neural progenitor cells to scales suitable for early phase clinical trials. However, this method is time-consuming, labor-intensive, and challenging to implement at larger scales. A method of mechanical passaging by inserting a cutting mesh made from ultra-fine tungsten wire with 200 μm square spaces was developed to aid in downstream manufacturing of the cell product described herein. Since the mesh is 98% open, fluid flow is minimally impeded which allows large volumes of media and cells to flow past, the large spheres are then cut as they pass through the mesh. The resulting sphere sections are roughly 200 μm square segments similar to those from the traditional mechanical chopping method. In addition to being significantly less time consuming and requiring far fewer operator manipulations, this new chopping method can be implemented in-line, eliminating the need for external handling of the cells altogether. This enables the use of scaling bioreactor cultures where the culture volume can be increased each passage as opposed to the scale-out culture methods employed to generate CNS10-NPC-GNDF cells where the number of flasks is increased each passage. Using even small bioreactor cultures and this novel mechanical passaging technique, sufficient quantities of iNPC-GDNFdox/CONST for all downstream assays can be rapidly produced (FIG. 11).

### Example 2: Efficacy of iNPC-GDNF^{dox} Transplanted in the Lumbar Spinal Cord and Motor Cortex of the SOD1G93A ALS Transgenic Rat

The SOD1^{G93A} rats are a well-characterized model of ALS^{44,45} and have been extensively used for cell transplantation studies^{8,12}, as well as in preclinical studies for IND filing for the fetal-derived CNS10-NPC-GDNF cells. Much like human pathology, the location of disease onset is unpredictable in this model, with overt paresis progressing to complete paralysis in the hindlimbs and/or forelimbs. Atrophy of the trunk and neck muscles can also be observed in some animals. This slow deterioration and disease progression can be evaluated using behavioral measurements, such as body weight and the Basso, Beattie, and Bresnahan (BBB) scale. Histological analysis of these rats shows corticospinal motor neuron and spinal motor neuron loss, and degeneration of the neuromuscular junction^{14,46}. Thus, this model is particularly suitable to determine the dosing and efficacy of iNPC-GDNF^{dox} in protecting neurons that are susceptible to ALS. This aim focuses on whether the iNPC-GDNF^{dox} product can provide protective benefits in an ALS rodent model while under the control of an inducible promoter.

Dox dosing scheme: In a previous study, regulation of iPSC-derived neural progenitor transplants in mouse cortex was achieved by delivering 15 μg dox/gram every 3-4 days via oral gavage²⁷. As determined by a luciferase assay, the effects of dox dosage were seen within a week of administration. Because the efficacy of iNPC-GDNF^{dox} is the primary goal of this study, only a simple delivery of “on dox” or “off dox” needs to be evaluated. The in vivo dynamics of GDNF expression can be evaluated as well as more complex dosing regimens such as pulsed expression and attenuation. Here, wild type (WT) rats can be used. Each animal can receive iNPC-GDNF^{dox} transplants into the lumbar spinal cord, unilaterally to allow for comparison to the contralateral side of each spinal cord section. Each animal can receive 3 injections of 10K cells at sites spaced 1 mm apart. Based on the in vitro GDNF ELISA from iNPCs harboring the V1 construct, an effect size of 4.91 between dox treated and untreated cells was seen, allowing for 99% power to detect a difference in effect using a group size of 3 animals. After transplantation and recovery from surgery, dox can be administered to the rats in one of two ways, with each group evaluating three different concentrations, totaling 18 animals (Table 3). The first group can receive dox using oral gavage at the following concentrations: 15 μg, 20 μg and 30 μg per dose. The second group can receive dox in drinking water at the following concentrations: 0.2 mg/mL, 2 mg/mL and 5 mg/mL. A study evaluating the efficacy of dox when delivered in drinking water showed dox to be stable in animal water bottles for up to 14 days⁴⁷, so here the dox-water can be replaced with fresh mixtures twice a week. After 4 weeks of gavage every 3-4 days or constant access to dox-water, the animals can be sacrificed and assessed for GDNF expression and graft survival using immunohistochemistry (IHC) on spinal cord tissue. During treatment, animals can be observed using behavior measurements (body weight and observation) to ensure recovery and lack of injury to the spinal cord following surgery. The exact volume of drinking water can be measured at each change to estimate the amount being consumed by each rat. The rats can receive daily, alternating intraperitoneal injections (IP) of 10 mg/kg cyclosporine for immunosuppression to avoid graft rejection, beginning 3 days prior to surgery. Each dox administration method and concentration can be scored according to the amount of GDNF detected in spinal cord sections and compared to the percent human nuclei or cytoplasmic marker that co-localize with DAPI, which is indicative of graft survival.

TABLE 3
Experimental groups for optimal dox delivery
for iNPC-GDNFdox in Wild-Type (WT) animals.

| | Administration by drinking H2O | | | Administration by oral gavage | | |
|---|---|---|---|---|---|---|
| | [mg/mL] | | | [μg] | | |
| Dox | 0.2 | 2 | 5 | 15 | 20 | 30 |
| WT rats | 3 | 3 | 3 | 3 | 3 | 3 |

Efficacy of iNPC-GDNF in protecting neurons in SOD1^{G92A} rat model of ALS: Neuroprotection of spinal motor neurons after transplantation of CNS10-NPC-GDNF therapy to the lumbar spinal cord in ALS and aging has been shown^{8,12,26}. Additional work has also shown that viral knockdown of mutant SOD1 in the motor cortex of the SOD1^{G}3A rat model resulted in delayed disease onset and extended survival¹⁴. Transplantation of CNS10-NPC-GDNF into the motor cortex in this ALS model also protected both upper and lower motor neurons, delayed disease pathology and extended survival of the ALS rats¹⁵. Both studies suggested that dysfunction of the upper motor neurons and cortex may contribute significantly to events that lead to motor neuron death in the brain and spinal cord, and the consequent paralysis in ALS. Without being bound by a particular theory, iNPC-GDNF^{dox} cells transplanted into the (a) lumbar spinal cord, (b) motor cortex, and (c) both sites of the SOD1^{G93A} rat model, can provide neuroprotection similar to that observed with the CNS10-NPC-GDNF product.

Based on previous experience with the SOD1^{G93A} rat model, all animals in these efficacy studies can receive transplants of iNPC-GDNF^{dox} cells at 70+/−5 days old. The method of dox administration, and minimal-effective concentration, discussed above, can be used after the animals have been given a week to recover post-surgery.

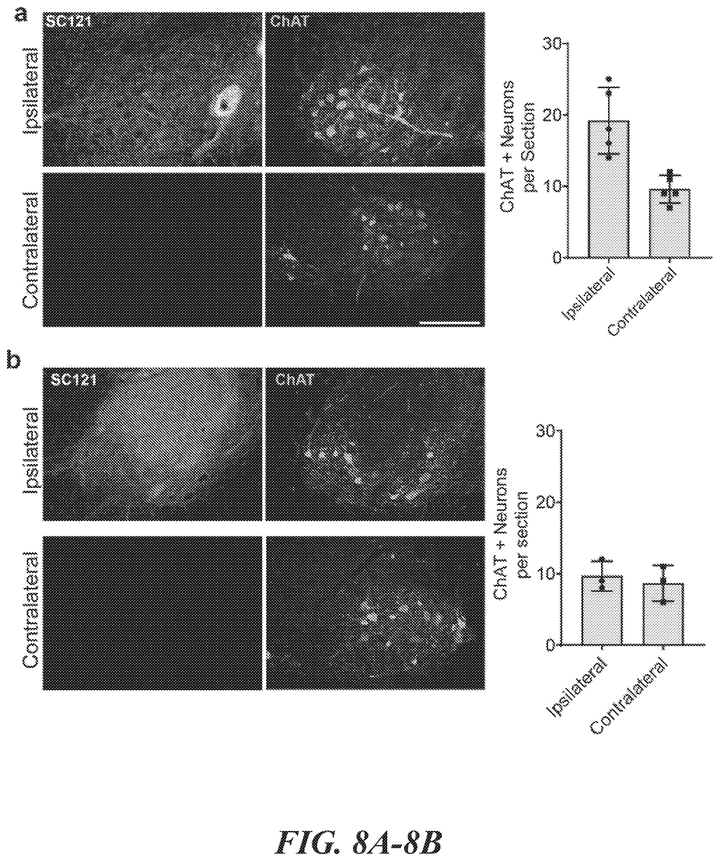

iNPC-GDNFdox transplantation into the lumbar spinal cord: In previous studies, iNPCs that have been transduced with lentivirus to express GDNF showed neuroprotection in the lumbar spinal cord of SOD1^{G93A} ALS rats 50 days after transplantation. In these animals, transplanted doses of 10K cells per site were effective at preserving host ChAT+ motor neurons, but doses of 50K cells per site resulted in no neuroprotection and displacement of the host neurons from the over-dense grafts (FIG. 8A-8B).

In determining the effective dose of iNPC-GDNFdox cells, male SOD1G93A rats can be used. The statistical justification of animals to be used in each cohort is based upon previously published and unpublished data with the CNS10-GDNF cells8,12,14,15,48. An average of 350±33 (standard error of the mean, SEM) of large, ChAT+ motor neurons (>700 μm2) are expected to remain in the spinal cord of vehicle treated SOD1G93A rats at disease onset. Therefore, sample sizes of 10 animals per vehicle group and 15 per treatment groups are used in order to achieve 80% power in a repeated measures analysis (ipsilateral versus contralateral measurements), with an anticipated effect size of 0.58. The treated groups encompass three gradually increasing concentrations of cells to be transplanted per site, from 10 thousand (D1) up to 30 thousand cells (D3), with each site receiving 2 μL of volume (Table 4). The group D3 which receives the highest dose of cells per site includes an additional 10 animals that will not receive dox administration after transplantation (for a total of 25 animals). Sites in the lumbar spinal cord can be spaced 1 mm apart, with 5 sites in total. Transplants can be unilateral (all on the same side per spinal cord), with the contralateral side serving as an internal control for each animal. The side of transplant can be chosen at random at the time of surgery and can be kept blinded from research staff until after completing histological analysis of the tissues and complete un-blinding of the data. SOD1^{G93A} rats will receive daily, alternating IP of 10 mg/kg cyclosporine for immunosuppression to avoid graft rejection. Following transplant, animals can be observed for detailed clinical examinations, including body weight, motor function of hindlimbs, morbidity and mortality. These observations can be fully blinded to the treatment groups. All animals can be sacrificed at disease onset as determined by a consecutive BBB score of 15 or less on either hindlimb or forelimb for further analysis.

TABLE 4
Experimental groups for lumbar spinal
cord transplantation iNPC-GDNF^{dox}.

| | | | Injection | |
|---|---|---|---|---|
| | Dose | Dose | concentration | |
| | (cells/animal) | [cells/site] | [cells/μL] | # of Animals |
| CTRL | N/A | N/A | N/A | 10 |
| D1 | 30k | 10k | 5,000 | 15 |
| D2 | 60k | 20k | 10,000 | 15 |
| D3 | 90k | 30k | 15,000 | 25* |

*10 animals will not be given dox as a transplantation control

Spinal cords can also be collected from each animal and sectioned serially for analysis by IHC. Motor neuron number, the degree of engraftment (determined by percentage of human nuclei and cytoplasm detected), and expression of GDNF can be evaluated using antibodies and stereology against each. The secondary goal of this aim is to evaluate the in vivo cell fate in a disease environment. To this end, grafted sections of spinal cord will also be evaluated using IHC to assess the cell type, such as astroglia or neural progenitor expression, and degree of integration or migration of the grafted cells. Efficacy of the iNPC-GDNF^{dox} can be scored by the percentage of motor neurons remaining compared to vehicle control, and contralateral regions of the same sections in treatment groups, and either no effect or an improved effect on behavioral data.

iNPC-GDNFdox transplantation into the motor cortex: Based on previous experience transplanting CNS10-NPC-GDNF into the motor cortex of SOD1^{G93A} rats, 2 μl injections are transplanted at 20 sites per animal (10 per hemisphere). Animals receive bilateral injections administered at a depth of 1.45 mm at lateral×anterior/posterior stereotaxic coordinates that encompass the motor cortex from Bregma loci as follows: (1) 2 mm×2 mm, (2) 2 mm×1 mm, (3) 2 mm×0 mm, (4) 2 mm×−1 mm, (5) 2 mm×−2 mm, (6) 3 mm 2 mm (7) 3 mm×1 mm, (8) 3 mm×0 mm, (9) 3 mm×−1 mm, (10) 3 mm×−2 mm. Animals can be separated into 4 groups, with 10 rats receiving vehicle control transplants (CTRL), and 3 groups of 15 rats each receiving gradually increasing cell doses, ranging from 400K cells to 2M cells per animal (Table 5). The highest treatment dose group D3 has an additional 10 animals which do not receive dox after transplantation, totaling 25 animals. All rats receive daily immunosuppression to avoid graft rejection via IP injection on alternating sides. Following transplant, animals can be observed for detailed clinical examinations, including body weight, motor and sensory function of hindlimbs, morbidity and mortality. These observations can be fully blinded from the treatment groups (CTRL, D1, D2 or D3), and whether animals are receiving dox treatment (i.e. D3 group includes 10 animals that are not administered dox). All animals can be sacrificed at disease onset as determined by a consecutive BBB score of 15 or less on either hindlimb or forelimb for further analysis.

TABLE 5
Experimental groups for motor cortex
transplantation iNPC-GDNF^{dox}.

| | | | Injection | |
|---|---|---|---|---|
| | Dose | Dose | concentration | |
| | (cells/animal) | [cells/site] | [cells/μL] | # of Animals |
| CTRL | N/A | N/A | N/A | 10 |
| D1 | 0.4M | 20k | 10,000 | 15 |
| D2 | 1M | 50k | 25,000 | 15 |
| D3 | 2M | 100k | 50,000 | 25* |

*10 animals will not be given dox as a transplantation control

To determine if transplantation of the iNPC-GDNF^{dox} cells are protective of corticospinal motor neurons in layer 5 of the cortex, and possible protection of spinal motor neurons in the cervical, thoracic and lumbar regions of the spinal cord, both the brain and spinal cords can be harvested from each animal. Serial sections of the engraftment regions of interest can be evaluated using IHC. In the cortex, layer 5 pyramidal neurons, indicated by expression of BCL11B or SATB2, can be quantified in regions proximal and distal to engraftments. While SATB2 is not expressed by the target corticospinal motor neuron that is affected in ALS, it can provide information on the relative effects of iNPC-GDNF^{dox} transplants on neighboring cell types in addition to corticospinal motor neurons. ChAT+ motor neurons of the spinal cord can be evaluated as described above.

Dual site transplantations of iNPC-GDNFdox: The final product in patients will need to slow the degeneration of both the cortical and lumbar motor neurons to be the most effective treatment. Therefore, it can be determined if cell delivery into both the spinal cord and motor cortex protects both pools of motor neurons that provides the optimal therapy. In this aim a set of 25 SOD1^{G93A} rats can be transplanted cells at both sites with the most effective dose as determined from each individual site. Ten of these animals will not be given dox to act as a transplantation control. CNS10-NPC-GDNF can then be transplanted at 100K cells/site (middle dose from CNS10-NPC-GDNF IND dose ranging study) into the lumbar spinal cord, and 20K cells/site into the motor cortex in 15 animals as a positive control. To ease the strain on dual transplanted animals, cortical transplantations can occur first with a two-week recovery period prior to lumbar spinal cord transplants. Ten SOD1^{G93A} littermates that did not receive treatment can be evaluated alongside the transplanted animals as a model control. Once recovered from lumbar transplants, all animals can be observed for detailed clinical examinations, including body weight, motor and sensory function of hindlimbs and forelimbs, morbidity and mortality. These observations can be fully blinded. All rats can receive daily immunosuppression as described before, and animals can be sacrificed at disease onset as determined by behavioral qualifications described earlier. Evaluation of brain and spinal cord tissue can be conducted using IHC as described, and the degree of corticospinal motor neuron and spinal motor neuron protection can be scored in conjunction with behavioral observations. Of special interest can be the percent of engraftment, and the protective benefits in this dual site model compared to the individual sites evaluated as discussed above.

iNPC-GDNFdox therapy can result in protection of ChAT+ cells in the spinal cords, and protection of BCL11B+ cells of the motor cortex, in the SOD1G93A rats. If no GDNF expression and/or engraftment of cells is not observed, higher dosing can be used to determine efficacy of dox treatment in wild type rats. If both oral gavage and administration through drinking water proves effective in activating GDNF expression in these animals, administration through drinking water can be used, as this method may best translate to oral delivery in patients. In the event that these constructs are not activatable using the outlined delivery mechanisms, the constitutive product iNPC-GDNF^{CONST} described in Example 1 for efficacy testing in the 3 site models of the SOD1^{G93A} rats can be utilized. In the event that poor survival of engraftments, and/or protection of host neurons are observed, alternative dosing regimens can be achieved with the inducible cells transplanted into wild type rats.

### Example 3: iNPC-GDNFdox Safety in Culture and Long-Term Transplantation in Nude Rats

In this study, the safety and tolerability of iNPC-GDNF^{dox/CONST} in healthy animals without immune suppression is evaluated. A valid concern of iPSC-derived tissue is the danger of run-away proliferation due to the pluripotent stem cell origins. First, the genomic integrity of the cells can be confirmed through whole genome sequencing and the absence of pluripotent cells can be validated using clinically accepted measures. Once an acceptable batch of cells has been produced, the long-term safety and tumorgenicity of the iNPC-GDNF^{dox} therapy will be evaluated in the spinal cord of nude rats. Without being bound by a particular theory it was hypothesized that differentiated, genomically intact cells lacking pluripotency gene expression will prove safe in the immune-compromised rats.

Detection of pluripotency genes and validation of genomic stability: As potential teratoma formation is a concern for iPSC derived products, and in accordance with FDA and ISSCR guidelines, banked lots of iNPC-GDNF^{dox} cells can be validated through a simple qPCR screen for the pluripotency factor OCT-4. This limit of detection assay can assess the number of pluripotency transcripts in differentiated batches of iNPC-GDNF^{dox} compared to iPSCs and has been validated for GMP production of other clinical material. A cell product is considered to have passed if it contains <0.1% of the OCT-4 transcripts detected in the originating iPSCs.

Gene editing techniques and prolonged cell culture manipulations have the potential to generate genomic and karyotypic abnormalities, and so the iNPC-GDNF products must also be verified as stable at a genetic level. The lots which pass detection of OCT-4 will then be submitted for both whole genome sequencing and G-band karyotype analysis. An acceptable score for a lot of iNPC-GDNF^{dox} is defined as one that has been verified to contain the correct GDNF construct in the AAVS1 locus, expresses <0.1% of the OCT-4 transcripts of the originating iPSCs, and has a normal karyotype.

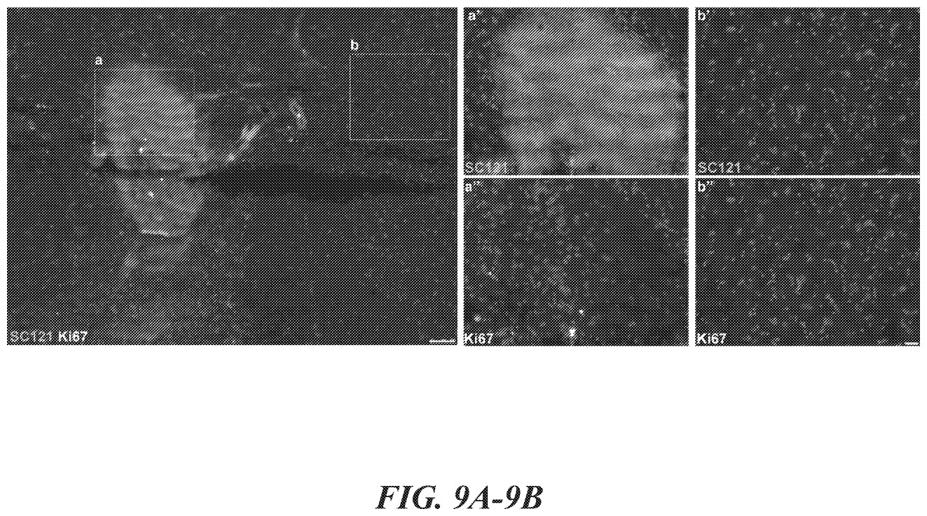

Tumorgenicity and in vivo safety: In accordance with FDA guidelines, healthy animals represent the standard model system employed to conduct traditional toxicology studies^{50,51}. Preliminary studies transplanting 100K cells/site of lentivirally transduced cells from the original iNPC protocol show that these cells engraft efficiently in the nude rat spinal cords and can survive for up to 9 months. Histology for the human nuclei marker and the proliferation marker Ki67 shows that these grafts lose their proliferative behavior over this duration (FIG. 9A-9B). The lack of Ki67 staining suggests that there is little danger of uncontrolled proliferation in iNPC-GDNF transplants.

To evaluate the potential risks associated with long-term transplantation of iNPC-GDNF^{dox}, the toxicology and tumorigenic potential of these cells can be assessed with transplantations into the lumbar spinal cords or motor cortex of immunocompromised, athymic nude rats. 15 animals can be used per group. The highest effective dose in protecting host neurons at each location, as determined in Aim 2, can be used to maximize the chance of forming any undesired effects or responses to engraftment. Animals receiving lumbar spinal cord transplants can receive bilateral injections at a volume of 2 μl per site, at 6 sites spaced 1 mm apart (3 sites/side) along the lumbar spinal cord. Animals receiving transplants into the motor cortex can receive bilateral injections at a volume of 2 μl per site, delivered at the same Bregma locations as listed in Aim 2.2B. Dox administration and dosage, as determined in Aim 2.1, can begin one week post-surgery and continue throughout the duration of the study. All animals can receive dox, as the goal of this aim is to determine the extent of effects caused by both the cells themselves, and secretion of the neurotrophic factor GDNF. Animals from both spinal and cortex studies can be evaluated at 30 days (3 animals/study) and 180 days (12 animals/study) post-transplant. To determine the safety of the iNPC product, animal body weight and gross physiology can be monitored for signs of teratoma formation or impairment of motor activity. At each time point, the respective tissues of engraftment can be collected, sectioned serially, and then assayed using immunohistochemistry. Quantification of transplant survival (detection of human nuclei and cytoplasmic proteins), proliferation (detection of Ki67 co-localized with human markers), host neuron health (detection of neurofilaments, ChAT, BCL11B, SATB2 and TUNEL staining), and host tissue reactivity (upregulation of GFAP expression or other proteins by activated glia). Tumorgenicity analysis on engrafted tissue sections and whole organs can also be performed.

Given the success of other studies using iPSC-derived tissues as safe²⁰ cellular therapeutics, the genomic stability and absence of pluripotent markers can be detected in iNPC-GDNF products. Evaluation of long term engraftment and tumorgenicity of the iNPC-GDNF^{dox} can determine the safety in use as a therapeutic product, and given the amount of engraftment and lack of proliferative cells in the study using the original iNPC-GDNF product, the cells should be proven safe in cortical and spinal transplants. In the event that overt growths are detected, which impair motor behaviors or seem to cause the animals pain, then the next highest dose of cells can be evaluated. The CNS10-NPC-GDNF positive controls can be used in comparison to determine what degree of migration, Ki67 staining, or reactivity is within an acceptable range for therapeutic advancement. Values greater than a 20% difference from observations of the CNS10-NPC-GDNF animals can be considered unacceptable (i.e. 20% more Ki67+ human cells detected). Although unlikely, if the iNPC-GDNF^{dox} express exceedingly high levels of Ki67 expression, an alternative method can be to treat the cells in culture with a γ-secretase inhibitor which reduces proliferation^{52,53}, and these cells could then be dissociated and tested in a small cohort of athymic rats.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the compositions and methods related to induced pluripotent stem cells (iPSCs), differentiated iPSCs including neural progenitor cells, vectors used for manipulation of the aforementioned cells, methods and compositions related to use of the aforementioned compositions, techniques and composition and use of solutions used therein, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventor for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

SEQUENCES

SEQ ID NO: 1-pB-RTP-Tet-GDNF/memClover-FLuc

| 1 | gtcgacttaa ccctagaaag ataatcatat tgtgacgtac gttaaagata atcatgcgta | |
|---|---|---|
| 60 | aaattgacgc atgtgtttta tcgatctgta tatcgaggtt tatttattaa tttgaataga | |
| 120 | tattaagttt tattatattt acacttacat actaataata aattcaacaa acaatttatt | |
| 180 | tatgtttatt tatttattaa aaaaaaacaa aaactcaaaa tttcttctat aaagtaacaa | |
| 240 | aacttttact agttattaat agtaatcaat tacggggtca ttagttcata gcccatatat | |
| 300 | ggagttccgc gttacataac ttacggtaaa tggcccgcct ggctgaccgc ccaacgaccc | |
| 360 | ccgcccattg acgtcaataa tgacgtatgt tcccatagta acgccaatag ggactttcca | |
| 420 | ttgacgtcaa tgggtggagt atttacggta aactgcccac ttggcagtac atcaagtgta | |
| 480 | tcatatgcca agtacgcccc ctattgacgt caatgacggt aaatggcccg cctggcatta | |
| 540 | tgcccagtac atgaccttat gggactttcc tacttggcag tacatctacg tattagtcat | |
| 600 | cgctattacc atgggtcgag gtgagcccca cgttctgctt cactctcccc atctcccccc | |
| 660 | cctccccacc cccaattttg tatttattta ttttttaatt attttgtgca gcgatggggg | |
| 720 | cggggggggg gggggcgcgc gccagggggg gcggggcggg gcgaggggcg gggcggggcg | |
| 780 | aggcggagag gtgcggcggc agccaatcag agcggcgcgc tccgaaagtt tccttttatg | |
| 840 | gcgaggcggc ggcggcggcg gccctataaa aagcgaagcg cgcggcgggc gggagtcgct | |
| 900 | gcgttgcctt cgccccgtgc cccgctccgc gccgcctcgc gccgcccgcc ccggctctga | |
| 960 | ctgaccgcgt tactcccaca ggtgagcggg cgggacggcc cttctcctcc gggctgtaat | |
| 1020 | tagcgcttgg tttaatgacg gctcgtttct tttctgtggc tgcgtgaaag ccttaaaggg | |
| 1080 | ctccgggagg gccctttgtg cgggggggag cggctcgggg ggtgcgtgcg tgtgtgtgtg | |
| 1140 | cgtggggagc gccgcgtgcg gcccgcgctg cccggcggct gtgagcgctg cgggcgcggc | |
| 1200 | gcggggcttt gtgcgctccg cgtgtgcgcg aggggagcgc ggccgggggc ggtgccccgc | |
| 1260 | ggtgcggggg ggctgcgagg ggaacaaagg ctgcgtgcgg ggtgtgtgcg tgggggggtg | |
| 1320 | agcagggggt gtgggcgcgg cggtcgggct gtaacccccc cctgcacccc cctccccgag | |
| 1380 | ttgctgagca cggcccggct tcgggtgcgg ggctccgtgc ggggcgtggc gcggggctcg | |
| 1440 | ccgtgccggg cggggggtgg cggcaggtgg gggtgccggg cggggcgggg ccgcctcggg | |
| 1500 | ccggggaggg ctcgggggag gggcgcggcg gcccccggag cgccggcggc tgtcgaggcg | |
| 1560 | cggcgagccg cagccattgc cttttatggt aatcgtgcga gagggcgcag ggacttcctt | |
| 1620 | tgtcccaaat ctgtgcggag ccgaaatctg ggaggcgccg ccgcaccccc tctagcgggc | |
| 1680 | gcggggcgaa gcggtgcggc gccggcagga aggaaatggg cggggagggc cttcgtgcgt | |
| 1740 | cgccgcgccg ccgtcccctt ctccctctcc agcctcgggg ctgtccgcgg ggggacggct | |
| 1800 | gccttcgggg gggacggggc agggcggggt tcggcttctg gcgtgtgacc ggcggctcta | |
| 1860 | gagcctctgc taaccatgtt catgccttct tctttttcct acagctcctg ggcaacgtgc | |
| 1920 | tggttattgt gctgtctcat cattttggca aagaattcgg taccgcatgc atcgatgcta | |
| 1980 | gcgcgccgcc accatgtcta gactggacaa gagcaaagtc ataaactctg ctctggaatt | |
| 2040 | actcaatgga gtcggtatcg aaggcctgac gacaaggaaa ctcgctcaaa agctgggagt | |
| 2100 | tgagcagcct accctgtact ggcacgtgaa gaacaagcgg gccctgctcg atgccctgcc | |
| 2160 | aatcgagatg ctggacaggc atcataccca ctcctgcccc ctggaaggcg agtcatggca | |
| 2220 | agactttctg cggaacaacg ccaagtcata ccgctgtgct ctcctctcac atcgcgacgg | |
| 2280 | ggctaaagtg catctcggca cccgcccaac agagaaacag tacgaaaccc tggaaaatca | |
| 2340 | gctcgcgttc ctgtgtcagc aaggcttctc cctggagaac gcactgtacg ctctgtccgc | |
| 2400 | cgtgggccac tttacactgg gctgcgtatt ggaggaacag gagcatcaag tagcaaaaga | |
| 2460 | ggaaagagag acacctacca ccgattctat gcccccactt ctgaaacaag caattgagct | |
| 2520 | gttcgaccgg cagggagccg aacctgcctt ccttttcggc ctggaactaa tcatatgtgg | |
| 2580 | cctggagaaa cagctaaagt gcgaaagcgg cgggccgacc gacgcccttg acgattttga | |
| 2640 | cttagacatg ctcccagccg atgcccttga cgactttgac cttgatatgc tgcctgctga | |
| 2700 | cgctcttgac gattttgacc ttgacatgct ccccggggga tccggaggat ccggagccac | |
| 2760 | gaacttctct ctgttaaagc aagcaggaga cgtggaagaa aaccccggtc ctatgagcga | |
| 2820 | gctgattaag gagaacatgc acatgaagct gtacatggag ggcaccgtgg acaaccatca | |
| 2880 | cttcaagtgc acatccgagg gcgaaggcaa gccctacgag ggcacccaga ccatgagaat | |
| 2940 | caaggtggtc gagggcggcc ctctcccctt cgccttcgac atcctggcta ctagcttcct | |
| 3000 | ctacggcagc aagaccttca tcaaccacac ccagggcatc cccgacttct tcaagcagtc | |
| 3060 | cttccctgag ggcttcacat gggagagagt caccacatac gaagacgggg gcgtgctgac | |
| 3120 | cgctacccag gacaccagcc tccaggacgg ctgcctcatc tacaacgtca agatcagagg | |
| 3180 | ggtgaacttc acatccaacg gccctgtgat gcagaagaaa acactcggct gggaggcctt | |
| 3240 | caccgagacg ctgtaccccg ctgacggcgg cctggaaggc agaaacgaca tggccctgaa | |
| 3300 | gctcgtgggc gggagccatc tgatcgcaaa cgccaagacc acatatagat ccaagaaacc | |
| 3360 | cgctaagaac ctcaagatgc ctggcgtcta ctatgtggac tacagactgg aaagaatcaa | |
| 3420 | ggaggccaac aacgagacct acgtcgagca gcacgaggtg gcagtggcca gatactgcga | |
| 3480 | cctccctagc aaactggggc acaagcttaa ttccggactc ggcaagccta tccctaaccc | |
| 3540 | tctgctgggc ctggacagca ccgatccaaa aaagaagaga aaggtagacc ctaagaagaa | |
| 3600 | gaggaaggtg gaccccaaga agaagagaaa ggtgtgaggc agtggaggga gtggagcaac | |
| 3660 | caatttttca ctcctgaagc aagctgggga tgtagaggag aatcctgggc ctatggacta | |
| 3720 | caaagacgat gacgacaagc ttggcactag tggctttgcg aatgaattgg gacctaggtt | |
| 3780 | gatgggcaag ctttctagtc aactcgagat gaccgagtac aagcccacgg tgcgcctcgc | |
| 3840 | cacccgcgac gacgtcccca gggccgtacg caccctcgcc gccgcgttcg ccgactaccc | |
| 3900 | cgccacgcgc cacaccgtcg atccggaccg ccacatcgag cgggtcaccg agctgcaaga | |
| 3960 | actcttcctc acgcgcgtcg ggctcgacat cggcaaggtg tgggtcgcgg acgacggcgc | |
| 4020 | cgcggtggcg gtctggacca cgccggagag cgtcgaagcg ggggcggtgt tcgccgagat | |
| 4080 | cggcccgcgc atggccgagt tgagcggttc ccggctggcc gcgcagcaac agatggaagg | |
| 4140 | cctcctggcg ccgcaccggc ccaaggagcc cgcgtggttc ctggccaccg tcggcgtctc | |
| 4200 | gcccgaccac cagggcaagg gtctgggcag cgccgtcgtg ctccccggag tggaggcggc | |
| 4260 | cgagcgcgcc ggggtgcccg ccttcctgga gacctccgcg ccccgcaacc tccccttcta | |
| 4320 | cgagcggctc ggcttcaccg tcaccgccga cgtcgaggtg cccgaaggac cgcgcacctg | |
| 4380 | gtgcatgacc cgcaagcccg gtgcctgagc ggccgcactc ctcaggtgca ggctgcctat | |
| 4440 | cagaaggtgg tggctggtgt ggccaatgcc ctggctcaca aataccactg agatcttttt | |
| 4500 | ccctctgcca aaaattatgg ggacatcatg aagccccttg agcatctgac ttctggctaa | |
| 4560 | taaaggaaat ttattttcat tgcaatagtg tgttggaatt ttttgtgtct ctcactcgga | |
| 4620 | aggacatatg ggagggcaaa tcatttaaaa catcagaatg agtatttggt ttagagtttg | |
| 4680 | gcaacatatg cccatatgct ggctgccatg aacaaaggtt ggctataaag aggtcatcag | |
| 4740 | tatatgaaac agccccctgc tgtccattcc ttattccata gaaaagcctt gacttgaggt | |
| 4800 | tagatttttt ttatattttg ttttgtgtta tttttttctt taacatccct aaaattttcc | |
| 4860 | ttacatgttt tactagccag atttttcctc ctctcctgac tactcccagt catagctgtc | |
| 4920 | cctcttctct tatggagatc cctcgacctg ggtaacgcca gggttttccc agtcacgacg | |
| 4980 | ttgtaaaacg acggccagtg ccaagcttgc atgcctgcag taagatacat tgatgagttt | |
| 5040 | ggacaaacca caactagaat gcagtgaaaa aaatgcttta tttgtgaaat ttgtgatgct | |
| 5100 | attgctttat ttgtaaccat tataagctgc aataaacaag ttaacaacaa caattgcatt | |
| 5160 | cattttatgt ttcaggttca gggggaggtg tgggaggttt tttaaagcaa gtaaaacctc | |
| 5220 | tacaaatgtg gtaggtacca ccggtcctgc aggttagacg ttgatcctgg cgctggcgca | |
| 5280 | agcagcaggg tctctatcca tgccgctctc ctgggcgcag ctcatgggca gggtgccggc | |
| 5340 | ggcctgctcc tccacctcgg gagggaagcc gtgagaattc acggcgatct tgccgccctt | |
| 5400 | cttggcctta atgagaatct cgcggatctt gcgggcgtcc aacttgccgg tcagtccttt | |
| 5460 | aggcacctcg tccacgaaca caacaccacc gcgcagcttc ttggcggttg taacctggct | |
| 5520 | ggccacatag tccacgatct ccttctcggt catggtttta ccgtgttcca gcacgacgac | |
| 5580 | tgcggcgggc agctcgccgg catcgtcgtc gggcaggccg gcgaccccgg cgtcgaagat | |
| 5640 | gttggggtgt tgcagcagga tgctctccag ttcggctggg gctacctggt agcccttgta | |
| 5700 | tttgatcagg ctcttcagcc ggtccacgat gaagaagtgc tcgtcctcgt cccagtaggc | |
| 5760 | gatgtcgccg ctgtgcagcc agccgtcctt gtcgatgaga gcgtttgtag cctcggggtt | |
| 5820 | gttaacgtag ccgctcatga tcatggggcc acggacgcac agctcgccgc gctggttcac | |
| 5880 | acccagtgtc ttaccggtgt ccaagtccac caccttagcc tcgaagaagg gcaccacctt | |
| 5940 | gcctactgcg ccaggcttgt cgtccccttc gggggtgatc agaatggcgc tggttgtttc | |
| 6000 | tgtcaggccg tagccctggc ggatgcctgg taggtggaag cgtttggcca cggcctcacc | |
| 6060 | tacctccttg ctgagcggcg ccccgccgct ggcgatctcg tgcaagttgc ttaggtcgta | |
| 6120 | cttgtcgatg agagtgctct tagcgaagaa gctaaatagt gtgggcacca gcagggcaga | |
| 6180 | ttgaatctta tagtcttgca agctgcgcaa gaatagctcc tcctcgaagc ggtacatgag | |
| 6240 | cacgacccga aagccgcaga tcaagtagcc cagcgtggtg aacatgccga agccgtggtg | |
| 6300 | aaatggcacc acgctgagga tagcggtgtc ggggatgatc tggttgccga agatggggtc | |
| 6360 | gcgggcatga ctgaatcgga cacaagcggt gcggtgcggt agggctacgc ccttgggcaa | |
| 6420 | tccggtactg ccactactgt tcatgatcag ggcgatggtt ttgtcccggt cgaagctctc | |
| 6480 | gggcacgaag tcgtactcgt tgaagccggg tggcaaatgg gaagtcacga aggtgtacat | |
| 6540 | gctttggaag ccctggtagt cggtcttgct atccatgatg atgatctttt gtatgatcgg | |
| 6600 | tagcttcttt tgcacgttga ggatcttttg cagccctttc ttgctcacga atacgacggt | |
| 6660 | gggctggctg atgcccatgc tgttcagcag ctcgcgctcg ttgtagatgt cgttagctgg | |
| 6720 | ggccacagcc acaccgatga acagggcacc caacacgggc atgaagaact gcaagctatt | |
| 6780 | ctcgctgcac accacgatcc gatggtttgt attcagccca tagcgcttca tagcttctgc | |
| 6840 | cagccgaacg ctcatctcga agtactcggc gtaggtaatg tccacctcga tatgtgcgtc | |
| 6900 | ggtaaaggcg atggtgccgg gcaccagggc gtagcgcttc atggctttgt gcagctgctc | |
| 6960 | gccggcggtc ccgtcttcga gtgggtagaa tggcgctggg cccttcttaa tgtttttggc | |
| 7020 | atcttccata ggaccggggt tttcttccac gtctcctgct tgctttaaca gagagaagtt | |
| 7080 | cgtggctccg gatccggcgg cggttacgaa ctccagcagg accatgtgat cgcgcttctc | |
| 7140 | gttggggtct ttgctcaggg cggactgatg gctcaggtag tcgttgtcgg gcagcagcac | |
| 7200 | ggggccgtcg ccgatggggg tgttctgctg gtagtggtcg gcgagctgca cgctgccgtc | |
| 7260 | ctcaacgttg tggcggatct tgaagttagc cttgatgccg ttcttctgct tgtcggccgt | |
| 7320 | gatatagacg ttgtggctgt tgaagttgta ctccagcttg tgccccagga tcttgccgtc | |
| 7380 | ctccttgaag tcgatgccct tcagctcgat gcggttcacc agggtgtcgc cctcgaactt | |
| 7440 | cacctcggcg cgggtcttgt aggtaccgtc gtccttgaaa gagatggtgc gctcctggac | |
| 7500 | gtagccttcg ggcatggcgg acttgaagaa gtcgtgctgc ttcatgtggt cggggtagcg | |
| 7560 | gctgaagcag gccacgccgt agccgaaggt ggtcacgagg gtgggccagg gcacgggcag | |
| 7620 | cttgccggtg gtgcagatga acttcagggt cagcttgccg ttggtggcat cgccctcgcc | |
| 7680 | ctcgccgcgg acgctgaact tgtggccgtt tacgtcgccg tccagctcga ccaggatggg | |
| 7740 | caccaccccg gtgaacagct cctcgccctt gctcaccatg tccacgccgt cgtcgttcag | |
| 7800 | gttgtccttg cgcttgctct tgatgcagcc catggtggcg gcggtcacca cgcgtccgcg | |
| 7860 | atctgacggt tcactaaacg agctctgctt atataggcct cccaccgtac acgccacctc | |
| 7920 | gacatactcg agtttactcc ctatcagtga tagagaacgt atgaagagtt tactccctat | |
| 7980 | cagtgataga gaacgtatgc agactttact ccctatcagt gatagagaac gtataaggag | |
| 8040 | tttactccct atcagtgata gagaacgtat gaccagttta ctccctatca gtgatagaga | |
| 8100 | acgtatctac agtttactcc ctatcagtga tagagaacgt atatccagtt tactccctat | |
| 8160 | cagtgataga gaacgtataa gctttaggcg tgtacggtgg gcgcctataa aagcagagct | |
| 8220 | cgtttagtga accgtcagat cgcctggagc aattccacaa cacttttgtc ttataccaac | |
| 8280 | tttccgtacc acttcctacc ctcgtaaaca gctggccgcc accatgaagt tatgggatgt | |
| 8340 | cgtggctgtc tgcctggtgc tgctccacac cgcgtccgcc ttcccgctgc ccgccggtaa | |
| 8400 | gaggcctccc gaggcgcccg ccgaagaccg ctccctcggc cgccgccgcg cgcccttcgc | |
| 8460 | gctgagcagt gactcaaata tgccagagga ttatcctgat cagttcgatg atgtcatgga | |
| 8520 | ttttattcaa gccaccatta aaagactgaa aaggtcacca gataaacaaa tggcagtgct | |
| 8580 | tcctagaaga gagcggaatc ggcaggctgc agctgccaac ccagagaatt ccagaggaaa | |
| 8640 | aggtcggaga ggccagaggg gcaaaaaccg gggttgtgtc ttaactgcaa tacatttaaa | |
| 8700 | tgtcactgac ttgggtctgg gctatgaaac caaggaggaa ctgattttta ggtactgcag | |
| 8760 | cggctcttgc gatgcagctg agacaacgta cgacaaaata ttgaaaaact tatccagaaa | |
| 8820 | tagaaggctg gtgagtgaca aagtagggca ggcatgttgc agacccatcg cctttgatga | |
| 8880 | tgacctgtcg tttttagatg ataacctggt ttaccatatt ctaagaaagc attccgctaa | |
| 8940 | aaggtgtgga tgtatctgat tcgaaagccg cactcctcat aatcaacctc tggattacaa | |
| 9000 | aatttgtgaa agattgactg gtattcttaa ctatgttgct ccttttacgc tatgtggata | |
| 9060 | cgctgcttta atgcctttgt atcatgctat tgcttcccgt atggctttca ttttctcctc | |
| 9120 | cttgtataaa tcctggttgc tgtctcttta tgaggagttg tggcccgttg tcaggcaacg | |
| 9180 | tggcgtggtg tgcactgtgt ttgctgacgc aacccccact ggttggggca ttgccaccac | |
| 9240 | ctgtcagctc ctttccggga ctttcgcttt ccccctccct attgccacgg cggaactcat | |
| 9300 | cgccgcctgc cttgcccgct gctggacagg ggctcggctg ttgggcactg acaattccgt | |
| 9360 | ggtgttgtcg gggaaatcat cgtcctttcc ttggctgctc gcctgtgttg ccacctggat | |
| 9420 | tctgcgcggg acgtccttct gctacgtccc ttcggccctc aatccagcgg accttccttc | |
| 9480 | ccgcggcctg ctgccgggct tgcggcctct tccgcgtctt cgccttcgcc ctcagacgag | |
| 9540 | tcggatctcc ctttgggccg cctccccgca ggccgcacac ctcaggtgca ggctcgaaat | |
| 9600 | ttaaatcctc gagggccgca ctcctcaggt gcaggctgcc tatcagaagg tggtggctgg | |
| 9660 | tgtggccaat gccctggctc acaaatacca ctgagatctt tttccctctg ccaaaaatta | |
| 9720 | tggggacatc atgaagcccc ttgagcatct gacttctggc taataaagga aatttatttt | |
| 9780 | cattgcaata gtgtgttgga attttttgtg tctctcactc ggaaggacat atgggagggc | |
| 9840 | aaatcattta aaacatcaga atgagtattt ggtttagagt ttggcaacat atgccatatg | |
| 9900 | ctggctgcca tgaacaaagg tggctataaa gaggtcatca gtatatgaaa cagccccctg | |
| 9960 | ctgtccattc cttattccat agaaaagcct tgacttgagg ttagattttt tttatatttt | |
| 10020 | gttttgtgtt atttttttct ttaacatccc taaaattttc cttacatgtt ttactagcca | |
| 10080 | gatttttcct cctctcctga ctactcccag tcatagctgt ccctcttctc ctgcagatat | |
| 10140 | ctataacaag aaaatatata tataataagt tatcacgtaa gtagaacatg aaataacaat | |
| 10200 | ataattatcg tatgagttaa atcttaaaag tcacgtaaaa gataatcatg cgtcattttg | |
| 10260 | actcacgcgg tcgttatagt tcaaaatcag tgacacttac cgcattgaca agcacgcctc | |
| 10320 | acgggagctc caagcggcga ctgagatgtc ctaaatgcac agcgacggat tcgcgctatt | |
| 10380 | tagaaagaga gagcaatatt tcaagaatgc atgcgtcaat tttacgcaga ctatctttct | |
| 10440 | agggttaagc ttggcgtaat catggtcata gctgtttcct gtgtgaaatt gttatccgct | |
| 10500 | cacaattcca cacaacatac gagccggaag cataaagtgt aaagcctggg gtgcctaatg | |
| 10560 | agtgagctaa ctcacattaa ttgcgttgcg ctcactgccc gctttccagt cgggaaacct | |
| 10620 | gtcgtgccag cggatccgca tctcaattag tcagcaacca tagtcccgcc cctaactccg | |
| 10680 | cccatcccgc ccctaactcc gcccagttcc gcccattctc cgccccatgg ctgactaatt | |
| 10740 | ttttttattt atgcagaggc cgaggccgcc tcggcctctg agctattcca gaagtagtga | |
| 10800 | ggaggctttt ttggaggcct aggcttttgc aaaaagctaa cttgtttatt gcagcttata | |
| 10860 | atggttacaa ataaagcaat agcatcacaa atttcacaaa taaagcattt ttttcactgc | |
| 10920 | attctagttg tggtttgtcc aaactcatca atgtatctta tcatgtctgg atccgctgca | |
| 10980 | ttaatgaatc ggccaacgcg cggggagagg cggtttgcgt attgggcgct cttccgcttc | |
| 11040 | ctcgctcact gactcgctgc gctcggtcgt tcggctgcgg cgagcggtat cagctcactc | |
| 11100 | aaaggcggta atacggttat ccacagaatc aggggataac gcaggaaaga acatgtgagc | |
| 11160 | aaaaggccag caaaaggcca ggaaccgtaa aaaggccgcg ttgctggcgt ttttccatag | |
| 11220 | gctccgcccc cctgacgagc atcacaaaaa tcgacgctca agtcagaggt ggcgaaaccc | |
| 11280 | gacaggacta taaagatacc aggcgtttcc ccctggaagc tccctcgtgc gctctcctgt | |
| 11340 | tccgaccctg ccgcttaccg gatacctgtc cgcctttctc ccttcgggaa gcgtggcgct | |
| 11400 | ttctcaatgc tcacgctgta ggtatctcag ttcggtgtag gtcgttcgct ccaagctggg | |
| 11460 | ctgtgtgcac gaaccccccg ttcagcccga ccgctgcgcc ttatccggta actatcgtct | |
| 11520 | tgagtccaac ccggtaagac acgacttatc gccactggca gcagccactg gtaacaggat | |
| 11580 | tagcagagcg aggtatgtag gcggtgctac agagttcttg aagtggtggc ctaactacgg | |
| 11640 | ctacactaga aggacagtat ttggtatctg cgctctgctg aagccagtta ccttcggaaa | |
| 11700 | aagagttggt agctcttgat ccggcaaaca aaccaccgct ggtagcggtg gtttttttgt | |
| 11760 | ttgcaagcag cagattacgc gcagaaaaaa aggatctcaa gaagatcctt tgatcttttc | |
| 11820 | tacggggtct gacgctcagt ggaacgaaaa ctcacgttaa gggattttgg tcatgagatt | |
| 11880 | atcaaaaagg atcttcacct agatcctttt aaattaaaaa tgaagtttta aatcaatcta | |
| 11940 | aagtatatat gagtaaactt ggtctgacag ttaccaatgc ttaatcagtg aggcacctat | |
| 12000 | ctcagcgatc tgtctatttc gttcatccat agttgcctga ctccccgtcg tgtagataac | |
| 12060 | tacgatacgg gagggcttac catctggccc cagtgctgca atgataccgc gagacccacg | |
| 12120 | ctcaccggct ccagatttat cagcaataaa ccagccagcc ggaagggccg agcgcagaag | |
| 12180 | tggtcctgca actttatccg cctccatcca gtctattaat tgttgccggg aagctagagt | |
| 12240 | aagtagttcg ccagttaata gtttgcgcaa cgttgttgcc attgctacag gcatcgtggt | |
| 12300 | gtcacgctcg tcgtttggta tggcttcatt cagctccggt tcccaacgat caaggcgagt | |
| 12360 | tacatgatcc cccatgttgt gcaaaaaagc ggttagctcc ttcggtcctc cgatcgttgt | |
| 12420 | cagaagtaag ttggccgcag ttttatcact catggttatg gcagcactgc ataattctct | |
| 12480 | tactgtcatg ccatccgtaa gatgcttttc tgtgactggt gagtactcaa ccaagtcatt | |
| 12540 | ctgagaatag tgtatgcggc gaccgagttg ctcttgcccg gcgtcaatac gggataatac | |
| 12600 | cgcgccacat agcagaactt taaaagtgct catcattgga aaacgttctt cggggcgaaa | |
| 12660 | actctcaagg atcttaccgc tgttgagatc cagttcgatg taacccactc gtgcacccaa | |
| 12720 | ctgatcttca gcatctttta ctttcaccag cgtttctggg tgagcaaaaa caggaaggca | |
| 12780 | aaatgccgca aaaaagggaa taagggcgac acggaaatgt tgaatactca tactcttcct | |
| 12840 | ttttcaatat tattgaagca tttatcaggg ttattgtctc atgagcggat acatatttga | |
| 12900 | atgtatttag aaaaataaac aaataggggt tccgcgcaca tttccccgaa aagtgccacc | |
| 12960 | tg | 12962 |

SEQ ID NO: 2-GDNF gene reference sequence
NCBI Reference sequence: NC_000005.10:c37840044-37812677
SEQ ID NO: 3-GDNF mRNA transcript sequence
NCBI Reference sequence: NM_000514.4
Homo sapiens glial cell derived neurotrophic factor (GDNF), transcript variant 1,
mRNA
AGCCTGCGCTCCTGGCGCCCTCATGTCTTCACGGGACTCCCCGCGCCGGTTGACGTGGTGTCTCGTTCGG
ATCTCCAGGCAAGACCTCAGCTCCGGCAGCAGCATCAGACAAACCAGTCTCGTGCTCCCAGGCAGTGCGC
CCAGAGGAGGCGCAGAGCGCGGCAGCTGCCGCTGAGCCGCCCGCAGCGCCCCGGGCCCGCGCAGCCCCAG
CCAAGAGCGCGACGCGCGCAGCCCTGTCAGCCCCCCACCCAAAGCAGCGGCGGCTGCTCGGACCTCGGCT
TCTGGGGGTGCGGGGGCCCGGCGGGAGAGTTGCCGGCAGCCCTCGCCCTGTTGGCGGCGGCGGCGGCGGG
AGTCTTGGCCGCCGCCTCCAGCGCGCCCTTGCTGCCCCGCGCGACCCCAGGATTGCGAACTCTTGCCCCT
GACCTGTTGGGGGGGGCTCCGCGCTCCAGCCATCAGCCCGGATGGGTCTCCTGGCTGGGACTTGGGGCAC
CTGGAGTTAATGTCCAACCTAGGGTCTGCGGAGACCCGATCCGAGGTGCCGCCGCCGGACGGGACTTTAA
GATGAAGTTATGGGATGTCGTGGCTGTCTGCCTGGTGCTGCTCCACACCGCGTCCGCCTTCCCGCTGCCC
GCCGGTAAGAGGCCTCCCGAGGCGCCCGCCGAAGACCGCTCCCTCGGCCGCCGCCGCGCGCCCTTCGCGC
TGAGCAGTGACTCAAATATGCCAGAGGATTATCCTGATCAGTTCGATGATGTCATGGATTTTATTCAAGC
CACCATTAAAAGACTGAAAAGGTCACCAGATAAACAAATGGCAGTGCTTCCTAGAAGAGAGCGGAATCGG
CAGGCTGCAGCTGCCAACCCAGAGAATTCCAGAGGAAAAGGTCGGAGAGGCCAGAGGGGCAAAAACCGGG
GTTGTGTCTTAACTGCAATACATTTAAATGTCACTGACTTGGGTCTGGGCTATGAAACCAAGGAGGAACT
GATTTTTAGGTACTGCAGCGGCTCTTGCGATGCAGCTGAGACAACGTACGACAAAATATTGAAAAACTTA
TCCAGAAATAGAAGGCTGGTGAGTGACAAAGTAGGGCAGGCATGTTGCAGACCCATCGCCTTTGATGATG
ACCTGTCGTTTTTAGATGATAACCTGGTTTACCATATTCTAAGAAAGCATTCCGCTAAAAGGTGTGGATG
TATCTGACTCCGGCTCCAGAGACTGCTGTGTATTGCATTCCTGCTACAGTGCAAAGAAAGGGACCAAGGT
TCCCAGGAAATGTTTGCCCAGAATGGAAGATGAGGACCAAGGAGGCGGAGGAGGAGGAAGAAGAAGAGGA
GGAGGAGGAGGAGGAGGAGGAGGAGGAGGAAGGCAGCCATCATGGGAGCCTGGTAGAGGGAGATCCAGCT
ACAGACAACTGGACAGGAGAGAGAGAAAACAGCCCTCTGGATTCTCCAGGATGGCAGCCGATGTCACTAG
AAGCTCAGGGCTGATGTTCCTGGTTGGCTATTGCCACCATTTCAGCTGATACAGTCCACCATCACTGATT
ACCGGCGCGGTTGCGGTGGATGCACTTGAACCAAACCAGTGTATCTCCTGTGATTTGTTTTCATGTGTCC
GAAGACACCAGGGAAACAGAGATCCTGGTGTTGTTCCTTGTTATTACGTTTTACTGCTGAAAGTAAGAGG
TTTATTTTTCTGTCACTCAGTGGAGACATACCCTGGAAAGGAGAGGGGAAAAAAAAAGCAAAGATACAAG
AGATAATCACCTTTGCATTTGAAAGTTGAGGCCCGAGGTTTACTACAACCAGCATTTTTGCCAACGGTTG
GTGATTGATTTCCATCACGGTGTGTGGGGTGGGAAGAAGTTGGCTAGGAACCAAAAAGGCTGTGCTCATG
ATTAAACACAAACCTGAAGGTATTTCCTTTATGTCCTTGGAAACAGGAAACGAGTTGTGGTTTTCGCCAG
CATTCTTGTAGGAGAGAATCGGGGAAGGCCCCGAACTGCCCCCGGGCAGGGAGAGCCCCTCAGGCCTGTT
GGTTTACAGAGAGACAGATGTTACATAACCAGCTCCGTTGATGCGTGGTCACCAGTGACCAGAGAAGCTA
CTCGATGCAATGCATCTGTTTCAGATACAGAAATATAGAGAAGATATTTATTGAAATTTAAGTTATTGTT
ATTTATTACCGTTCACTAATGAATTTCTCTTTTTTCCCTTATTTATTAAAGTTTCTTTTCAAAGGTGCCA
AAGTATATGTGCTCGCAAAATGCAAAGAAAGGTGACAAAAGGAAATTTGAATTGGGAACAAGGGTCCATG
CTTTTCAAAGTATTAAAAAGTTTTTTGCCAGGCAAAAATCACTTACTTTACCTTTTTAAGAAAATTTGTC
ATTAATTTTCCCCAGATTTCAGCATTTTTCCCAATTTTTATTTGTGGAGCATCTCAGGCAAGCCCCCTTT
CCTGGAGCAGCGTGCAGAGACCACTGGCACTTGACTTTATTTCTTCCTTGCTCCATTGCTGAACAGAAAT
GTCGTGGGCTCCACTTCCTGTTGTCTTTAAGCTCTTAGTCCCCTCCACGTATACCTATCTGTACTATGCA
TAACCATATGTAGAAAAGGTTCAGTTCCTTTTAGTAGGTAGTCCTGGATTTAATGCTGACCTAAAAGTAA
TGTCGACAATGCTGTCAGGTAGCTGCCGTTCTACCGACTCCCTCCATCCCTGCCCACCCACTGCCCTCCC
GAGAATATGCTGGCTGCCCAGTGCAGCCCGGGAGACACAGGGGCCTTCCAGAGGTAGGGTCTACCAGGTC
CTGTACAACCCCTGGGCTGTCACCGGGGGTCAACAGCTGCTGCTCCTATATACCCAAACACCTGACAGCT
CCCTGGGGAGCAGATGGCTGAGAAGGGTGCTGAGGAAGCCATATTGGGACCAGCCACAGCCACACACATG
GAGCCTCATACTTAGGAGCGTGCTGCCTTTAAATGAAGGTGGTCGGGGCCAGTGCAGCGGCTCACACCCA
TAATCCCAACACTTTGGAAAGCCAAGGTGGGAGGATCTCTTGAACCCAGGAGTTTGAGACCAGCTTGGGC
AACATAGGGAGACCCTGTCTCTACAGAAACTTTAAAAATTAGGCAGGCATGATGGTGCACACCTGTGGTC
CCAGCTACTCAAGAGGCTGAAGGAGGATCACTTGAGTCCAGAAGGTCGAGGCTGCAGTGAGCTGTGATCA
TGCCACTGCACTCCAGCCTAAGTGACAGTGCGGTACCCTGTCTCAAAAAAAAAAAAAAAAAAAAAAAGAG
GTTGGAGCAGGAGGAAGCATAGGGGGGGGAACAGCCACCTCCTCCATGCCCTAGATTGTGAATTTATCGG
GCAGCCAACACATGTATGACACACTAGGCCCTGTATTACAGCTTGTTACGCATTTCATAAAAGGGATTTT
CATTAAGGAGATAATCTATTACTACCTACCTTAGTGGCTACTAGTATAAAACTATGACAGATTTAGCAAT
TAAATGAAATACTGGCCTCCATCAAATAATCATAGTAACAAGAAGCAGCAGTTACCAGACATCTGATCCC
CTTCCCCCAAAATACCCAAATTCTTCATGGTTCTGCCCTTCTCTGTCCTTTCTGCTCCCCTTGCTCGCCT
GGGAAATGGAGGAAAGGCCTTCCCTCTCACACTGTCTTGGGATCTTGCTGAGAATTCAGACTGCTCGAAA
CAGTGACAAACCCCAGCCATCCAGTCATTCGTGGAGCACAATTTGGATGTGGCCCCAGGGGCATCTGTCC
CATTCAGAGAACCTTGGCAGTGCGATGGCCACTGTTCCCAGGCTTCAACCTCAGTGACCCCCCCCAACAA
CTCCCCATGGAGAGTCCCTGCCCAAAAAAGCTGTAGGATCCAAGGGGTGTCAATAGCTCGTTCCCGGCAT
CACCTACACACCACAAGCAGGTTTTAATGGAAGCAAGTTGCTCCACCAAATCCACAAAAGGGTAAAGTTT
GTGATTTTTCTTTATCATTGCGATCACCATCTGATACCGTAAGGAGTGCACTTGTTTGGAAGTTCTGACT
TCTCTGATCTGTCTTGGTCGTTTGTGTTATAAAACCAAAGTTCTCTACAGACTTTATTTTTGTACAATAT
CATTTTGTAACTTTTTACAAATAAAAACTCATTTCTATTGC (SEQ ID NO: 3)
SEQ ID NO: 4-GDNF amino acid sequence
NCBI Reference Sequence: NP_000505.1
glial cell line-derived neurotrophic factor isoform 1 preproprotein [Homo sapiens]
MKLWDVVAVCLVLLHTASAFPLPAGKRPPEAPAEDRSLGRRRAPFALSSDSNMPEDYPDQFDDVMDFIQA
TIKRLKRSPDKOMAVLPRRERNROAAAANPENSRGKGRRGORGKNRGCVLTAIHLNVTDLGLGYETKEEL
IFRYCSGSCDAAETTYDKILKNLSRNRRLVSDKVGQACCRPIAFDDDLSFLDDNLVYHILRKHSAKRCGC
I (SEQ ID NO: 4)
SEQ ID NO: 5-AAVS1-Tet-On-3G-GDNF
cagtactaagctttactagggacaggattggtgacagaaaagccccatccttaggcctcctccttccta
gtctcctgatattgggtctaacccccacctcctgttaggcagattccttatctggtgacacacccccat
ttcctggagccatctctctccttgccagaacctctaaggtttgcttacgatggagccagagaggatcct
gggagggagagcttggcagggggtgggagggaagggggggatgcgtgacctgcccggttctcagtggcc
accctgcgctaccctctcccagaacctgagctgctctgacgcggctgtctggtgcgtttcactgatcct
ggtgctgcagcttccttacacttcccaagaggagaagcagtttggaaaaacaaaatcagaataagttgg
tcctgagttctaactttggctcttcacctttctagtccccaatttatattgttcctccgtgcgtcagtt
ttacctgtgagataaggccagtagccagccccgtcctggcagggctgtggtgaggaggggggtgtccgt
gtggaaaactccctttgtgagaatggtgcgtcctaggtgttcaccaggtcgtggccgcctctactccct
ttctctttctccatccttctttccttaaagagtccccagtgctatctgggacatattcctccgcccaga
gcagggtcccgcttccctaaggccctgctctgggcttctgggtttgagtccttggcaagcccaggagag
gcgctcaggcttccctgtcccccttcctcgtccaccatctcatgcccctggctctcctgccccttccct
acaggggttcctggctctgctctaagggcaagggcgaattcgcggccgctaaattcaattcgccctata
gtgagtcgtattacaattcactggccgtcgttttacaacgtcgtgactgggaaaaccctggcgttaccc
aacttaatcgccttgcagcacatccccctttcgccagctggcgtaatagcgaagaggcccgcaccgatc
gcccttcccaacagttgcgcagcctatacgtacggcagtttaaggtttacacctataaaagagagagcc
gttatcgtctgtttgtggatgtacagagtgatattattgacacgccggggcgacggatggtgatccccc
tggccagtgcacgtctgctgtcagataaagtctcccgtgaactttacccggtggtgcatatcggggatg
aaagctggcgcatgatgaccaccgatatggccagtgtgccggtctccgttatcggggaagaagtggctg
atctcagccaccgcgaaaatgacatcaaaaacgccattaacctgatgttctggggaatataaatgtcag
gcatgagattatcaaaaaggatcttcacctagatccttttcacgtagaaagccagtccgcagaaacggt
gctgaccccggatgaatgtcagctactgggctatctggacaagggaaaacgcaagcgcaaagagaaagc
aggtagcttgcagtgggcttacatggcgatagctagactgggcggttttatggacagcaagcgaaccgg
aattgccagctggggcgccctctggtaaggttgggaagccctgcaaagtaaactggatggctttcttgc
cgccaaggatctgatggcgcaggggatcaagctctgatcaagagacaggatgaggatcgtttcgcatga
ttgaacaagatggattgcacgcaggttctccggccgcttgggtggagaggctattcggctatgactggg
cacaacagacaatcggctgctctgatgccgccgtgttccggctgtcagcgcaggggcgcccggttcttt
ttgtcaagaccgacctgtccggtgccctgaatgaactgcaagacgaggcagcgcggctatcgtggctgg
ccacgacgggcgttccttgcgcagctgtgctcgacgttgtcactgaagcgggaagggactggctgctat
tgggcgaagtgccggggcaggatctcctgtcatctcaccttgctcctgccgagaaagtatccatcatgg
ctgatgcaatgcggcggctgcatacgcttgatccggctacctgcccattcgaccaccaagcgaaacatc
gcatcgagcgagcacgtactcggatggaagccggtcttgtcgatcaggatgatctggacgaagagcatc
aggggctcgcgccagccgaactgttcgccaggctcaaggcgagcatgcccgacggcgaggatctcgtcg
tgacccatggcgatgcctgcttgccgaatatcatggtggaaaatggccgcttttctggattcatcgact
gtggccggctgggtgtggcggaccgctatcaggacatagcgttggctacccgtgatattgctgaagagc
ttggcggcgaatgggctgaccgcttcctcgtgctttacggtatcgccgctcccgattcgcagcgcatcg
ccttctatcgccttcttgacgagttcttctgaattattaacgcttacaatttcctgatgcggtattttc
tccttacgcatctgtgcggtatttcacaccgcatcaggtggcacttttcggggaaatgtgcgcggaacc
cctatttgtttatttttctaaatacattcaaatatgtatccgctcatgagattatcaaaaaggatcttc
acctagatccttttaaattaaaaatgaagttttaaatcaatctaaagtatatatgagtaaacttggtct
gacagttaccaatgcttaatcagtgaggcacctatctcagcgatctgtctatttcgttcatccatagtt
gcctgactccccgtcgtgtagataactacgatacgggagggcttaccatctggccccagtgctgcaatg
ataccgcgagacccacgctcaccggctccagatttatcagcaataaaccagccagccggaagggccgag
cgcagaagtggtcctgcaactttatccgcctccatccagtctattaattgttgccgggaagctagagta
agtagttcgccagttaatagtttgcgcaacgttgttgccattgctacaggcatcgtggtgtcacgctcg
tcgtttggtatggcttcattcagctccggttcccaacgatcaaggcgagttacatgatcccccatgttg
tgcaaaaaagcggttagctccttcggtcctccgatcgttgtcagaagtaagttggccgcagtgttatca
ctcatggttatggcagcactgcataattctcttactgtcatgccatccgtaagatgcttttctgtgact
ggtgagtactcaaccaagtcattctgagaatagtgtatgcggcgaccgagttgctcttgcccggcgtca
atacgggataataccgcgccacatagcagaactttaaaagtgctcatcattggaaaacgttcttcgggg
cgaaaactctcaaggatcttaccgctgttgagatccagttcgatgtaacccactcgtgcacccaactga
tcttcagcatcttttactttcaccagcgtttctgggtgagcaaaaacaggaaggcaaaatgccgcaaaa
aagggaataagggcgacacggaaatgttgaatactcatactcttcctttttcaatattattgaagcatt
tatcagggttattgtctcatgaccaaaatcccttaacgtgagttttcgttccactgagcgtcagacccc
gtagaaaagatcaaaggatcttcttgagatcctttttttctgcgcgtaatctgctgcttgcaaacaaaa
aaaccaccgctaccagcggtggtttgtttgccggatcaagagctaccaactctttttccgaaggtaact
ggcttcagcagagcgcagataccaaatactgttcttctagtgtagccgtagttaggccaccacttcaag
aactctgtagcaccgcctacatacctcgctctgctaatcctgttaccagtggctgctgccagtggcgat
aagtcgtgtcttaccgggttggactcaagacgatagttaccggataaggcgcagcggtcgggctgaacg
gggggttcgtgcacacagcccagcttggagcgaacgacctacaccgaactgagatacctacagcgtgag
ctatgagaaagcgccacgcttcccgaagggagaaaggcggacaggtatccggtaagcggcagggtcgga
acaggagagcgcacgagggagcttccagggggaaacgcctggtatctttatagtcctgtcgggtttcgc
cacctctgacttgagcgtcgatttttgtgatgctcgtcaggggggcggagcctatggaaaaacgccagc
aacgcggcctttttacggttcctggccttttgctggccttttgctcacatgttctttcctgcgttatcc
cctgattctgtggataaccgtattaccgcctttgagtgagctgataccgctcgccgcagccgaacgacc
gagcgcagcgagtcagtgagcgaggaagcggaagagcgcccaatacgcaaaccgcctctccccgcgcgt
tggccgattcattaatgcagctggcacgacaggtttcccgactggaaagcgggcagtgagcgcaacgca
attaatgtgagttagctcactcattaggcaccccaggctttacactttatgcttccggctcgtatgttg
tgtggaattgtgagcggataacaatttcacacaggaaacagctatgaccatgattacgccaagctcaga
attaaccctcactaaagggactagtcctgcaggtttaaacgaattcgccctttgctttctctgaccagc
attctctcccctgggcctgtgccgctttctgtctgcagcttgtggcctgggtcacctctacggctggcc
cagatccttccctgccgcctccttcaggttccgtcttcctccactccctcttccccttgctctctgctg
tgttgctgcccaaggatgctctttccggagcacttccttctcggcgctgcaccacgtgatgtcctctga
gcggatcctccccgtgtctgggtcctctccgggcatctctcctccctcacccaaccccatgccgtcttc
actcgctgggttcccttttccttctccttctggggcctgtgccatctctcgtttcttaggatggccttc
tccgacggatgtctcccttgcgtcccgcctccccttcttgtaggcctgcatcatcaccgtttttctgga
caaccccaaagtaccccgtctccctggctttagccacctctccatcctcttgctttctttgcctggaca
ccccgttctcctgtggattcgggtcacctctcactcctttcatttgggcagctcccctaccccccttac
ctctctagtctgtgctagctcttccagccccctgtcatggcatcttccaggggtccgagagctcagcta
gtcttcttcctccaacccgggcccctatgtccacttcaggacagcatgtttgctgcctccagggatcct
gtgtccccgagctgggaccaccttatattcccagggccggttaatgtggctctggttctgggtactttt
atctgtcccctccaccccacagtggggcaagcttctgacctcttctcttcctcccacagggcctcgaga
gtttaaacccgctgatcagcctcgactgtgccttctagttgccagccatctgttgtttgcccctccccc
gtgccttccttgaccctggaaggtgccactcccactgtcctttcctaataaaatgaggaaattgcatcg
cattgtctgagtaggtgtcattctattctggggggtggggtggggcaggacagcaagggggaggattgg
gaagacaatagcaggcatgctggggatgcggtgggctctatgggtcgaccaattgcattcattttatgt
ttcaggttcagggggaggtgtgggaggttttttaaagcaagtaaaacctctacaaatgtggtatggctg
attatgatccggacgcgatcgacgtcgatcgcgtcgtacgtatgatccttatcacaccttgctcttctt
cttggggagctccacccactcgtgcaggctgcccaggggcttgcccaggctggtcagctgggcgatggc
ggtctcgtgctgctccacgaagccgccgtcctccacgtaggtcttctccaggcggtgctggatgaagtg
gtactcggggaagtccttcaccacgcccttgctcttcatcagggtgcgcatgtggcagctgtagaactt
gccgctgttcaggcggtacaccaggatcacctggcccaccagcacgccgtcgttcatgtacaccacctc
gaagctgggctgcaggccggtgatggtcttcttcatcacggggccgtcgttggggaagttgcggccctt
gtactccacgcggtacacgaacatctcctcgatcaggttgatgtcgctgcggatctccaccaggccgcc
gtcctcgtagcgcagggtgcgctcgtacacgaagccggcggggaagctctggatgaagaagtcgctgat
gtcctcggggtacttggtgaaggtgcggttgccgtactggaaggcggggctcaggatgtcgaaggcgaa
gggcaggggggcgcccttggtcacgcggatctgcaccagctggttgccgaacaggatgttgcccttgcc
gcagccctccatggtgaacacgtggttgttcaccacgccctccaggttcaccttgaagctcatgatctc
ctgcaggccggtgttcttcaggatctgcttgctcaccatggtgctagcaaccggtacccggggatcctc
tagagtcgactgatcctgcgagaatagccgggcgcgctgtgagccgaagtcgcccccgccctggccact
tccggcgcgccgagtccttaggccgccagggggcgccggcgcgcgcccagattggggacaaaggaagcc
gggccggccgcgttattaccataaaaggcaaacactggtcggaggcgtccccgcggcgcgcggcaggaa
gccaggccccaaccccctcccaaccgggcgccagccccgcctccgcccggttcaaacagcgccgggtcg
gcgcgcgcgcacgcagcggccacaccctcgggcggccagcggctcgggcaggaagtgcgcgcaagcgcc
cgggagccgcggcgaccccaccccttccggccgagcccgccttcgccccagcccaggccgcggcacccc
gggccccagaacgcacgcgcaattagcgccttgagtcccagcgcgcacgcgcaattagcgccaattccc
agcgcgcacgcagttagcgcccaaaggaccagcgcgcacgcgcaatggcgccccagcccccaccgggcc
tggcgggggctacgccgcgcccaccctgcgatccccattggcaagagcccggctcagacaaagaccccg
ccggttgcccccgccccgagagcggcacccccggagcgcgcctcccgagcgcggcctcgcgcctccgaa
ctggcgtgggggtgtcccccatctccggaggcccaggggcttctcccgcgccccccacggcggtccggt
tcccccccccatgcgccccccgctgcggcccagacggcggctctgcacgggcgaaggggccgcggccgc
atgccccggtcggctggccgggcttacctggcggcgggtgtggacgggcggcggatcggcaaaggcgag
gctctgtgctcgcggggcggacgcggtctcggcggtggtggcgcgtcgcgccgctgggttttatagggc
gccgccgcggccgctcgagccataaaaggcaactttcggaacggcgcacgctgattggccccgcgccgc
tcactcaccggcttcgccgcacagtgcagcatttttttaccccctctcccctccttttgcgaaaaaaaa
aaagagcgagagcgagattgaggaagaggaggagggagagttttggcgttggccgccttggggtgctgg
gcccgggggctgggggcgcgcgccgtggcccccgcgccccacgctgggcagtgcccggttcggccccgc
atggccaggcctgcccccggcctgcccgtctctcgggccccccacccaccgcgggacatcctaggacgc
gtgaatcgattggtcgacagatctaacttgtttattgcagcttataatggttacaaataaggcaatagc
atcacaaatttcacaaataaggcatttttttcactgcattctagttttggtttgtccaaactcatcaat
gtatcttatcatgtctggatcttacccggggagcatgtcaaggtcaaaatcgtcaagagcgtcagcagg
cagcatatcaaggtcaaagtcgtcaagggcatcggctgggagcatgtctaagtcaaaatcgtcaagggc
gtcggtcggcccgccgctttcgcactttagctgtttctccaggccacatatgattagttccaggccgaa
aaggaaggcaggttcggctccctgccggtcgaacagctcaattgcttgtttcagaagtgggggcataga
atcggtggtaggtgtctctctttcctcttttgctacttgatgctcctgttcctccaatacgcagcccag
tgtaaagtggcccacggcggacagagcgtacagtgcgttctccagggagaagccttgctgacacaggaa
cgcgagctgattttccagggtttcgtactgtttctctgttgggcgggtgccgagatgcactttagcccc
gtcgcgatgtgagaggagagcacagcggtatgacttggcgttgttccgcagaaagtcttgccatgactc
gccttccagggggcaggagtgggtatgatgcctgtccagcatctcgattggcagggcatcgagcagggc
ccgcttgttcttcacgtgccagtacagggtaggctgctcaactcccagcttttgagcgagtttccttgt
cgtcaggccttcgataccgactccattgagtaattccagagcagagtttatgactttgctcttgtccag
tctagacatggtaattcgatgatccccctggggagagaggtcggtgattcggtcaacgagggagccgac
tgccgacgtgcgctccggaggcttgcagaatgcggaacaccgcgcgggcaggaacagggcccacactac
cgccccacaccccgcctcccgcaccgccccttcccggccgctgctctcggcgcgccctgctgagcagcc
gctattggccacagcccatcgcggtcggcgcgctgccattgctccctggcgctgtccgtctgcgagggt
actagtgagacgtgcggcttccgtttgtcacgtccggcacgccgcgaaccgcaaggaaccttcccgact
taggggcggagcaggaagcgtcgccggggggcccacaagggtagcggcgaagatccgggtgacgctgcg
aacggacgtgaagaatgtgcgagacccagggtcggcgccgctgcgtttcccggaaccacgcccagagca
gccgcgtccctgcgcaaacccagggctgccttggaaaaggcgcaaccccaaccccgtggaattatcacc
tcgagaataaaatatctttattttcattacatctgtgtgttggttttttgtgtgaatcgatagtactaa
catacgctctccatcaaaacaaaacgaaacaaaacaaactagcaaaataggctgtccccagtgcaagtg
caggtgccagaacatttctctctcgagtttactccctatcagtgatagagaacgtatgaagagtttact
ccctatcagtgatagagaacgtatgcagactttactccctatcagtgatagagaacgtataaggagttt
actccctatcagtgatagagaacgtatgaccagtttactccctatcagtgatagagaacgtatctacag
tttactccctatcagtgatagagaacgtatatccagtttactccctatcagtgatagagaacgtataag
ctttgcttatgtaaaccagggcgcctataaaagagtgctgattttttgagtaaacttcaattccacaac
acttttgtcttataccaactttccgtaccacttcctaccctcgtaaagaattcggatcccgtcgagaat
tagcttcgccaccatgaagttatgggatgtcgtggctgtctgcctggtgctgctccacaccgcgtccgc
cttcccgctgcccgccggtaagaggcctcccgaggcgcccgccgaagaccgctccctcggccgccgccg
cgcgcccttcgcgctgagcagtgactcaaatatgccagaggattatcctgatcagttcgatgatgtcat
ggattttattcaagccaccattaaaagactgaaaaggtcaccagataaacaaatggcagtgcttcctag
aagagagcggaatcggcaggctgcagctgccaacccagagaattccagaggaaaaggtcggagaggcca
gaggggcaaaaaccggggttgtgtcttaactgcaatacatttaaatgtcactgacttgggtctgggcta
tgaaaccaaggaggaactgatttttaggtactgcagcggctcttgcgatgcagctgagacaacgtacga
caaaatattgaaaaacttatccagaaatagaaggctggtgagtgacaaagtagggcaggcatgttgcag
acccatcgcctttgatgatgacctgtcgtttttagatgataacctggtttaccatattctaagaaagca
ttccgctaaaaggtgtggatgtatctgaggatcaattcacgcgtggtacctctagagtcgacccccctc
gagggaattccgataatcaacctctggattacaaaatttgtgaaagattgactggtattcttaactatg
ttgctccttttacgctatgtggatacgctgctttaatgcctttgtatcatgctattgcttcccgtatgg
ctttcattttctcctccttgtataaatcctggttgctgtctctttatgaggagttgtggcccgttgtca
ggcaacgtggcgtggtgtgcactgtgtttgctgacgcaacccccactggttggggcattgccaccacct
gtcagctcctttccgggactttcgctttccccctccctattgccacggcggaactcatcgccgcctgcc
ttgcccgctgctggacaggggctcggctgttgggcactgacaattccgtggtgttgtcggggaagctga
cgtcctttccatggctgctcgcctgtgttgccacctggattctgcgcgggacgtccttctgctacgtcc
cttcggccctcaatccagcggaccttccttcccgcggcgtcgac (SEQ ID NO: 5)

1. A method to generate induced pluripotent stem cell (iPSC)-derived neuronal progenitor cells (NPCs), the method comprising:
providing a quantity of induced pluripotent stem cells (iPSCs);
culturing the iPSCs in the presence of a RHO kinase inhibitor;
generating a monolayer of cells;
further culturing the monolayer of cells in the presence of LDN and SB; and
additionally culturing in the presence of FGF, EGF and LIF to generate iPSC-derived NPCs.

2. The method of claim 1, wherein the quantity of iPSCs are provided in suspension.

3. The method of claim 1, wherein generating the monolayer comprises shaking the cultured iPSCs.

4. The method of claim 1, wherein the monolayer of cells are cultured in the presence of LDN and SB for 7-13 days.

5. (canceled)

6. The method of claim 1, wherein the iPSC-derived NPCs are aggregated as neurospheres or the iPSC-derived NPCs are engrafting iPSC-derived NPCs.

7. (canceled)

8. A method, comprising:
providing a quantity of the iPSC-derived NPCs made by the method of claim 1; and
introducing at least one vector into the iPSC-derived NPCs.

9. The method of claim 8, wherein the at least one vector is introduced by one or more of: nucleofection, transfection and electroporation.

10. The method of claim 8, wherein:
the at least one vector comprises:
an expression cassette comprising a constitutive or inducible promoter operably linked to a sequence encoding a protein or peptide; and
at least one homologous recombination sequence: or
the at least one vector comprises a piggyBac vector and a pBase vector, and the piggyBac vector comprises:
an expression cassette, comprising:
a constitutive promoter;
an inducible, bi-directional polycistronic promoter comprising a tet responsive element; and
a sequence encoding a protein or peptide;
two transposon elements, wherein the two transposon elements flank the expression cassette; and
at least one homologous recombination sequence.

11. (canceled)

12. The method of claim 10, wherein the protein or peptide comprises a neurotrophic factor.

13. The method of claim 12, wherein the neurotrophic factor comprises glial derived neurotrophic factor (GDNF).

14. The method of claim 10, wherein the at least one homologous recombination sequence comprises a sequence capable of targeting a genomic safe harbor.

15. The method of claim 14, wherein the genomic safe harbor is one of: the adeno-associated virus site 1 (AAVS1), the chemokine (C—C motif) receptor 5 (CCR5) gene, or human ortholog of mouse Rosa26 locus.

16. (canceled)

17. A quantity of cells made by the method of claim 1.

18. The quantity of cells of claim 17, wherein the cells express a genomically integrated expression cassette.

19. The quantity of cells of claim 18, wherein the genomically integrated expression cassette is at a genomic safe harbor.

20-24. (canceled)

25. The method of claim 10, wherein the constitutive promoter operably linked to the sequence encoding the protein or peptide is 3-phosphoglycerate kinase (PGK promoter).

26-27. (canceled)

28. The method of claim 10, wherein the inducible promoter operably linked to the sequence encoding the protein or peptide comprises a promoter regulated by a tetracycline-class antibiotic.

29. The method of claim 28, wherein the tetracycline-class antibiotic comprises doxycycline.

30. The method of claim 10, wherein the inducible promoter operably linked to the sequence encoding the protein or peptide is regulated by a reverse tetracycline-controlled transactivator (rtTA) or a tet-On advanced transactivator (rtTA2S-M2).

31. (canceled)

32. A quantity of cells made by the method of claim 10, wherein the iPSC-derived NPCs express a genomically integrated expression cassette.

33. The quantity of cells of claim 32, wherein the genomically integrated expression cassette is at a genomic safe harbor.
